(19) United States
(12) Patent Application Publication
(10) Pub. No.: US 2024/0368145 A1
(43) Pub. Date: Nov. 7, 2024

(54) METHODS FOR TREATING CANCER

(71) Applicant: IMMUNIC AG, Gräfelfing (DE)

(72) Inventors: Hella KOHLHOF, München (DE); Evelyn PEELEN, München (DE); Christian GEGE, Ehingen (DE); Daniel VITT, Germering (DE); Andreas MÜHLER, München (DE)

(21) Appl. No.: 18/576,533

(22) PCT Filed: Jul. 8, 2022
(86) PCT No.: PCT/EP2022/069174

§ 371 Date: Jan. 4, 2024

(30) Foreign Application Priority Data
Jul. 9, 2021 (EP) 21184795.9

(51) Int. Cl. C07D 413/14; A61K 31/422; A61K 31/427; A61K 31/506; A61K 31/5377; A61K 45/06; A61P 35/00; C07D 413/04; C07D 417/14

(52) U.S. Cl. CPC C07D 413/14; A61K 31/422; A61K 31/427; A61K 31/506; A61K 31/5377; A61K 45/06; A61P 35/00; C07D 413/04; C07D 417/14

(57) ABSTRACT

The present invention provides methods comprising one or compounds according to Formula (I)

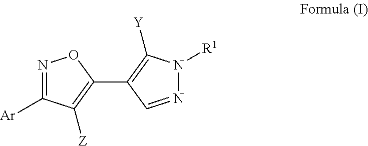

alone or in combination with one or more anticancer drugs, such as an anti-androgen drug, that are useful for treating cancer, e.g. prostate cancer, such as castration-resistant prostate cancer (CRPC) and numerous other types of cancer including lung cancer, breast cancer, liver cancer, ovarian cancer, endometrial cancer, bladder cancer, colon cancer, lymphoma and glioma.

### SUMMARY OF THE INVENTION

The present invention provides compounds according to Formula (I), alone or in combination with one or more anticancer drugs, such as an anti-androgen drug, compositions and kits comprising one or more compounds according to Formula (I) and their applications for treating cancer, e.g. prostate cancer, such as castration-resistant prostate cancer (CRPC) and numerous other types of cancer including lung cancer, breast cancer, liver cancer, ovarian cancer, endometrial cancer, bladder cancer, colon cancer, lymphoma and glioma.

### BACKGROUND OF THE INVENTION

Cancer is a leading cause of death in developed countries and despite the development of various different treatment methods such as chemotherapy, radiation therapy, vaccination and hormone deprivation therapy, there is no fully 100% effective cure to these diseases. One of the reasons current cancer treatment methods do not result in eradication of the cancerous tissue in afflicted individuals is through the development of drug resistance by the cancerous cells. Patients who exhibit drug resistance to a particular cancer drug will have tumors that no longer react to the drug and can continue growing despite continued treatment. Because drug resistance can be a common outcome during the course of administering a particular cancer therapy, it is important to continue developing new drugs and to identify new targets to treat cancer. For example, advanced prostate cancers (PCa) such as metastatic castration-resistant prostate cancers (mCRPCs) are deadly diseases. The most effective therapeutics such as enzalutamide and abiraterone give only a few months of overall survival benefit in a subgroup of the PCa patients. As such, there is currently a need in the art for new methods and compositions for treating cancer patients and patients with drug-resistant cancers. We surprisingly found that compounds according to Formula (I) potently inhibit cancer progression in various cell lines and therefore a useful for the treatment of various cancer types and malignancies, particularly RORγ-dependent cancers, such as prostate cancer.

### INTRODUCTION AND PRIOR ART

The IL-17 family of cytokines has been associated with the pathogenesis of autoimmune diseases and is generally blamed for the pathogenic symptoms of autoimmune and chronic inflammation. Overexpression of IL-17 is a hallmark of autoimmune and chronic inflammatory diseases like rheumatoid arthritis, psoriasis and psoriatic arthritis, ankylosing spondylitis, inflammatory bowel disease, multiple sclerosis, vasculitis and atherosclerosis, systemic lupus erythematosus, as well as lung disorders, asthma and chronic obstructive pulmonary diseases (Nat. Rev. Drug Discov. 2012; 11:763). The IL-17 cytokine family comprises six members, out of which IL-17A and IL-17F are the best characterized. IL-17A and IL-17F are clearly associated with inflammation, whereas the role of the other IL-17 family members is less explored (Cytokine Growth Factor Rev. 2010; 21:413). Secretion of IL-17 is mainly caused by a specific subtype of T helper cells termed T_{H}17 cells. Differentiation of naïve CD4⁺ T cells into T_{H}17 cells is induced in the presence of the cytokines IL-1β, TGFβ and IL-6, whereas IL-23 maintains T_{H}17 cell survival. Important transcription factors for the transcription and secretion of IL-17 from T_{H}17 cells are retinoic acid-related orphan receptor gamma (RORγt) and STAT3. IL-17 itself induces production of effector molecules in IL17R expressing cells like endothelial cells, epithelial cells or fibroblasts, macrophages and dendritic cells, chondrocytes and osteoblasts. Those effector molecules are pro-inflammatory cytokines (IL-6, TNFα and IL-1b), chemokines (like CXCL1, CXCL2, CXCL5, CCL2, CCL7 and CCL20), growth factors (G-CSF, GM-CSF) and nitric oxide, prostaglandin E2 and matrix-metalloproteases. Initiated by these effector molecules, neutrophil infiltration, tissue damage and chronic inflammation occurs (Nat. Rev. Drug Discov. 2012; 11:763).

Before the recognition of the importance of IL-17 in autoimmune inflammation, IFNγ derived from T_{H}1 cells was believed to be the important cytokine that drives autoimmune disorders. IFNγ transcription and secretion from T_{H}1 effector cells is regulated by the transcription factors T-bet and STAT4. As an effector cytokine of TH immunity, IFNγ is the key regulator of macrophage activation. In parallel, INFγ signaling generates other cytokines and inflammatory factors to sustain inflammation, maintain T_{H}1 responses and inhibit differentiation of regulatory T cells, T_{H}2 cells and T_{H}17 cells (Discov. Med. 2013; 16:123; J. Biol. Chem. 2017; 292:13925).

Recently, the existence of hybrid T_{H}1/T_{H}17 cells was described. These cells can be induced in vitro by IL-23 and IL-6 in concert with IL-1 and secrete IL-17 and IFNγ. It was demonstrated that these double producing cells harbor pronounced pro-inflammatory properties and are involved in the pathogenesis of inflammatory bowel disease, experimental autoimmune encephalomyelitis and type 1 diabetes.

The effectiveness of blocking IL-17 signaling alone as therapeutic treatment in autoimmune diseases has already been proven in clinical trials with e.g. monoclonal antibodies against IL-17A (sekukinumab or ixekizumab) and/or the IL-17 receptor IL-17RA (brodalumab). On the other hand, blocking the IFNγ signaling alone in autoimmune diseases with IFNγ-specific monoclonal antibody AMG811 was investigated in clinical trials for systemic and discoid lupus erythematosus without significant clinical benefit so far.

Compounds which target and suppress both IL-17 and IFNγ are therefore predestined for the treatment of autoimmune disorders.

Of note, a second isoform of RORγ exist beside RORγt, which is an elongated form with a longer amino terminal domain and this isoform is expressed in many tissues, including thymus, lung, liver, kidney, muscle and brown fat. Since both isoforms have the same ligand binding domain (LBD), a small molecule compound always modulates both isoforms in a similar manner.

The androgen receptor (AR) is overexpressed and hyperactivated in human castration-resistant prostate cancer (CRPC). It was shown by J. Wang et al. (Nat. Med. 2016; 22:488 or WO2016/145298) that RORγt is overexpressed and amplified in metastatic CRPC tumors and that RORγt drives AR expression in the tumors. RORγt recruits coactivators SRC-1 and -3 to an AR-ROR response element to stimulate AR gene transcription. RORγ antagonists suppress the expression of AR and its variant AR-V7 in prostate cancer cell lines and tumors. RORγt antagonists also markedly diminish genome-wide AR binding, H3K27ac abundance and expression of the AR gene network. Lastly J. Wang et al. showed, that RORγt antagonists suppress tumor growth in multiple AR-expressing but not AR-negative xenograft PCa models and effectively sensitized CRPC tumors to enzalutamide, without overting toxicity in mice.

WO2012/101261, WO2012/101263 and WO2019/048541 describe compounds of Formula (I) of the present invention which are limited towards the treatment of autoimmune and chronic inflammatory diseases. We surprisingly found that compounds according Formula (I) potently inhibit cancer progression in various cell lines and therefore are useful for the treatment of various cancer types and malignancies, particularly RORγ-dependent cancers, such as prostate cancer.

### DETAILED DESCRIPTION OF THE INVENTION

It is an object of the present invention to provide effective agents, compositions, methods and kits comprising one or more compounds according Formula (I) alone or in combination with one or more anticancer drugs, such as an anti-androgen drug, that are useful for treating cancer, e.g. prostate cancer, such as castration-resistant prostate cancer (CRPC) and numerous other types of cancer including lung cancer, breast cancer, liver cancer, ovarian cancer, endometrial cancer, bladder cancer, colon cancer, lymphoma and glioma.

The present invention relates to a compound according to Formula (I):

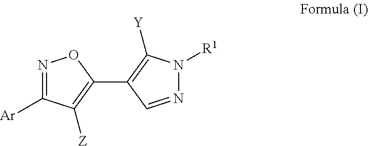

and a pharmaceutically acceptable salt, a solvate, a solvate of a salt, a hydrate or a polymorph thereof for use in a method of treating cancer in a subject in need thereof, wherein
Ar is selected from the group consisting of phenyl and heteroaryl, each of which is optionally substituted by one or more independently selected substituents R^{Ar};
R^{Ar} is selected from the group consisting of halogen, —OH, —CN, alkoxy, haloalkoxy, alkyl, haloalkyl, mono- or dialkylamino-alkyl, mono- or di-alkylamino-alkoxy, —COOR′, —CONHR′, —CO—R′, —SO₂NHR′, —NH—CO—R′, —NO₂, —NH—SO₂—R′, —SO₂—R′, benzyloxy, —CO-heterocyclyl, —CO-cycloalkyl, —CONH-cycloalkyl, —CONH-heterocyclyl, —O-alkyl-heterocyclyl, —O-alkyl-cycloalkyl, (2-oxa-6-azaspiro[3.3]hept-6-yl)-C₁₋₄-alkoxy, amino, arylalkyl, cycloalkyl, heterocyclyl, phenyl and heteroaryl, wherein each of said alkoxy, arylalkyl, alkyl, cycloalkyl, heterocyclyl, phenyl and heteroaryl groups is optionally substituted by one or more substituents independently selected from alkyl, haloalkyl, halogen and OH;
R′ is independently selected from the group consisting of H, OH, alkyl and haloalkyl;
Z is selected from the group consisting of H, halogen, —CO—R^{Z}, —CH₂—O—R^{Z}, —CO—CH₂—R^{Z}, —CO—CH₂—O—R^{Z}, —COOR^{Z}, —NHCO—R^{Z}, —CO—NHR^{Z}, —N(R^{Z})₂, —CN, —NHCOOR^{Z}, —SO₂—R^{Z}, —SO₂NHR^{Z}, -alkyl-O—R^{Z}, -alkyl-O-alkyl-O—R^{Z}, amino, alkyl, phenyl, heteroaryl, heterocyclyl and cycloalkyl, wherein each of said alkyl, phenyl, heteroaryl, heterocyclyl and cycloalkyl groups is optionally substituted by one or more substituents independently selected from the group consisting of halogen, alkyl, alkoxy, haloalkyl, —COO-alkyl, OH and cycloalkyl;
R^{Z} is selected from the group consisting of H, halogen, —OH, alkyl, haloalkyl, cycloalkyl, heterocyclyl, phenyl and heteroaryl;
Y is selected from H, halogen, haloalkyl, alkyl or an alkylester;
R′ is selected from aryl, heteroaryl, heterocyclyl or alkyl, which can be substituted by one or more substituents R″; wherein
R″ is independently selected from H, —CO₂R′″, —CONHR′″, —CR′″O, —SO₂N(R′″)₂, —SO₂NHR′″, —NR′″-CO-haloalkyl, —NO₂, —NR′″-SO₂-haloalkyl, —NR′″-SO₂-alkyl, —SO₂-alkyl, —NR′″—CO-alkyl, —CN, alkyl, cycloalkyl, aminoalkyl, alkylamino, alkoxy, —OH, —SH, alkylthio, hydroxyalkyl, hydroxyalkylamino, halogen, haloalkyl, haloalkoxy, amino, heterocyclyl, aryl, haloaryl, haloarylalkyl, arylalkyl or heteroaryl;
R′″ independently represents H, haloalkyl, hydroxyalkyl, amino, alkoxy, —N═C(R″)₂, —NR″—CO—R″, —CR″O, —CO₂R″, alkyl, cycloalkyl, aryl, haloaryl, haloarylalkyl, heteroaryl, heterocyclyl, arylalkyl or aminoalkyl, which are optionally substituted by one or more substituents R″;
wherein the longest chain allowed in R¹ are three coupled substituents R″ and/or R′″
or, alternatively R′ is a group of the structure

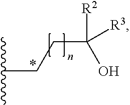

wherein
n is 0 or 1;
R² is H, deuterium or methyl;
R³ is methyl, trifluoromethyl, ethyl, or taken with R² together forms a cyclopropyl group; or
n is 1, R² is H, deuterium or methyl and R³ forms a methylene bridge to the carbon atom marked *.

One embodiment of the present invention relates to the compound according to Formula (I) in which Ar is phenyl, which is optionally substituted by one or more independently selected from fluoro, chloro, alkoxy, fluoroalkoxy, —CO-heterocyclyl and —O-alkyl-heterocyclyl.
Z is selected from the group consisting of —CO—R^{Z}, —CH₂—O—R^{Z} and heteroaryl, wherein said heteroaryl is optionally substituted by one or more substituents independently selected from the group consisting of fluoro, chloro, alkyl, alkoxy, fluoroalkyl and OH;
R^{Z} is selected from the group consisting of alkyl, fluoroalkyl, cycloalkyl and heterocyclyl;
Y is selected from alkyl or fluoroalkyl;
R¹ is selected from aryl, which can be substituted by one or more substituents selected from chloro, fluoro, —CN, alkyl, fluoroalkyl, alkoxy or fluoroalkoxy;
or a group of the structure

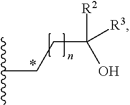

wherein
n is 0 or 1;
R² is H, deuterium or methyl;
R³ is methyl, trifluoromethyl, ethyl, or taken with R² together forms a cyclopropyl group; or
n is 1, R² is H, deuterium or methyl and R³ forms a methylene bridge to the carbon atom marked *, or a pharmaceutically acceptable salt, a solvate, a solvate of a salt, a hydrate or a polymorph thereof.

One embodiment of the present invention relates to the compound according to Formula (I) in which Ar is selected from

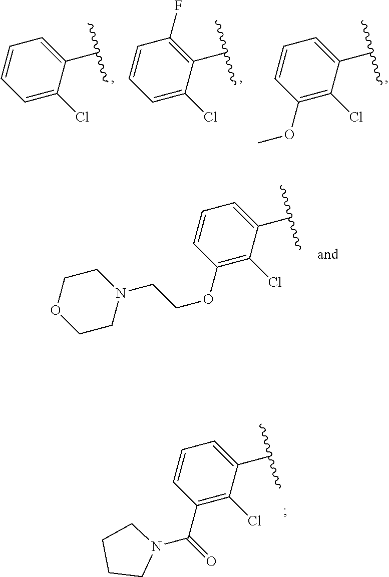

Z is selected from

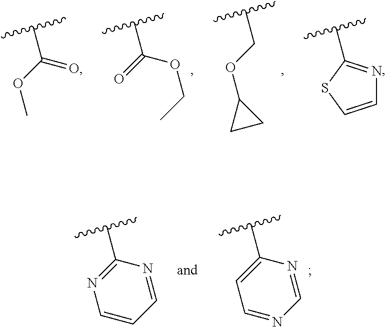

Y is CF₃;
R¹ is selected from

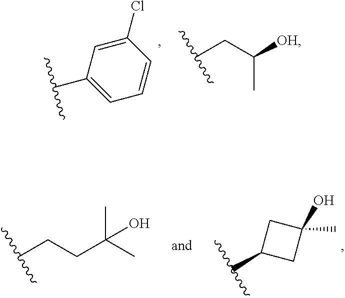

or a pharmaceutically acceptable salt, a solvate, a solvate of a salt, a hydrate or a polymorph thereof.

One embodiment of the present invention relates to the compound according to Formula (I), wherein said compound is selected from

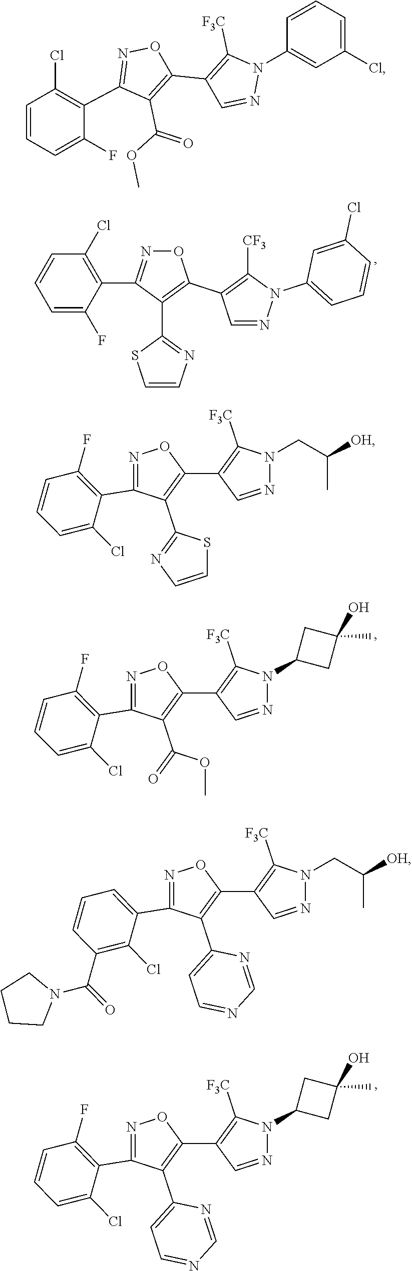 [SMILES: COC(=O)c1c(-c2c(F)cccc2Cl)noc1-c1cnn(-c2cccc(Cl)c2)c1C(F)(F)F ; Fc1cccc(Cl)c1-c1noc(-c2cnn(-c3cccc(Cl)c3)c2C(F)(F)F)c1-c1nccs1 ; C[C@H](O)Cn1ncc(-c2onc(-c3c(F)cccc3Cl)c2-c2nccs2)c1C(F)(F)F ; COC(=O)c1c(-c2c(F)cccc2Cl)noc1-c1cnn([C@H]2C[C@@](C)(O)C2)c1C(F)(F)F ; C[C@H](O)Cn1ncc(-c2onc(-c3cccc(C(=O)N4CCCC4)c3Cl)c2-c2ccncn2)c1C(F)(F)F ; C[C@]1(O)C[C@@H](n2ncc(-c3onc(-c4c(F)cccc4Cl)c3-c3ccncn3)c2C(F)(F)F)C1]

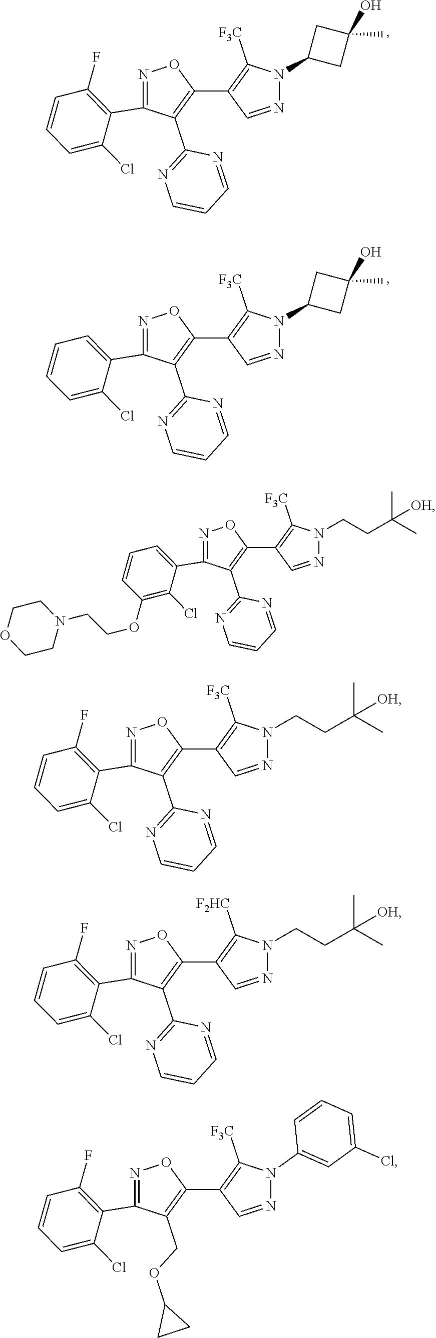

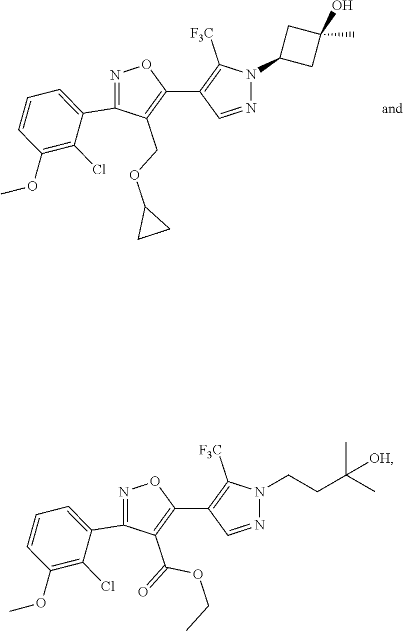

or a pharmaceutically acceptable salt, a solvate, a solvate of a salt, a hydrate or a polymorph thereof. One embodiment of the present invention relates to the compound according to Formula (I) which is

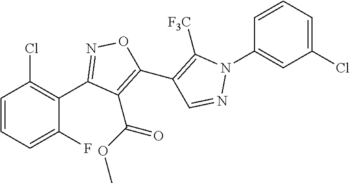 [SMILES: COC(=O)c1c(-c2c(F)cccc2Cl)noc1-c1cnn(-c2cccc(Cl)c2)c1C(F)(F)F]

or a pharmaceutically acceptable salt, a solvate, a solvate of a salt, a hydrate or a polymorph thereof.

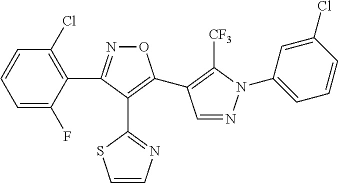 [SMILES: Fc1cccc(Cl)c1-c1noc(-c2cnn(-c3cccc(Cl)c3)c2C(F)(F)F)c1-c1nccs1]

One embodiment of the present invention relates to the compound according to Formula (I) which is

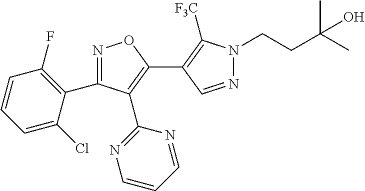 [SMILES: CC(C)(O)CCn1ncc(-c2onc(-c3c(F)cccc3Cl)c2-c2ncccn2)c1C(F)(F)F]

or a pharmaceutically acceptable salt, a solvate, a solvate of a salt, a hydrate or a polymorph thereof.

In one embodiment of the present invention the cancer is selected from the group consisting of prostate cancer, breast cancer, lung cancer, ovarian cancer, bladder cancer, endometrial cancer, liver cancer, glioblastoma, B-cell lymphoma and colon cancer.

In one embodiment of the present invention the cancer is resistant to a chemotherapeutic agent selected from tamoxifen, a taxane, an anthracenedione or combinations thereof. In one embodiment the taxane is selected from the group consisting of paclitaxel, docetaxel, cabazitaxel, hongdoushan A, hongdoushan B, hongdoushan C, baccatin I, baccatin II, 10-deacetylbaccatin or combinations thereof.

In one embodiment of the present invention the cancer is a RORγ-dependent cancer.

In one embodiment of the present invention the cancer is prostate cancer.

In one embodiment of the present invention the prostate cancer is a castration-resistant prostate cancer.

In one embodiment of the present invention the cancer is a non-small-cell lung cancer (NSCLC), K-Ras mutant lung cancer, BRAF mutant lung cancer, EGFR mutant lung cancer, tyrosine kinase inhibitor-resistant lung cancer or small cell lung cancer (SCLC).

In one embodiment of the present invention the breast cancer is a triple-negative breast cancer (TNBC), tamoxifen-resistant breast cancer, radiation-resistant breast cancer, HER2-positive breast cancer or ER-positive breast cancer.

In one embodiment of the present invention the subject is a human in need of cancer treatment.

In one embodiment of the present invention said compound according to Formula (I) is provided as a suitable pharmaceutical composition, such as a tablet, capsule, granule, powder, sachet, reconstitutable powder, dry powder inhaler and/or chewable.

The present invention further relates to a method for treating cancer in a subject in need thereof, the method comprising administering to the subject an effective amount of a compound according to Formula (I) or a pharmaceutically acceptable salt, a solvate, a solvate of a salt, a hydrate or a polymorph thereof.

In one embodiment of the present invention said compound according to Formula (I) or a pharmaceutically acceptable salt, a solvate, a solvate of a salt, a hydrate or a polymorph thereof is administered to said subject in an effective dosage regimen, for example one to three times a day with a cumulative daily dose of 150 to 2000 mg, preferably 150 to 900 mg, more preferably preferably 300 to 900 mg.

In one embodiment of the present invention said method further comprises administering to the subject an effective amount of an anticancer drug. In one embodiment, the anticancer drug is a chemotherapeutic agent selected from a taxane, an anthracenedione or combinations thereof. In one embodiment the anticancer drug is a taxane, wherein the taxane is selected from the group consisting of paclitaxel, docetaxel, cabazitaxel, hongdoushan A, hongdoushan B, hongdoushan C, baccatin I, baccatin II, 10-deacetylbaccatin or combinations thereof. In one embodiment, the anticancer drug is an endocrine therapy selected from the group consisting of tamoxifen, raloxifene, an aromatase inhibitor, megestrol acetate, lasofoxifene, bazedoxifene, bazedoxifene/conjugated estrogens or combinations thereof.

In one embodiment of the present invention the compound according to Formula (I) enhances the therapeutic effect of the anticancer drug.

In one embodiment of the present invention the compound according to Formula (I) reverses or reduces cancer cell resistance to the anticancer drug and/or sensitizes cancer cells to the anticancer drug.

The particular embodiments of the present invention are described in the following items:
1. A compound of Formula (I):

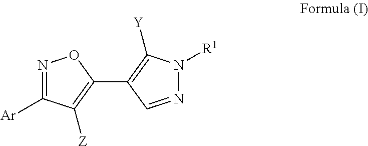

or a pharmaceutically acceptable salt, a solvate, a solvate of a salt, a hydrate or a polymorph thereof for use in a method of treating a cancer in a subject in need thereof, wherein
Ar is selected from the group consisting of phenyl and heteroaryl, each of which is optionally substituted by one or more independently selected substituents R^{Ar};
R is selected from the group consisting of halogen, —OH, —CN, alkoxy, haloalkoxy, alkyl, haloalkyl, mono- or dialkylamino-alkyl, mono- or di-alkylamino-alkoxy, —COOR′, —CONHR′, —CO—R′, —SO₂NHR′, —NH—CO—R′, —NO₂, —NH—SO₂—R′, —SO₂—R′, benzyloxy, —CO-heterocyclyl, —CO-cycloalkyl, —CONH-cycloalkyl, —CONH-heterocyclyl, —O-alkyl-heterocyclyl, —O-alkyl-cycloalkyl, (2-oxa-6-azaspiro[3.3]hept-6-yl)-C₁₋₄-alkoxy, amino, arylalkyl, cycloalkyl, heterocyclyl, phenyl and heteroaryl, wherein each of said alkoxy, arylalkyl, alkyl, cycloalkyl, heterocyclyl, phenyl and heteroaryl groups is optionally substituted by one or more substituents independently selected from alkyl, haloalkyl, halogen and OH;
R′ is independently selected from the group consisting of H, OH, alkyl and haloalkyl;
Z is selected from the group consisting of H, halogen, —CO—R^{Z}, —CH₂—O—R^{Z}, —CO—CH₂—R^{Z}, —CO—CH₂—O—R^{Z}, —COOR^{Z}, —NHCO—R^{Z}, —CO—NHR^{Z}, —N(R^{Z})₂, —CN, —NHCOOR^{Z}, —SO₂—R^{Z}, —SO₂NHR^{Z}, -alkyl-O—R^{Z}, -alkyl-O-alkyl-O—R^{Z}, amino, alkyl, phenyl, heteroaryl, heterocyclyl and cycloalkyl, wherein each of said alkyl, phenyl, heteroaryl, heterocyclyl and cycloalkyl groups is optionally substituted by one or more substituents independently selected from the group consisting of halogen, alkyl, alkoxy, haloalkyl, —COO-alkyl, OH and cycloalkyl;
R^{Z} is selected from the group consisting of H, halogen, —OH, alkyl, haloalkyl, cycloalkyl, heterocyclyl, phenyl and heteroaryl;
Y is selected from H, halogen, haloalkyl, alkyl or an alkylester;
R¹ is selected from aryl, heteroaryl, heterocyclyl or alkyl, which can be substituted by one or more substituents R″; wherein
R″ is independently selected from H, —CO₂R′″, —CONHR′″, —CR′″O, —SO₂N(R′″)₂, —SO₂NHR′″, —NR′″—CO-haloalkyl, —NO₂, —NR′″—SO₂-haloalkyl, —NR′″—SO₂-alkyl, —SO₂-alkyl, —NR′″—CO-alkyl, —CN, alkyl, cycloalkyl, aminoalkyl, alkylamino, alkoxy, —OH, —SH, alkylthio, hydroxyalkyl, hydroxyalkylamino, halogen, haloalkyl, haloalkoxy, amino, heterocyclyl, aryl, haloaryl, haloarylalkyl, arylalkyl or heteroaryl;
R′″ independently represents H, haloalkyl, hydroxyalkyl, amino, alkoxy, —N═C(R″)₂, —NR″—CO—R″, —CR″O, —CO₂R″, alkyl, cycloalkyl, aryl, haloaryl, haloarylalkyl, heteroaryl, heterocyclyl, arylalkyl or aminoalkyl, which are optionally substituted by one or more substituents R″;
wherein the longest chain allowed in R¹ are three coupled substituents R″ and/or R′″ or, alternatively R′ is a group of the structure

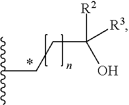

wherein
n is 0 or 1;
R² is H, deuterium or methyl;
R³ is methyl, trifluoromethyl, ethyl, or taken with R² together forms a cyclopropyl group; or
n is 1, R² is H, deuterium or methyl and R³ forms a methylene bridge to the carbon atom marked *.
2. The compound of Formula (I) or a pharmaceutically acceptable salt, a solvate, a solvate of a salt, a hydrate or a polymorph thereof for use in a method of treating a cancer in a subject in need thereof according to item 1, wherein
Ar is phenyl, which is optionally substituted by one or more independently selected from fluoro, chloro, alkoxy, fluoroalkoxy, —CO-heterocyclyl and —O-alkyl-heterocyclyl.
Z is selected from the group consisting of —CO—R^{Z}, —CH₂—O—R^{Z} and heteroaryl, wherein said heteroaryl is optionally substituted by one or more substituents independently selected from the group consisting of fluoro, chloro, alkyl, alkoxy, fluoroalkyl and OH;
R^{Z} is selected from the group consisting of alkyl, fluoroalkyl, cycloalkyl and heterocyclyl;
Y is selected from alkyl or fluoroalkyl;
R′ is selected from aryl, which can be substituted by one or more substituents selected from chloro, fluoro, —CN, alkyl, fluoroalkyl, alkoxy or fluoroalkoxy;
or a group of the structure

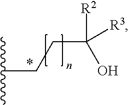

wherein
n is 0 or 1;
R² is H, deuterium or methyl;
R³ is methyl, trifluoromethyl, ethyl, or taken with R² together forms a cyclopropyl group; or
n is 1, R² is H, deuterium or methyl and R³ forms a methylene bridge to the carbon atom marked *.
3. The compound of Formula (I) or a pharmaceutically acceptable salt, a solvate, a solvate of a salt, a hydrate or a polymorph thereof for use in a method of treating a cancer in a subject in need thereof according to item 1 or 2, wherein
Ar is selected from

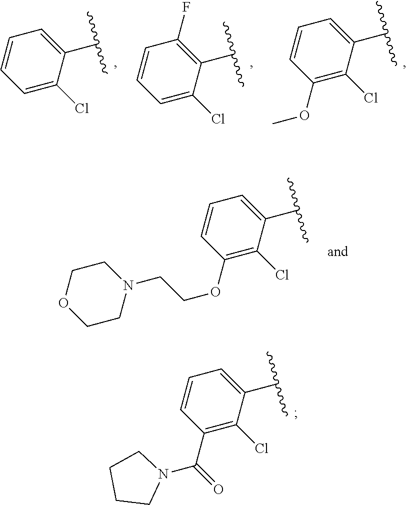

Z is selected from

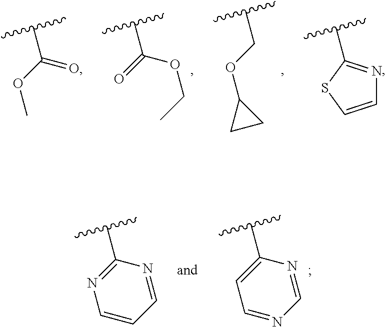

Y is CF₃;
R′ is selected from

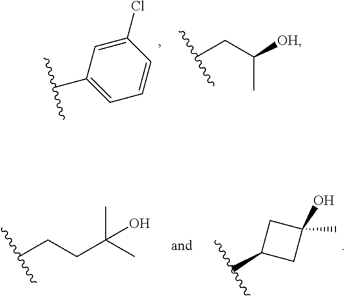

4. The compound of Formula (I) or a pharmaceutically acceptable salt, a solvate, a solvate of a salt, a hydrate or a polymorph thereof for use in a method of treating a cancer in a subject in need thereof according to any of items 1 to 3, wherein the compound according to Formula (I) is selected from

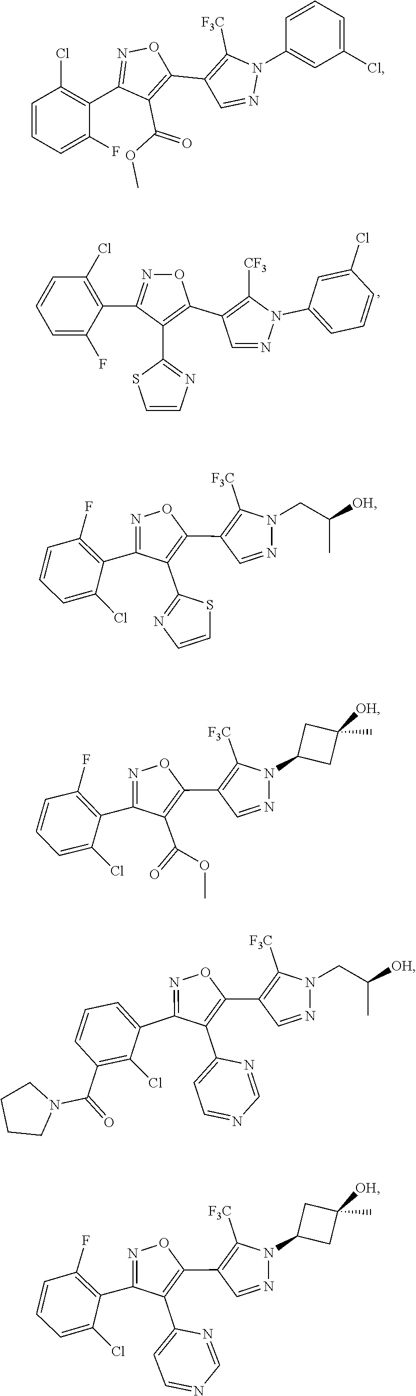 [SMILES: COC(=O)c1c(-c2c(F)cccc2Cl)noc1-c1cnn(-c2cccc(Cl)c2)c1C(F)(F)F ; Fc1cccc(Cl)c1-c1noc(-c2cnn(-c3cccc(Cl)c3)c2C(F)(F)F)c1-c1nccs1 ; C[C@H](O)Cn1ncc(-c2onc(-c3c(F)cccc3Cl)c2-c2nccs2)c1C(F)(F)F ; COC(=O)c1c(-c2c(F)cccc2Cl)noc1-c1cnn([C@H]2C[C@@](C)(O)C2)c1C(F)(F)F ; C[C@H](O)Cn1ncc(-c2onc(-c3cccc(C(=O)N4CCCC4)c3Cl)c2-c2ccncn2)c1C(F)(F)F ; C[C@]1(O)C[C@@H](n2ncc(-c3onc(-c4c(F)cccc4Cl)c3-c3ccncn3)c2C(F)(F)F)C1]

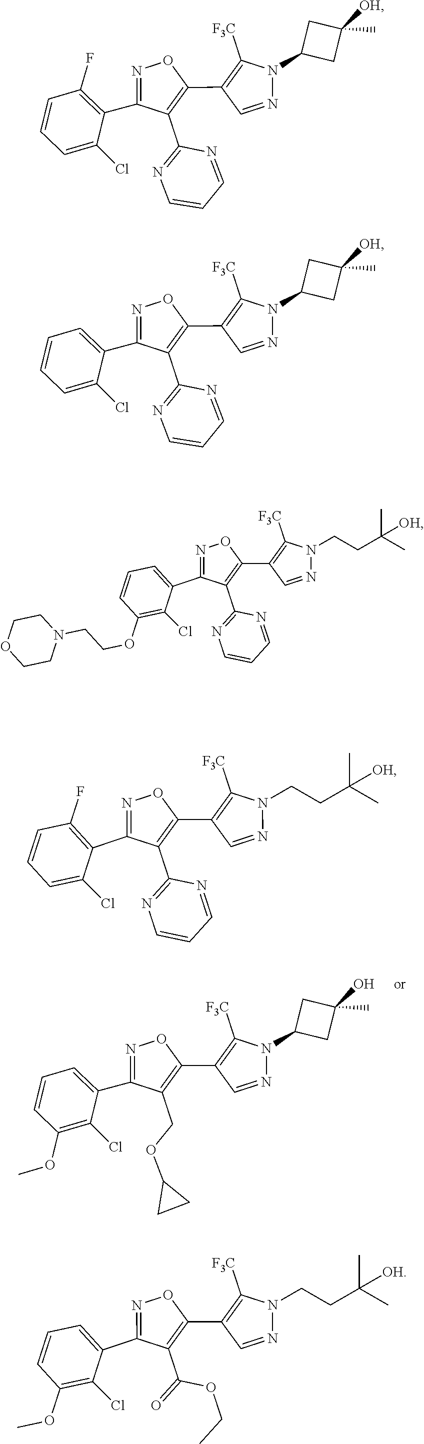

5. The compound of Formula (I) or a pharmaceutically acceptable salt, a solvate, a solvate of a salt, a hydrate or a polymorph thereof for use in a method of treating a cancer in a subject in need thereof according to any one of items 1 to 4, wherein the cancer is selected from the group consisting of prostate cancer, breast cancer, lung cancer, ovarian cancer, bladder cancer, endometrial cancer, liver cancer, glioblastoma, B-cell lymphoma and colon cancer.
6. The compound of Formula (I) or a pharmaceutically acceptable salt, a solvate, a solvate of a salt, a hydrate or a polymorph thereof for use in a method of treating a cancer in a subject in need thereof according to any one of items 1 to 5, wherein the cancer is resistant to a chemotherapeutic agent selected from tamoxifen, a taxane, an anthracenedione or combinations thereof.

In a particular embodiment of item 6, the taxane according to item 6 is selected from the group consisting of paclitaxel, docetaxel, cabazitaxel, hongdoushan A, hongdoushan B, hongdoushan C, baccatin I, baccatin II, 10-deacetylbaccatin or combinations thereof.
7. The compound of Formula (I) or a pharmaceutically acceptable salt, a solvate, a solvate of a salt, a hydrate or a polymorph thereof for use in a method of treating a cancer in a subject in need thereof according to item 5 or 6, wherein the prostate cancer is a castration-resistant prostate cancer.

In a particular embodiment of item 7, the cancer according item 5 is a non-small-cell lung cancer (NSCLC), K-Ras mutant lung cancer, BRAF mutant lung cancer, EGFR mutant lung cancer, tyrosine kinase inhibitor-resistant lung cancer or small cell lung cancer (SCLC).

In an additional particular embodiment of item 7, the breast cancer according item 5 is a triple-negative breast cancer (TNBC), tamoxifen-resistant breast cancer, radiation-resistant breast cancer, HER2-positive breast cancer or ER-positive breast cancer.
8. The compound of Formula (I) or a pharmaceutically acceptable salt, a solvate, a solvate of a salt, a hydrate or a polymorph thereof for use in a method of treating a cancer in a subject in need thereof according to any one of items 1 to 7, wherein the subject is a human in need of cancer treatment.
9. The compound of Formula (I) or a pharmaceutically acceptable salt, a solvate, a solvate of a salt, a hydrate or a polymorph thereof for use in a method of treating a cancer in a subject in need thereof according to any one of items 1 to 8, wherein said compound of Formula (I) or a pharmaceutically acceptable salt, a solvate, a solvate of a salt, a hydrate or a polymorph thereof is provided as a suitable pharmaceutical composition, such as a tablet, capsule, granule, powder, sachet, reconstitutable powder, dry powder inhaler and/or chewable.
10. The compound of Formula (I) or a pharmaceutically acceptable salt, a solvate, a solvate of a salt, a hydrate or a polymorph thereof for use in a method of treating a cancer in a subject in need thereof according to any one of items 1 to 9, wherein said compound of Formula (I) or a pharmaceutically acceptable salt, a solvate, a solvate of a salt, a hydrate or a polymorph thereof is administered to said subject in an effective dosage regimen, for example one to three times a day with a cumulative daily dose of 150 to 900 mg.
11. The compound of Formula (I) or a pharmaceutically acceptable salt, a solvate, a solvate of a salt, a hydrate or a polymorph thereof for use in a method of treating a cancer in a subject in need thereof according to any one of items 1 to 10, wherein said method further comprises administering to the subject an effective amount of an anticancer drug.

In a particular embodiment of item 11, the anticancer drug is a chemotherapeutic agent selected from a taxane, an anthracenedione or combinations thereof.

In an additional particular embodiment of item 11, the anticancer drug is a taxane, wherein the taxane is selected from the group consisting of paclitaxel, docetaxel, cabazitaxel, hongdoushan A, hongdoushan B, hongdoushan C, baccatin I, baccatin II, 10-deacetylbaccatin or combinations thereof.

In an additional particular embodiment of item 11, the anticancer drug is an endocrine therapy selected from the group consisting of tamoxifen, raloxifene, an aromatase inhibitor, megestrol acetate, lasofoxifene, bazedoxifene, bazedoxifene/conjugated estrogens or combinations thereof.
12. The compound of Formula (I) or a pharmaceutically acceptable salt, a solvate, a solvate of a salt, a hydrate or a polymorph thereof for use in a method of treating a cancer in a subject in need thereof according to item 11, wherein said compound of Formula (I) or a pharmaceutically acceptable salt, a solvate, a solvate of a salt, a hydrate or a polymorph thereof enhances the therapeutic effect of the anticancer drug.
13. The compound of Formula (I) or a pharmaceutically acceptable salt, a solvate, a solvate of a salt, a hydrate or a polymorph thereof for use in a method of treating a cancer in a subject in need thereof according to item 12, wherein said compound of Formula (I) or a pharmaceutically acceptable salt, a solvate, a solvate of a salt, a hydrate or a polymorph thereof reverses or reduces cancer cell resistance to the anticancer drug and/or sensitizes cancer cells to the anticancer drug.
14. A method for treating a cancer in a subject in need thereof, the method comprising administering to the subject an effective amount of a compound according to Formula (I):

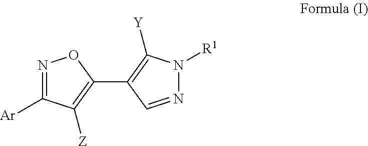

or a pharmaceutically acceptable salt, a solvate, a solvate of a salt, a hydrate or a polymorph thereof, wherein
Ar is selected from the group consisting of phenyl and heteroaryl, each of which is optionally substituted by one or more independently selected substituents R^{Ar};
R^{Ar} is selected from the group consisting of halogen, —OH, —CN, alkoxy, haloalkoxy, alkyl, haloalkyl, mono- or dialkylamino-alkyl, mono- or di-alkylamino-alkoxy, —COOR′, —CONHR′, —CO—R′, —SO₂NHR′, —NH—CO—R′, —NO₂, —NH—SO₂—R′, —SO₂—R′, benzyloxy, —CO-heterocyclyl, —CO-cycloalkyl, —CONH-cycloalkyl, —CONH-heterocyclyl, —O-alkyl-heterocyclyl, —O-alkyl-cycloalkyl, (2-oxa-6-azaspiro[3.3]hept-6-yl)-C₁₋₄-alkoxy, amino, arylalkyl, cycloalkyl, heterocyclyl, phenyl and heteroaryl, wherein each of said alkoxy, arylalkyl, alkyl, cycloalkyl, heterocyclyl, phenyl and heteroaryl groups is optionally substituted by one or more substituents independently selected from alkyl, haloalkyl, halogen and OH;
R′ is independently selected from the group consisting of H, OH, alkyl and haloalkyl;
Z is selected from the group consisting of H, halogen, —CO—R^{Z}, —CH₂—O—R^{Z}, —CO—CH₂—R^{Z}, —CO—CH₂—O—R^{Z}, —COOR^{Z}, —NHCO—R^{Z}, —CO—NHR^{Z}, —N(R^{Z})₂, —CN, —NHCOOR^{Z}, —SO₂—R^{Z}, —SO₂NHR^{Z}, -alkyl-O—R^{Z}, -alkyl-O-alkyl-O—R^{Z}, amino, alkyl, phenyl, heteroaryl, heterocyclyl and cycloalkyl, wherein each of said alkyl, phenyl, heteroaryl, heterocyclyl and cycloalkyl groups is optionally substituted by one or more substituents independently selected from the group consisting of halogen, alkyl, alkoxy, haloalkyl, —COO-alkyl, OH and cycloalkyl;
R^{Z} is selected from the group consisting of H, halogen, —OH, alkyl, haloalkyl, cycloalkyl, heterocyclyl, phenyl and heteroaryl;
Y is selected from H, halogen, haloalkyl, alkyl or an alkylester;
R¹ is selected from aryl, heteroaryl, heterocyclyl or alkyl, which can be substituted by one or more substituents R″; wherein
R″ is independently selected from H, —CO₂R′″, —CONHR′″, —CR′″O, —SO₂N(R′″)₂, —SO₂NHR′″, —NR′″—CO-haloalkyl, —NO₂, —NR′″—SO₂-haloalkyl, —NR′″—SO₂-alkyl, —SO₂-alkyl, —NR′″—CO-alkyl, —CN, alkyl, cycloalkyl, aminoalkyl, alkylamino, alkoxy, —OH, —SH, alkylthio, hydroxyalkyl, hydroxyalkylamino, halogen, haloalkyl, haloalkoxy, amino, heterocyclyl, aryl, haloaryl, haloarylalkyl, arylalkyl or heteroaryl;
R′″ independently represents H, haloalkyl, hydroxyalkyl, amino, alkoxy, —N═C(R″)₂, —NR″—CO—R″, —CR″O, —CO₂R″, alkyl, cycloalkyl, aryl, haloaryl, haloarylalkyl, heteroaryl, heterocyclyl, arylalkyl or aminoalkyl, which are optionally substituted by one or more substituents R′;
wherein the longest chain allowed in R¹ are three coupled substituents R″ and/or R′″
or, alternatively R′ is a group of the structure

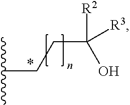

wherein
n is 0 or 1;
R² is H, deuterium or methyl;
R³ is methyl, trifluoromethyl, ethyl, or taken with R² together forms a cyclopropyl group;
or
n is 1, R² is H, deuterium or methyl and R³ forms a methylene bridge to the carbon atom marked *.
15. The method according to item 14, wherein
Ar is phenyl, which is optionally substituted by one or more independently selected from fluoro, chloro, alkoxy, fluoroalkoxy, —CO-heterocyclyl and —O-alkyl-heterocyclyl.
Z is selected from the group consisting of —CO—R^{Z}, —CH₂—O—R^{Z} and heteroaryl, wherein said heteroaryl is optionally substituted by one or more substituents independently selected from the group consisting of fluoro, chloro, alkyl, alkoxy, fluoroalkyl and OH;
R^{Z} is selected from the group consisting of alkyl, fluoroalkyl, cycloalkyl and heterocyclyl;
Y is selected from alkyl or fluoroalkyl;
R′ is selected from aryl, which can be substituted by one or more substituents selected from chloro, fluoro, —CN, alkyl, fluoroalkyl, alkoxy or fluoroalkoxy;
or a group of the structure

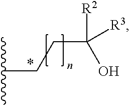

wherein
n is 0 or 1;
R² is H, deuterium or methyl;
R³ is methyl, trifluoromethyl, ethyl, or taken with R² together forms a cyclopropyl group; or
n is 1, R² is H, deuterium or methyl and R³ forms a methylene bridge to the carbon atom marked *.
16. The method according to item 14 or 15, wherein
Ar is selected from

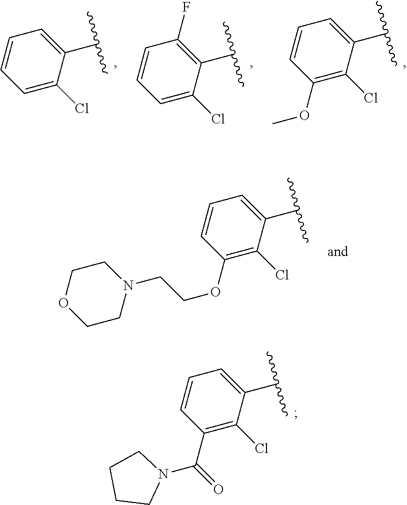

Z is selected from

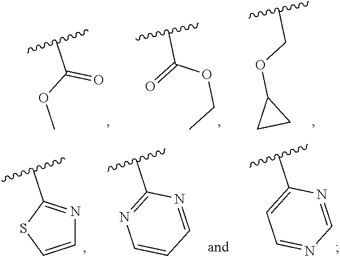

Y is CF₃;
R′ is selected from

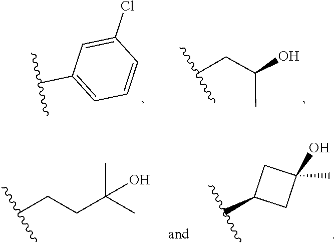

17. The method according to any of items 14 to 16, wherein the compound according to Formula (I) is selected from

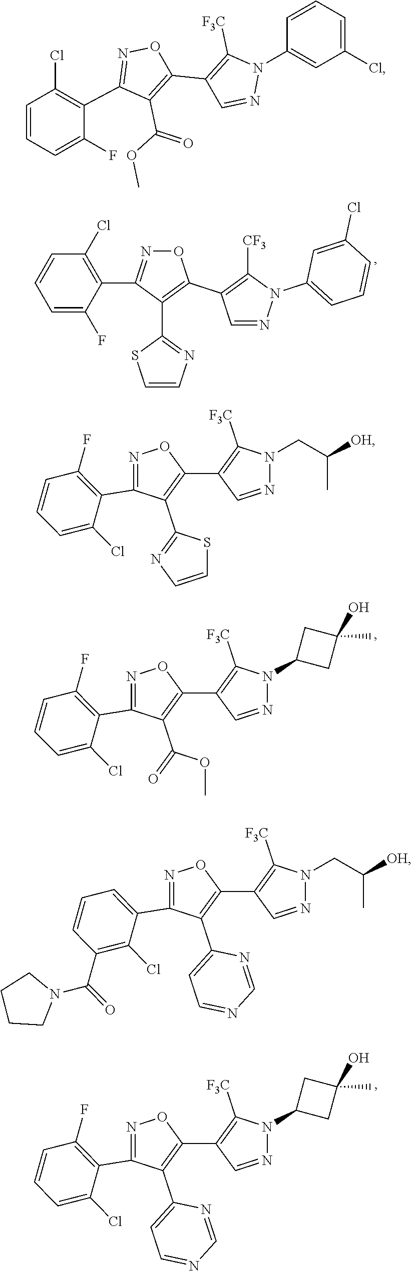 [SMILES: COC(=O)c1c(-c2c(F)cccc2Cl)noc1-c1cnn(-c2cccc(Cl)c2)c1C(F)(F)F ; Fc1cccc(Cl)c1-c1noc(-c2cnn(-c3cccc(Cl)c3)c2C(F)(F)F)c1-c1nccs1 ; C[C@H](O)Cn1ncc(-c2onc(-c3c(F)cccc3Cl)c2-c2nccs2)c1C(F)(F)F ; COC(=O)c1c(-c2c(F)cccc2Cl)noc1-c1cnn([C@H]2C[C@@](C)(O)C2)c1C(F)(F)F ; C[C@H](O)Cn1ncc(-c2onc(-c3cccc(C(=O)N4CCCC4)c3Cl)c2-c2ccncn2)c1C(F)(F)F ; C[C@]1(O)C[C@@H](n2ncc(-c3onc(-c4c(F)cccc4Cl)c3-c3ccncn3)c2C(F)(F)F)C1]

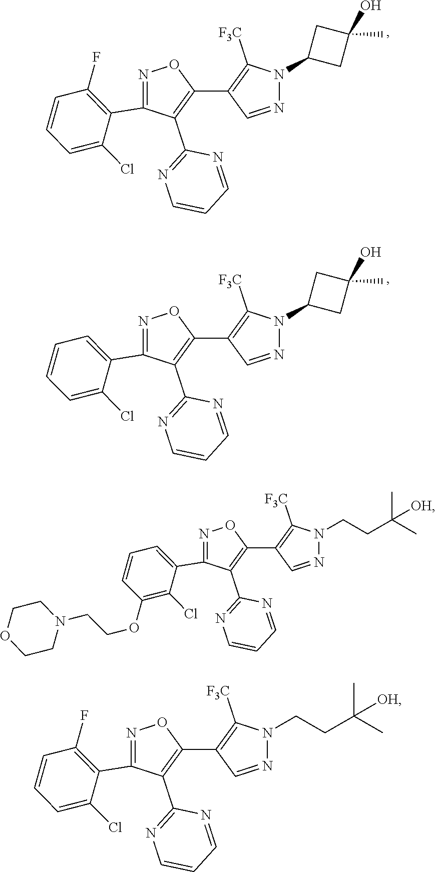

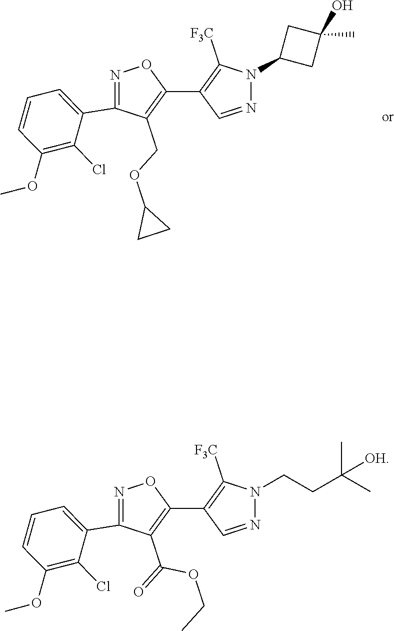

18. The method for treating a cancer in a subject in need thereof according to any one of items 14 to 17, wherein the cancer is selected from the group consisting of prostate cancer, breast cancer, lung cancer, ovarian cancer, bladder cancer, endometrial cancer, liver cancer, glioblastoma, B-cell lymphoma and colon cancer.
19. The method for treating a cancer in a subject in need thereof according to any one of items 14 to 18, wherein the cancer is resistant to a chemotherapeutic agent selected from tamoxifen, a taxane, an anthracenedione or combinations thereof.

In a particular embodiment of item 19, the taxane according to item 19 is selected from the group consisting of paclitaxel, docetaxel, cabazitaxel, hongdoushan A, hongdoushan B, hongdoushan C, baccatin I, baccatin II, 10-deacetylbaccatin or combinations thereof.
20. The method for treating a cancer in a subject in need thereof according to item 18 or 19, wherein the prostate cancer is a castration-resistant prostate cancer.

In a particular embodiment of item 20, the cancer according to item 18 is a non-small-cell lung cancer (NSCLC), K-Ras mutant lung cancer, BRAF mutant lung cancer, EGFR mutant lung cancer, tyrosine kinase inhibitor-resistant lung cancer or small cell lung cancer (SCLC).

In an additional particular embodiment of item 20, the breast cancer according to item 18 is a triple-negative breast cancer (TNBC), tamoxifen-resistant breast cancer, radiation-resistant breast cancer, HER2-positive breast cancer or ER-positive breast cancer.
21. The method according to any one of items 14 to 20, wherein the subject is a human in need of cancer treatment.
22. The method according to any one of items 14 to 21, wherein said compound according to Formula (I) is provided as a suitable pharmaceutical composition, such as a tablet, capsule, granule, powder, sachet, reconstitutable powder, dry powder inhaler and/or chewable.
23. The method according to any one of items 14 to 22, wherein said compound according to Formula (I) is administered to said subject in an effective dosage regimen, for example one to three times a day with a cumulative daily dose of 150 to 900 mg.
24. The method according to any one of items 14 to 23, wherein said method further comprises administering to the subject an effective amount of an anticancer drug.

In a particular embodiment of item 24, the anticancer drug is a chemotherapeutic agent selected from a taxane, an anthracenedione or combinations thereof.

In an additional particular embodiment of item 24, the anticancer drug is a taxane, wherein the taxane is selected from the group consisting of paclitaxel, docetaxel, cabazitaxel, hongdoushan A, hongdoushan B, hongdoushan C, baccatin I, baccatin II, 10-deacetylbaccatin or combinations thereof.

In an additional particular embodiment of item 24, the anticancer drug is an endocrine therapy selected from the group consisting of tamoxifen, raloxifene, an aromatase inhibitor, megestrol acetate, lasofoxifene, bazedoxifene, bazedoxifene/conjugated estrogens or combinations thereof.
25. The method according to item 24, wherein the compound according to Formula (I) enhances the therapeutic effect of the anticancer drug.
26. The method according to item 25, wherein the compound according to Formula (I) reverses or reduces cancer cell resistance to the anticancer drug and/or sensitizes cancer cells to the anticancer drug.
27. A kit comprising one or more compounds according to Formula (I) or a pharmaceutically acceptable salt, a solvate, a solvate of a salt, a hydrate or a polymorph thereof as defined in any one of items 1 to 26 and a label describing a method of administering one or more compounds according to Formula (I) or a pharmaceutically acceptable salt, a solvate, a solvate of a salt, a hydrate or a polymorph thereof and/or one or more anticancer drugs.

### DEFINITIONS AND PARTICULAR EMBODIMENTS

In particular embodiments, as used herein a heteroaryl group denotes a 5- or 6-membered heterocyclic group containing at least one heteroatom independently selected from O, N or S. This heterocyclic group is optionally fused to another aromatic or heteroaromatic 5- or 6-membered ring containing from one to four heteroatoms independently selected from O, N or S. For example, this group can be selected from a thiadiazole, thiazol-2-yl, thiazol-4-yl, thiazol-5-yl, isothiazol-3-yl, isothiazol-4-yl, isothiazol-5-yl, oxazol-2-yl, oxazol-4-yl, oxazol-5-yl, isooxazol-3-yl, isooxazol-4-yl, isooxazol-5-yl, 1,2,4-oxadiazol-3-yl, 1,2,4-oxadiazol-5-yl, 1,2,5-oxadiazol-3-yl, benzooxazol-2-yl, benzooxazol-4-yl, benzooxazol-5-yl, benzoisooxazol-3-yl, benzoisooxazol-4-yl, benzoisooxazol-5-yl, 1,2,5-oxadiazol-4-yl, 1,3,4-oxadiazol-2-yl, 1,2,4-thiadiazol-3-yl, 1,2,4-thiadiazol-5-yl, 1,3,4-thiadiazol-2-yl, isothiazol-3-yl, isothiazol-4-yl, isothiazol-5-yl, benzoisothiazol-3-yl, benzoisothiazol-4-yl, benzoisothiazol-5-yl, 1,2,5-thiadiazol-3-yl, 1-imidazolyl, 2-imidazolyl, 1,2,5-thiadiazol-4-yl, 4-imidazolyl, benzoimidazol-4-yl, 1-pyrrolyl, 2-pyrrolyl, 3-pyrrolyl, 2-firanyl, 3-firanyl, 2-thienyl, 3-thienyl, 2-pyridyl, 3-pyridyl, 4-pyridyl, 2-pyranyl, 3-pyranyl, 4-pyranyl, 2-pyrimidinyl, 4-pyrimidinyl, 5-pyrimidinyl, pyrid-2-yl, pyrid-3-yl, pyrid-4-yl, pyrid-5-yl, pyrid-6-yl, 3-pyridazinyl, 4-pyridazinyl, 2-pyrazinyl, 1-pyrazolyl, 3-pyrazolyl, 4-pyrazolyl, 1,2,3-triazol-4-yl, 1,2,3-triazol-5-yl, 1,2,4-triazol-3-yl, 1,2,4-triazol-5-yl, 1H-tetrazol-2-yl, 1H-tetrazol-3-yl, tetrazolyl, acridyl, phenazinyl, carbazolyl, phenoxazinyl, indolizine, 2-indolyl, 3-indolyl, 4-indolyl, 5-indolyl, 6-indolyl, 7-indolyl, 1-isoindolyl, 3-isoindolyl, 4-isoindolyl, 5-isoindolyl, 6-isoindolyl, 7-isoindolyl, 2-indolinyl, 3-indolinyl, 4-indolinyl, 5-indolinyl, 6-indolinyl, 7-indolinyl, benzo[b]furanyl, benzofurazane, benzothiofurazane, benzotriazol-1-yl, benzotriazol-4-yl, benzotriazol-5-yl, benzotriazol-6-yl, benzotriazol-7-yl, benzotriazine, benzo[b]thiophenyl, benzimidazolyl, benzothiazolyl, quinazolinyl, quinoxazolinyl, cinnoline, quinolinyl, tetrahydroquinolinyl, isoquinolinyl, or tetrahydroisoquinolinyl, purine, phthalazine, pteridine, thiatetraazaindene, thiatriazaindene, isothiazolopyrazine, 6-pyrimidinyl, 2,4-dimethoxy-6-pyrimidinyl, benzimidazol-2-yl, 1H-benzimidazolyl, benzimidazol-4-yl, benz-imidazol-5-yl, benzimidazol-6-yl, benzimidazol-7-yl, tetrazole, tetrahydro-thieno[3,4-d]imidazol-2-one, pyrazolo[5,1-c][1,2,4]triazine, isothiazolopyrimidine, pyrazolotriazine, pyrazolopyrimidine, imidazopyridazine, imidazopyrimidine, imidazopyridine, imidazolotriazine, triazolotriazine, triazolopyridine, triazolopyrazine, triazolopyrimidine, or triazolopyridazine group. Particular heteroaryl groups are pyrimidin-4-yl, pyrimidin-2-yl, thiazol-2-yl, pyrazin-2-yl and isoxazol-2-yl.

In particular embodiments, as used herein a heterocyclyl group denotes a 3- to 8-membered, more particularly a 3 to 6-membered heterocyclic non-aromatic group containing from one to four heteroatoms independently selected from the group consisting of O, N, and S, wherein the heterocyclyl group is optionally fused to another non-aromatic cycloalkyl or heterocyclyl ring; the heterocyclyl residue is in particular selected from the group consisting of oxetanyl, morpholine-4-yl, piperazinyl, isoxazolidine-2-yl, 1-alkylpiperazine-4-yl, pyrrolidinyl, pyrrolidino, piperidinyl, piperidino, piperazinyl, piperazino, morpholinyl, morpholino, thiomorpholinyl, thiomorpholino, tetrahydrofuranyl, tetrahydrothiofuranyl, tetrahydropyranyl and pyranyl. Particular heterocyclyl groups are tetrahydropyranyl and oxetanyl.

To keep the definitions as short as possible, as used herein “alkyl”, and “alk” (as e.g. in alkoxy) is to be understood to encompass linear and branched alkanyl, alkenyl and alkynyl, more particularly alkyl and alkenyl, even more particularly alkanyl. If not stated otherwise, these are in particular embodiments C₁₋₆-alkanyl, C₂₋₆-alkenyl or C₂₋₆-alkynyl, more particularly C₁₋₅-alkanyl, C₂₋₅-alkenyl or C₂₋₅-alkynyl, even more particularly C₁₋₄-alkanyl, C₂₋₄-alkenyl or C₂₋₄-alkynyl. The alkyl group may for instance be selected from the group consisting of —CH₃, —C₂H₅, —CH═CH₂, —C≡CH, —C₃H₇, —CH(CH₃)₂, —CH₂—CH═CH₂, —C(CH₃)=CH₂, —CH═CH—CH₃, —C≡C—CH₃, —CH₂—C≡CH, —C₄H₉, —CH₂—CH(CH₃)₂, —CH(CH₃)—C₂H₅, —C(CH₃)₃, —C₅H₁₁, —C₆H₁₃, —C₂H₄—CH═CH₂, —CH═CH—C₂H₅, —CH═C(CH₃)₂, —CH₂—CH═CH—CH₃, —CH═CH—CH═CH₂, —C₂H₄—C≡CH, —C≡C—C₂H₅, —CH₂—C≡C—CH₃, —C≡C—CH═CH₂, —CH═CH—C≡CH, —C≡C—C≡CH, —C₂H₄—CH(CH₃)₂, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —C₃H₆—CH═CH₂, —CH═CH—C₃H₇, —C₂H₄—CH═CH—CH₃, —CH₂—CH═CH—C₂H₅, —CH₂—CH═CH—CH═CH₂, —CH═CH—CH═CH—CH₃, —CH═CH—CH₂—CH═CH₂, —C(CH₃)=CH—CH═CH₂, —CH═C(CH₃)—CH═CH₂, —CH═CH—C(CH₃)=CH₂, —CH₂—CH═C(CH₃)₂, C(CH₃)═C(CH₃)₂, —C₃H₆—C≡CH, —C≡C—C₃H₇, —C₂H₄—C≡C—CH₃, —CH₂—C≡C—C₂H₅, —CH₂—C≡C—CH═CH₂, —CH₂—CH═CH—C≡CH, —CH₂—C≡C—C≡CH, —C≡C—CH═CH—CH₃, —CH═CH—C≡C—CH₃, —C≡C—C≡C—CH₃, —C≡C—CH₂—CH═CH₂, —CH═CH—CH₂—C≡CH, —C≡C—CH₂—C≡CH, —C(CH₃)=CH—CH═CH₂, —CH═C(CH₃)—CH═CH₂, —CH═CH—C(CH₃)=CH₂, —C(CH₃)=CH—C≡CH, —CH═C(CH₃)—C≡CH, —C≡C—C(CH₃)=CH₂, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —C₄H₈—CH═CH₂, —CH═CH—C₄H₉, —C₃H₆—CH═CH—CH₃, —CH₂—CH═CH—C₃H₇, —C₂H₄—CH═CH—C₂H₅, —CH₂—C(CH₃)═C(CH₃)₂, —C₂H₄—CH═C(CH₃)₂, —C₄H₈—C≡CH, —C≡C—C₄H₉, —C₃H₆—C≡C—CH₃, —CH₂—C≡C—C₃H₇ and —C₂H₄—C≡C—C₂H₅. Particular alkyl groups are methyl, ethyl, propyl, isopropyl, butyl, tert-butyl, pentyl, butenyl and pentenyl.

In particular embodiments, as used herein an arylalkyl group denotes a linear or branched C₁-C₆-alkyl substituted with at least one aryl group as defined herein. Exemplary arylalkyl groups include benzyl, phenylethyl, 4-hydroxybenzyl, 3-fluorobenzyl, 2-fluorophenylethyl and the like.

In particular embodiments, as used herein a cycloalkyl group denotes a non-aromatic ring system containing three to eight carbon atoms, particularly four to eight carbon atoms, more particularly three to six carbon atoms, even more particularly three to five carbon atoms.

In particular embodiments, as used herein an alkoxy group denotes an O-alkyl group, the alkyl group being as defined above. More particularly the alkoxy group is a methoxy, ethoxy, isopropoxy, tert-butoxy or pentoxy group, even more particularly methoxy.

In particular embodiments, as used herein a haloalkyl group denotes an alkyl group wherein one or more, particularly more than half, more particularly all, of the hydrogen atoms are replaced by halogen atoms.

The haloalkyl group is for instance —C(R¹⁰)₃, —CR¹⁰(R^{10′})₂, —CR¹⁰(R^{10′})R^{10″}, —C₂(R¹⁰)₅, —CH₂—C(R¹⁰)₃, —C(R^{10′})₂—CH(R^{10′})₂, —CH₂—CR¹⁰(R^{10′})₂, —CH₂—CR¹⁰(R^{10′})R^{10″}, —C₃(R¹⁰)₇, or —C₂H₄—C(R¹⁰)₃, wherein R¹⁰, R^{10′}, R^{10″} particularly independently represent F, Cl, Br or I, more particularly F. More particularly, haloalkyl is CF₃.

In particular embodiments, as used herein a haloalkoxy group denotes an —O-haloalkyl group.

In particular embodiments, as used herein a halo or halogen group denotes fluorine, chlorine, bromine or iodine; particularly chlorine or fluorine.

Unless stated otherwise, the terms “included”, “including”, “include” and the like are to be understood as meaning including but non-limiting.

Constituents which are optionally substituted as stated herein may be substituted, unless otherwise noted, at any chemically possible position.

In particular embodiments of the present invention, Ar is selected from the group consisting of phenyl and heteroaryl, each of which is optionally substituted by one or more substituents R′ independently selected from the group consisting of halogen, —OH, —CN, alkoxy, haloalkoxy, alkyl, haloalkyl, mono- or dialkylamino-alkyl, mono- or di-alkylamino-alkoxy, —COOR′, —CONHR′, —CO—R′, —SO₂NHR′, —NH—CO—R′, —NO₂, —NH—SO₂—R′, —SO₂—R′, benzyloxy, —CO-heterocyclyl, —CO-cycloalkyl, —CONH-cycloalkyl, —CONH— heterocyclyl, —O-alkyl-heterocyclyl, —O-alkyl-cycloalkyl, (2-oxa-6-azaspiro[3.3]hept-6-yl)—C₁₋₄-alkoxy, amino, arylalkyl, cycloalkyl, heterocyclyl, phenyl and heteroaryl, wherein each of said alkoxy, arylalkyl, alkyl, cycloalkyl, heterocyclyl, phenyl and heteroaryl groups is optionally substituted be one or more substituents independently selected from alkyl, haloalkyl, halogen and OH, and R′ is independently selected from the group consisting of H, OH, alkyl and haloalkyl;
in other particular embodiments Ar is phenyl, which is optionally substituted by one or more substituents independently selected from fluoro, chloro, alkoxy, fluoroalkoxy, —CO-heterocyclyl and —O-alkyl-heterocyclyl;
in other particular embodiments Ar is phenyl, which is optionally substituted by one or more independently selected from fluoro, chloro, —OMe, —OCHF₂, —OCF₃, —CO-heterocyclyl and —O—C₁₋₂-alkyl-heterocyclyl;
in other particular embodiments Ar is selected from

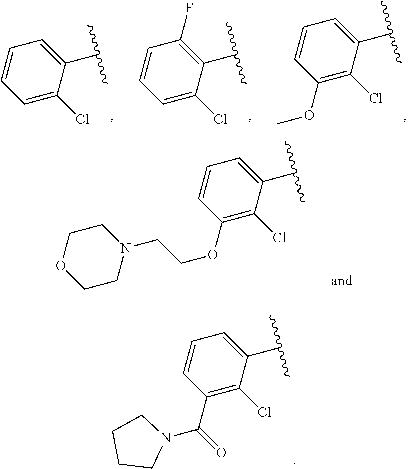

In particular embodiments of the present invention, Z is selected from the group consisting of H, halogen, —CO—R^{Z}, —CH₂—O—R^{Z}, —CO—CH₂—R^{Z}, —CO—CH₂—O—R^{Z}, —COOR^{Z}, —NHCO—R^{Z}, —CO—NHR^{Z}, —N(R^{Z})₂, —CN, —NHCOOR^{Z}, —SO₂—R^{Z}, —SO₂NHR^{Z}, -alkyl-O—R^{Z}, -alkyl-O-alkyl-O—R, amino, alkyl, phenyl, heteroaryl, heterocyclyl and cycloalkyl, wherein each of said alkyl, phenyl, heteroaryl, heterocyclyl and cycloalkyl groups is optionally substituted by one or more substituents independently selected from the group consisting of halogen, alkyl, alkoxy, haloalkyl, —COO-alkyl, OH and cycloalkyl, and R^{Z} is selected from the group consisting of H, halogen, —OH, alkyl, haloalkyl, cycloalkyl, heterocyclyl, phenyl and heteroaryl;
in other particular embodiments Z is selected from the group consisting of —CO—R^{Z}, —CH₂—O—R^{Z} and heteroaryl, wherein said heteroaryl is optionally substituted by one or more substituents independently selected from the group consisting of fluoro, chloro, alkyl, alkoxy, fluoroalkyl and OH, and R^{Z} is selected from the group consisting of alkyl, fluoroalkyl and cycloalkyl, heterocyclyl;
in other particular embodiments Z is selected from the group consisting of —CO—C₁₋₃-alkyl, —CH₂—O—C₃₋₄-cycloalkyl, thiazolyl and pyrimidyl;
in other particular embodiments Z is selected from

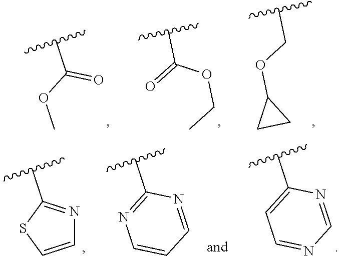

In particular embodiments of the present invention, Y is selected from H, halogen, haloalkyl, alkyl or an alkylester;
in other particular embodiments Y is selected from alkyl or fluoroalkyl;
in other particular embodiments Y is selected from methyl, CHF₂ and CF₃;
in other particular embodiments Y is selected from methyl and CF₃;
in other particular embodiments Y is CF₃.

In particular embodiments of the present invention, R¹ is selected from aryl, heteroaryl, heterocyclyl or alkyl, which can be substituted by one or more substituents R″ independently selected from H, —CO₂R′″, —CONHR′″, —CR′″O, —SO₂N(R′″)₂, —SO₂NHR′″, —NR′″—CO-haloalkyl, —NO₂, —NR′″—SO₂-haloalkyl, —NR′″—SO₂-alkyl, —SO₂-alkyl, —NR′″—CO-alkyl, —CN, alkyl, cycloalkyl, aminoalkyl, alkylamino, alkoxy, —OH, —SH, alkylthio, hydroxyalkyl, hydroxyalkylamino, halogen, haloalkyl, haloalkoxy, amino, heterocyclyl, aryl, haloaryl, haloarylalkyl, arylalkyl or heteroaryl, wherein the longest chain allowed in R¹ are three coupled substituents R″ and/or R′″, and with R′″ independently represents H, haloalkyl, hydroxyalkyl, amino, alkoxy, —N═C(R″)₂, —NR″—CO—R″, —CR″O, —CO₂R″, alkyl, cycloalkyl, aryl, haloaryl, haloarylalkyl, heteroaryl, heterocyclyl, arylalkyl or aminoalkyl, which are optionally substituted by one or more substituents R″,
Or alternatively R′ is a group of the structure

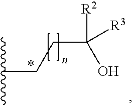

wherein
n is 0 or 1, R² is H, deuterium or methyl, R³ is methyl, trifluoromethyl, ethyl, or taken with R² together forms a cyclopropyl group, or
n is 1, R² is H, deuterium or methyl and R³ forms a methylene bridge to the carbon atom marked *; in other particular embodiments R′ is selected from aryl, which can be substituted by one or more substituents selected from chloro, fluoro, —CN, alkyl, fluoroalkyl, alkoxy or fluoroalkoxy;
in other particular embodiments R¹ is

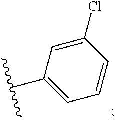 [SMILES: *c1cccc(Cl)c1]

in other particular embodiments R¹ is a structure

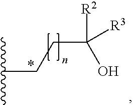

wherein
n is 0 or 1, R² is H, deuterium or methyl, R³ is methyl, trifluoromethyl, ethyl, or taken with R² together forms a cyclopropyl group;
in other particular embodiments R¹ is a structure

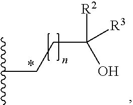

wherein
n is 1, R² is H, deuterium or methyl and R³ forms a methylene bridge to the carbon atom marked *;
in other particular embodiments R¹ is

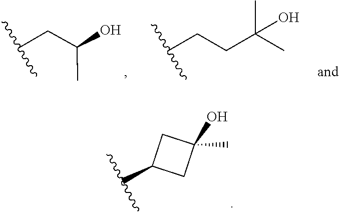

Particular compounds of the present invention are the compounds of the below examples of the present invention.

The term “pharmaceutically acceptable salts” refers to salts prepared from pharmaceutically acceptable non-toxic bases, including inorganic bases and organic bases. Thus, the compounds of the present disclosure which contain acidic groups can be present on these groups and can be used according to the disclosure, for example, as alkali metal salts, alkaline earth metal salts or ammonium salts. More precise examples of such salts include sodium salts, potassium salts, calcium salts, magnesium salts or salts with ammonia or organic amines such as, for example, ethylamine, ethanolamine, triethanolamine or amino acids. The respective salts can be obtained by customary methods which are known to the person skilled in the art like, for example, by contacting these with an organic or inorganic base in a solvent or dispersant, or by cation exchange with other salts. The present disclosure also includes all salts of the compounds of the present disclosure which, owing to low physiological compatibility, are not directly suitable for use in pharmaceuticals but which can be used, for example, as intermediates for chemical reactions or for the preparation of pharmaceutically acceptable salts.

The term “solvate” refers to a crystalline form of a molecule that further comprises molecules of a solvent or solvents incorporated into the crystalline lattice structure. Thus, the compounds of the present disclosure may be present in the form of solvates, such as those which include as solvate water, or pharmaceutically acceptable solvates, such as alcohols, in particular ethanol. A stoichiometric or non-stoichiometric amount of solvent is bound by non-covalent intermolecular forces. When the solvent is water, the “solvate” is a “hydrate.” It is understood, that a “pharmaceutically acceptable salts” can in addition optionally contain a “solvate”.

The term “polymorph” as used herein refers to a crystalline form of a compound or a salt, hydrate, or solvate thereof, in a particular crystal packing arrangement. All polymorphs have the same elemental composition. The term “crystalline” as used herein, refers to a solid state form which consists of orderly arrangement of structural units. Different crystalline forms of the same compound, or a salt, hydrate, or solvate thereof, arise from different packing of the molecules in the solid state, which results in different crystal symmetries and/or unit cell parameter. Different crystalline forms usually have different X-ray diffraction patterns, infrared spectra, melting points, density, hardness, crystal shape, optical and electrical properties, stability and solubility.

As used herein, the term “subject” refers to any member of the animal kingdom including humans. In some embodiments, “subject” refers to humans, at any stage of development. In some embodiments, “subject” refers to a human patient. In some embodiments, “subject” refers to non-human animals. In some embodiments, the non-human animal is a mammal (e.g. a rodent, a mouse, a rat, a rabbit, a monkey, a dog, a cat, a sheep, cattle, a primate or a pig). In some embodiments, subjects include, but are not limited to, mammals, birds, reptiles, amphibians, fish or worms. In some embodiments, a subject may be a transgenic animal, genetically-engineered animal or a clone.

As used herein, the term “effective amount” includes a dosage sufficient to produce a desired result with respect to the indicated disorder, condition or mental state. The desired result may comprise a subjective or objective improvement in the recipient of the dosage. In one non-limiting example, an effective amount of a compound according to Formula (I) includes an amount sufficient to alleviate the signs, symptoms or causes of a cancer such as prostate cancer, e.g. CRPC. Thus, an effective amount can be an amount that slows or reverses tumor growth, increases mean time of survival, inhibits tumor progression or metastasis, or sensitizes a cancer cell to an anticancer drug to which it has become or is resistant. Also, in a second non-limiting example, an effective amount of a compound according to Formula (I) includes an amount sufficient to cause a substantial improvement in a subject having cancer when administered to the subject. The amount will vary with the type of cancer being treated, the stage of advancement of the cancer, the type and concentration of composition applied, and the amount of anticancer drug (e.g. anti-androgen drug) that is also administered to the subject. In a third non-limiting example, an effective amount of a compound according to Formula (I) can include an amount that is effective in enhancing the therapeutic activity of anticancer drugs such as anti-androgen drugs (e.g. bicalutamide, enzalutamide, arbiraterone, etc.) and/or chemotherapeutic agents (e.g. tamoxifen and/or taxanes such as docetaxel).

As used herein, the term “treating” includes, but is not limited to, methods and manipulations to produce beneficial changes in a recipient's health status, e.g. a patient's cancer status. The changes can be either subjective or objective and can relate to features such as symptoms or signs of the cancer being treated. For example, if the patient notes decreased pain, then successful treatment of pain has occurred. For example, if a decrease in the amount of swelling has occurred, then a beneficial treatment of inflammation has occurred. Similarly, if the clinician notes objective changes, such as reducing the number of cancer cells, the growth of the cancer cells, the size of cancer tumors or the resistance of the cancer cells to another cancer drug, then treatment of cancer has also been beneficial. Preventing the deterioration of a recipient's status is also included by the term. Treating, as used herein, also includes administering a compound according to Formula (I) alone or in combination with an anticancer drug to a subject having cancer. In certain instances, the cancer is prostate cancer, lung cancer, breast cancer, liver cancer, ovarian cancer, endometrial cancer, bladder cancer, colon cancer, lymphoma or glioblastoma multiforme.

As used herein, the term “administering” includes activities associated with providing a patient an amount of a compound described herein, e.g. a compound according to Formula (I). Administering includes providing unit dosages of compositions set forth herein to a patient in need thereof.

Administering includes providing effective amounts of compounds, e.g. a compound according to Formula (I), for a specified period of time, e.g. for about 1, 2, 3, 4, 5, 6, 7, 8, 9, 10, 12, 13, 14, 15, 16, 17, 18, 19, 20, 21, 22, 23, 24, 25, 26, 27, 28, 29, 30, 31 or more days, 1, 2, 3 or more months, or in a specified sequence, e.g. administration a compound according to Formula (I) followed by the administration of one or more anticancer drugs, or vice versa.

As used herein, the term “co-administering” includes sequential or simultaneous administration of two or more structurally different compounds. For example, two or more structurally different pharmaceutically active compounds can be co-administered by administering a pharmaceutical composition adapted for oral administration that contains two or more structurally different active pharmaceutically active compounds. As another example, two or more structurally different compounds can be co-administered by administering one compound and then administering the other compound. In some instances, the co-administered compounds are administered by the same route. In other instances, the co-administered compounds are administered via different routes. For example, one compound can be administered orally, and the other compound can be administered, e.g. sequentially or simultaneously, via intravenous or intraperitoneal injection.

As used herein, the term “cancer” refers to conditions including solid cancers, lymphomas and leukemias. Examples of different types of cancers include, but are not limited to, prostate cancer, lung cancer (e.g. non-small cell lung cancer or NSCLC), ovarian cancer, colorectal cancer, liver cancer (i.e. hepatocarcinoma), renal cancer (i.e. renal cell carcinoma). bladder cancer, breast cancer, thyroid cancer, pleural cancer, pancreatic cancer, uterine cancer, cervical cancer, testicular cancer, anal cancer, bile duct cancer, gastrointestinal carcinoid tumors, esophageal cancer, gall bladder cancer, appendix cancer, small intestine cancer, stomach (gastric) cancer, cancer of the central nervous system, skin cancer, choriocarcinoma, head and neck cancer, blood cancer, endometrial cancer, osteogenic sarcoma, fibrosarcoma, neuroblastoma, glioma, melanoma, B-cell lymphoma, non-Hodgkin's lymphoma, Burkitt's lymphoma, Small Cell lymphoma, Large Cell lymphoma, monocytic leukemia, myelogenous leukemia, acute lymphocytic leukemia, acute myelocytic leukemia and multiple myeloma. In some instances, the cancer can be metastatic. In certain instances, the cancer is prostate cancer, lung cancer, breast cancer, liver cancer, ovarian cancer, endometrial cancer, bladder cancer, colon cancer, lymphoma or a glioma such as glioblastoma multiforme. In other instances, the cancer can be resistant to an anticancer drug, e.g. an anti-androgen-resistant cancer, a taxane-resistant cancer (e.g. docetaxel-resistant cancer), a tamoxifen-resistant cancer, a radiation-resistant cancer or a tyrosine kinase inhibitor-resistant cancer.

As used herein, the terms “RORγ-dependent cancer(s)” or “RORγ-sensitive cancer(s)” refer to cancer(s) that involve IL-23-responsive, RORγ/RORγt and IL-17-expressing cells. Examples of RORγ dependent cancers include prostate cancer, breast cancer, particularly Triple-Negative Breast Cancer (TNBC), pancreatic cancer, gastric cancer, hepatocellular carcinoma (HCC), lung cancer, liver cancer, ovarian cancer, endometrial cancer, bladder cancer, colon cancer, lymphoma and glioma.

As used herein, the terms “prostate cancer” or “prostate cancer cell” refer to a cancer cell or cells that reside in prostate tissue. The prostate cancer can be benign, malignant or metastatic. The prostate cancer can be androgen-insensitive, hormone-resistant or castrate-resistant. The prostate cancer can be “advanced stage prostate cancer” or “advanced prostate cancer”. Advanced stage prostate cancer includes a class of prostate cancers that has progressed beyond early stages of the disease. Typically, advanced stage prostate cancers are associated with a poor prognosis. Types of advanced stage prostate cancers include, but are not limited to, metastatic prostate cancer, drug-resistant prostate cancer such as anti-androgen-resistant prostate cancer (e.g. enzalutamide-resistant prostate cancer, abiraterone-resistant prostate cancer, bicalutamide-resistant prostate cancer, etc.), taxane-resistant prostate cancer (e.g. docetaxel-resistant prostate cancer) and the like, hormone refractory prostate cancer, castration-resistant prostate cancer (CRPC), metastatic castration-resistant prostate cancer, AR-V7-induced drug-resistant prostate cancer such as AR-V7-induced anti-androgen-resistant prostate cancer (e.g. AR-V7-induced enzalutamide-resistant prostate cancer), AKR1C3-induced drug-resistant prostate cancer such as AKR1C3-induced anti-androgen-resistant prostate cancer (e.g. AKR1C3-induced enzalutamide-resistant prostate cancer) and combinations thereof. In some instances, the advanced stage prostate cancers do not generally respond, or are resistant, to treatment with one or more of the following conventional prostate cancer therapies: enzalutamide, abiraterone, bicalutamide and docetaxel. Compounds, compositions, and methods of the present invention are provided for treating prostate cancer, such as advanced stage prostate cancer, including any one or more (e.g., two, three, four, five, six, seven or more) of the types of advanced stage prostate cancers disclosed herein.

As used herein, the phrase “enhancing the therapeutic effects” includes any of a number of subjective or objective factors indicating a beneficial response or improvement of the condition being treated as discussed herein. For example, enhancing the therapeutic effects of an anticancer drug such as an anti-androgen drug (e.g. enzalutamide, abiraterone or bicalutamide) or a chemotherapeutic agent such as tamoxifen or a taxane (e.g. docetaxel) includes reversing or reducing cancer cell resistance and/or sensitizing a drug-resistant cancer to anticancer drug therapy. Also, for example, enhancing the therapeutic effects of an anticancer drug includes altering drug-resistant cancer cells so that the cells are not resistant to the anticancer drug. Also, for example, enhancing the therapeutic effects of an anticancer drug includes additively or synergistically improving or increasing the activity of the anticancer drug. In some embodiments, the enhancement includes a one-fold, two-fold, three-fold, five-fold, ten-fold, twenty-fold, fifty-fold, hundred-fold, or thousand-fold increase in the therapeutic activity of an anticancer drug used to treat cancer.

As used herein, the phrase “reversing cancer cell resistance” includes altering or modifying a cancer cell that is resistant to anticancer drug therapy so that the cell is no longer resistant to anticancer drug therapy. As used herein, the phrase “reducing cancer cell resistance” includes increasing the therapeutic activity of an anticancer drug towards cancer cells that are, or previously were, resistant to anticancer drug therapy.

As used herein, the phrase “sensitizing cancer cell resistance” includes inducing sensitization towards anticancer drug therapy in cancer cells which are resistant to anticancer drug therapy. Sensitization as used herein includes inducing the ability of a cancer cell to be effectively treated with an anticancer drug. Sensitization also includes reducing the dosage required to achieve a beneficial effect with an anticancer drug.

As used herein, the phrase “anti-androgen drug” includes anti-androgen compounds that alter the androgen pathway by blocking the androgen receptors, competing for binding sites on the cell's surface, or affecting or mediating androgen production. Anti-androgen drugs are useful for treating several diseases including, but not limited to, prostate cancer. Anti-androgen drugs include, but are not limited to, enzalutamide, abiraterone, bicalutamide, flutamide, nilutamide, apalutamide, finasteride, dutasteride, alfatradiol and combinations thereof.

As used herein, the term “androgen receptor” or “AR” includes a nuclear receptor that binds androgenic hormones testosterone or dihydrotestosterone in the cytoplasm and translocates to the nucleus. AR modulates, inter alia, transcription of target genes by binding to Androgen Response Elements (AREs) in the promoters of such target genes.

As used herein, the term “AR variant” includes a splice variant of full-length AR. Various AR variants are known. See, Guo et al., Cancer Res. 2009; 69:2305. Exemplary AR variants include, but are not limited to, variants lacking a functional ligand binding domain (LBD). An example of an AR variant that lacks an LBD is AR-V7. “AR-V7” includes androgen receptor splice variant 7, a constitutively active variant of an AR that lacks a functional ligand binding domain (LBD). See, e.g. Hu et al., Cancer Res. 2009; 69:16.

### Anticancer Drugs

In certain embodiments, the compounds according to Formula (I) of the present invention can be used in combination with an anticancer drug to reduce or reverse cancer cell resistance to the anticancer drug by sensitizing the cancer cell to the anticancer drug.

Non-limiting examples of anticancer drugs include anti-androgen drugs, chemotherapeutic agents, radiotherapeutic agents, antigen-specific immunotherapeutic agents, endocrine therapies, tyrosine kinase inhibitors and combinations thereof.

### 1. Anti-Androgen Drugs

Anti-androgen drugs are compounds that inhibit the transcription, translation, stability and/or activity of androgen receptors (AR) or variants thereof (e.g. AR-V7). Inhibition of AR activity can include inhibition of recruitment of AR to Androgen Response Elements (AREs). In some embodiments, inhibition of AR activity can include inhibition of recruitment of AR to the prostate specific antigen (PSA) promoter. In some embodiments, inhibition of AR activity can include inhibition of AR-induced activation of the PSA promoter. In some embodiments, inhibition of AR activity can include inhibition of AR-induced PSA production. For example, inhibition of AR can include inhibition of production of PSA in the absence of dihydrotestosterone.

Anti-androgen drugs include, but are not limited to, enzalutamide, abiraterone, bicalutamide, flutamide, nilutamide, apalutamide, finasteride, dutasteride, alfatradiol and combinations thereof.

In some embodiments, the present invention provides a composition comprising one or more compounds according to Formula (I) in combination with one or more anti-androgen drugs and it application in a method of treating a cancer in a subject in need thereof. In certain instances, the composition further comprises a pharmaceutically acceptable excipient or diluent. In other instances, the composition is formulated for oral or parenteral administration.

In other embodiments, the present invention provides a method for treating cancer in a subject comprising administering to the subject an effective amount of one or more compounds according to Formula (I) in combination with one or more anti-androgen drugs. In certain instances, the effective amount of one or more compounds according to Formula (I) is an amount sufficient to sensitize an anti-androgen drug-resistant cancer such as anti-androgen drug-resistant prostate cancer (e.g. castration-resistant prostate cancer) to anti-androgen drug treatment. The compounds according to Formula (I) and anti-androgen drugs can be delivered to a subject via the same route of administration (e.g. orally or parenterally) or via different routes of administration (e.g. intravenously for compounds according to Formula (I) and orally for anti-androgen drugs, or vice versa).

### 2. Chemotherapeutic Agents

Chemotherapeutic agents are well known in the art and include, but are not limited to, anthracenediones (anthraquinones) such as anthracyclines (e.g. daunorubicin (daunomycin; rubidomycin), doxorubicin, epirubicin, idarubicin and valrubicin), mitoxantrone and pixantrone; platinum-based agents (e.g. cisplatin, carboplatin, oxaliplatin, satraplatin, picoplatin, nedaplatin, triplatin and lipoplatin); tamoxifen and metabolites thereof such as 4-hydroxytamoxifen (afimoxifene) and N-desmethyl-4-hydroxytamoxifen (endoxifen); taxanes such as paclitaxel (taxol), docetaxel, cabazitaxel, hongdoushan A, hongdoushan B, hongdoushan C, baccatin I. baccatin II and 10-deacetylbaccatin; alkylating agents (e.g. nitrogen mustards such as mechlorethamine, cyclophosphamide, ifosfamide, melphalan (L-sarcolysin) and chlorambucil); ethylenimines and methylmelamines (e.g. hexamethylmelamine, thiotepa, alkyl sulphonates such as busulfan, nitrosoureas such as carmustine (BCNU), lomustine (CCNLJ), semustine (methyl-CCN-U) and streptozoein (streptozotocin), and triazenes such as decarbazine (DTIC; dimethyltriazenoimidazolecarboxamide)); antimetabolites (e.g. folic acid analogues such as methotrexate (amethopterin), pyrimidine analogues such as fluorouracil (5-fluorouracil; 5-FU), floxuridine (fluorodeoxyuridine; FUdR) and cytarabine (cytosine arabinoside), and purine analogues and related inhibitors such as mercaptopurine (6-mercaptopurine; 6-MP), thioguanine (6-thioguanine; 6-TG) and pentostatin (2′-deoxycofonnycin)); natural products (e.g. vinca alkaloids such as vinblastine (VLB) and vincristine, epipodophyllotoxins such as etoposide and teniposide, and antibiotics such as dactinomycin (actinomycin D), bleomycin, plicamycin (mithramycin) and mitomycin (mitomycin Q); enzymes such as L-asparaginase; biological response modifiers such as interferon alpha; substituted ureas such as hydroxyurea; methyl hydrazine derivatives such as procarbazine (N-methylhydrazine; MIH); adrenocortical suppressants such as mitotane (o,p′-DDD) and aminoglutethimide; analogs thereof; derivatives thereof; and combinations thereof.

In some embodiments, the present invention provides a composition comprising one or more compounds according to Formula (I) in combination with one or more chemotherapeutic agents and its application in a method of treating a cancer in a subject in need thereof. In certain instances, the composition further comprises a pharmaceutically acceptable excipient or diluent. In other instances, the composition is formulated for oral or parenteral administration.

In other embodiments, the present invention provides a method for treating cancer in a subject comprising administering to the subject an effective amount of one or more compounds according to Formula (I) in combination with one or more chemotherapeutic agents. In certain instances, the effective amount of one or more compounds according to Formula (I) is an amount sufficient to sensitize a chemotherapy drug-resistant cancer such as a tamoxifen-resistant cancer (e.g. tamoxifen-resistant breast cancer) or a taxane-resistant cancer (e.g. docetaxel-resistant prostate cancer) to chemotherapy drug treatment. The compounds according to Formula (I) and chemotherapeutic agents can be delivered to a subject via the same route of administration (e.g. orally or parenterally) or via different routes of administration (e.g. intravenously for compounds according to Formula (I) and orally for chemotherapeutic agents, or vice versa).

### 3. Radiotherapeutic Agents

Radiotherapeutic agents are well known in the art and can comprise external-beam radiation therapy and/or internal radiation therapy. External beam radiation therapy delivers radioactive beams of high energy X-rays and/or gamma rays to a patient's tumor, whereas internal radiation therapy delivers radioactive atoms to a patient's tumor. Both external beam radiation therapy and internal radiation therapy are used to suppress tumor growth or kill cancer cells by delivering a sufficient quantity of radioactivity to the target site. In some embodiments, the radiotherapeutic agent comprises a radioactive atom and is complexed with a biologic or synthetic agent to increase delivery to the target site. Such biologic or synthetic agents are known in the art. Suitable radioactive atoms for use with the compounds according to Formula (I) of the present invention include any of the radionuclides described herein, or any other isotope which emits enough energy to destroy a targeted tissue or cell. In some embodiments, radiotherapeutic agents may be coupled to targeting moieties, such as antibodies, to improve the localization of radiotherapeutic agents to cancerous cells.

The term “radionuclide” is intended to include any nuclide that exhibits radioactivity. A “nuclide” refers to a type of atom specified by its atomic number, atomic mass, and energy state, such as carbon 14 (¹⁴C). “Radioactivity” refers to the radiation, including alpha particles, beta particles, nucleons, electrons, positrons, neutrinos and gamma rays, emitted by a radioactive substance. Examples of radionuclides suitable for use in the present invention include, but are not limited to, fluorine 18 (¹⁸F), fluorine 19 (¹⁹F), phosphorus 32 (³²P), scandium 47 (⁴⁷Sc), cobalt 55 (⁵⁵Co), copper 60 (⁶⁰Cu), copper 61 (⁶¹Cu), copper 62 (⁶²Cu), copper 64 (⁶⁴Cu), gallium 66 (⁶⁶Ga), copper 67 (⁶⁷Cu), gallium 67 (⁶⁷Ga), gallium 68 (⁶⁸Ga), rubidium 82 (⁸²Rb), yttrium 86 (⁸⁶Y), yttrium 87 (⁸⁷Y), strontium 89 (⁸⁹Sr), yttrium 90 (⁹⁰Y), rhodium 105 (¹⁰⁵Rh), silver 111 (¹¹¹Ag), indium 111 (¹¹¹In), iodine 124 (¹²⁴I), iodine 125 (¹²⁵I), iodine 131 (¹³¹I), tin 117m (^{117m}Sn), technetium 99m (^{99m}Tc), promethium 149 (¹⁴⁹Pm), samarium 153 (⁵³Sm), holmium 166 (¹⁶⁶Ho), lutetium 177 (¹⁷⁷Lu), rhenium 186 (¹⁸⁶Re), rhenium 188 (¹⁸⁸Re), thallium 201 (²⁰¹Tl), astatine 211 (²¹¹At), bismuth 212 (²¹²Bi) and radium-223 (²²³Ra). As used herein, the “m” in ^{117m}Sn and ^{99m}Tc stands for the meta state. Additionally, naturally-occurring radioactive elements such as uranium, radium and thorium, which typically represent mixtures of radioisotopes, are suitable examples of radionuclides.

In some embodiments, the present invention provides a composition comprising one or more compounds according to Formula (I) in combination with one or more radiotherapeutic agents and its application in a method of treating a cancer in a subject in need thereof. In certain instances, the composition further comprises a pharmaceutically acceptable excipient or diluent. In other instances, the composition is formulated for oral or parenteral administration.

In other embodiments, the present invention provides a method for treating cancer in a subject comprising administering to the subject an effective amount of one or more compounds according to Formula (I) in combination with one or more radiotherapeutic agents. In certain instances, the effective amount of one or more compounds according to Formula (I) is an amount sufficient to sensitize a radiation-resistant cancer such as a radiation-resistant breast cancer to radiation treatment. The compounds according to Formula (I) and radiotherapeutic agents can be delivered to a subject via the same route of administration (e.g. orally or parenterally) or via different routes of administration.

### 4. Endocrine Therapies

Endocrine therapy is the manipulation of the endocrine system through the administration of specific hormones or drugs which inhibit or decrease the production or activity of targeted hormones or alter the gene expression pattern of targeted cells. Endocrine therapy is particularly useful in certain types of cancer, including breast cancer. Any known hormone antagonist or modulator may be used in the present invention. Endocrine therapies useful in the present invention include, but are not limited to, aromatase inhibitors (e.g. letrozole), megestrol acetate, flutamide, tamoxifen, raloxifene, lasofoxifene, bazedoxifene, bazedoxifene/conjugated estrogens, and combinations thereof.

In some embodiments, the present invention provides a composition comprising one or more compounds according to Formula (I) in combination with one or more endocrine therapies and its application in a method of treating a cancer in a subject in need thereof. In certain instances, the composition further comprises a pharmaceutically acceptable excipient or diluent. In other instances, the composition is formulated for oral or parenteral administration.

In other embodiments, the present invention provides a method for treating cancer in a subject comprising administering to the subject an effective amount of one or more compounds according to Formula (I) in combination with one or more endocrine therapies. In certain instances, the effective amount of one or more compounds according to Formula (I) is an amount sufficient to sensitize an endocrine therapy-resistant cancer such as a tamoxifen-resistant breast cancer to endocrine therapy. The compounds according to Formula (I) and endocrine therapies can be delivered to a subject via the same route of administration (e.g. orally or parenterally) or via different routes of administration.

### 5. Tyrosine Kinase Inhibitors

Tyrosine kinase inhibitors are small molecules that inhibit tyrosine kinase proteins. Tyrosine kinases are enzymes that activate many proteins in cellular signal transduction cascades by addition of a phosphate group to the protein. High expression and aberrant activation, of tyrosine kinase proteins can cause undesirable “switching on” of cellular signaling pathways that can result in uncontrolled cellular proliferation associated with cancerous cellular phenotypes. Various forms of cancer are currently treated by inhibiting or reducing the activity of poorly regulated tyrosine kinase proteins with tyrosine kinase inhibitors. Treatment regimens with tyrosine kinase inhibitors can suppress, reduce the incidence, reduce the severity, or inhibit the progression of cancer. Examples of tyrosine kinase inhibitors include, but are not limited to, gefitinib, erlotinib, sorafenib, sunitinib, dasatinib, lapatinib, nilotinib, bortezomib, salinomycin and combinations thereof.

In some embodiments, the present invention provides a composition comprising one or more compounds according to Formula (I) in combination with one or more tyrosine kinase inhibitors and its application in a method of treating a cancer in a subject in need thereof. In certain instances, the composition further comprises a pharmaceutically acceptable excipient or diluent. In other instances, the composition is formulated for oral or parenteral administration.

In other embodiments, the present invention provides a method for treating cancer in a subject comprising administering to the subject an effective amount of one or more compounds according to Formula (I) in combination with one or more tyrosine kinase inhibitors. In certain instances, the effective amount of one or more compounds according to Formula (I) is an amount sufficient to sensitize a tyrosine kinase inhibitor-resistant cancer such as a tyrosine kinase inhibitor-resistant non-small-cell lung cancer (NSCLC) to tyrosine kinase inhibitor therapy. The compounds according to Formula (I) and tyrosine kinase inhibitors can be delivered to a subject via the same route of administration (e.g., orally or parenterally) or via different routes of administration.

### 6. Antigen-Specific Immunotherapeutic Agents

In some embodiments, antigen-specific immunotherapeutic agents include compounds and compositions designed to stimulate the immune system to specifically recognize antigens expressed or overexpressed by cancerous cells. In other embodiments, antigen-specific immunotherapeutic agents include compounds and compositions that will specifically recognize antigens expressed or overexpressed by cancerous cells. Non-limiting examples of antigen-specific immunotherapeutic agents include vaccines (e.g. peptide vaccines), antibodies, cytotoxic T cell lymphocytes (CTLs), chimeric antigen receptor T cells (CAR-T cells), immune checkpoints (e.g. CTLA-4, PD-1 and PD-L1), immune modulating cytokines (e.g. IL-6 and IL-17) and combinations thereof. In particular embodiments, the antigens presented by cancerous cells are highly specific to each cancer type, and the vaccines, antibodies, CTLs and/or CAR-T cells used is dependent on the cancer type being treated.

A vaccine can stimulate the immune system to specifically recognize and attack antigens presented by cancerous cells. Vaccines can comprise one or more peptides, peptide fragments, fusion peptides, DNA, RNA, other biologic or non-biologic material or combinations thereof.

In some embodiments, one or more peptides, peptide fragments, or fusion peptides may be used for a peptide vaccine. The peptides may be harvested from an endogenous source or chemically synthesized.

The peptides chosen are specific for the type of cancer being treated. For example, when targeting cancer cells, some commonly targeted proteins include GM-CSF, IL-13Rα2, EphA2 and Survivin; however, specific cancer types will have specifically preferred peptides used for targeting afflicted cells. In some embodiments, the one or more peptides in the peptide vaccine are free soluble peptides. In other embodiments, the one or more peptides in the peptide vaccine are tethered together using any means known in the art.

In some embodiments, vaccines include cancer vaccines such as, e.g. tecemotide (L-BLP25), oncophage, sipuleucel-T and combinations thereof.

Antibodies can recognize antigens expressed or overexpressed by cancerous cells. Antigens recognized by these antibodies can be proteins expressed, activated, or overexpressed on the cell surface or proteins secreted into the extracellular fluid. In some embodiments, antibodies can be used to target human effector cells (e.g. macrophages) against the cancerous cells. In some embodiments, antibodies are used to inhibit the normal function of cell surface receptors. In some embodiments, antibodies bind to the ligands of cell surface receptors to block the cellular signaling cascade. Antibodies used as antigen-specific immunotherapeutic agents can be monoclonal or polyclonal antibodies as well as chimeric, humanized or human antibodies, and can be previously isolated from the patient or produced from another biologic source. Methods of producing antibodies are well known in the art, and may be made by any known means. For example, antibodies described herein can be produced by conventional monoclonal antibody methodology e.g. the standard somatic cell hybridization technique of Kohler and Milstein, Nature 1975; 256:495, the contents of which are herein incorporated by reference for all purposes. In some embodiments, antibodies useful in the treatment of cancer include immune checkpoint inhibitors. In particular embodiments, antibodies useful in the treatment of cancer include, but are not limited to, alemtuzumab, bevacizumab, cetuximab, ipilimumab, nivolumab, ofatumumab, panitumumab, pembrolizumab, atezolizumab, rituximab, trastuzumab and combinations thereof.

The use of CTLs and CAR-T cells as antigen-specific immunotherapeutic agents is a form of adoptive T cell transfer therapy. Adoptive T cell transfer therapy is a technique that can boost the natural immune system's ability to combat cancer by enriching for and/or designing T cells that are able to effectively recognize, bind, and kill a diseased cell. CTLs can recognize and bind cancerous cells using T-cell receptors (TCR). TCRs contain a highly variable binding region that allow them to recognize a large range of antigens. TCRs bind to the major histocompatibility complex I (MHC I) of cancerous cells presenting an appropriate antigen. TCRs binding is highly specific, so only a small number of CTLs will be able to recognize a particular antigen. Once an antigen is recognized by CTLs binding to the MHC I complex of the cancerous cell, they activate to induce cellular death. Activated CTLs proliferate to fight the detected cancer.

CTLs administered in this therapy may be derived from the subject or may be derived from other biological sources. Methods for producing CTLs directed to a particular antigen are well known in the art, and can be harvested from an individual possessing a CTL directed to a particular antigen or produced outside of the body (ex vivo). For example, when treating cancer, cytotoxic T cells from a subject's tumor are isolated, the cytotoxic T cells with the greatest antitumor activity are identified, the identified cytotoxic T cells are cultured to produce large amounts of the most effective cells, and the cultured cytotoxic T cells are reintroduced into the subject to treat the cancer. CTLs can also be produced in healthy individuals using ex vivo techniques described in U.S. Pat. No. 5,962,318 and US2009/0324539, the contents of which are herein incorporated by reference for all purposes. The ex vivo methods described herein can be useful for individuals both before cancer onset or after cancer onset.

CAR-T cells are modified T cells which have been engineered to possess a cellular specificity domain that has not been produced naturally. The natural specificity domain of T cells are T-cell receptors that recognize a particular antigen presented on MHC class I molecules. In some embodiments, CAR-T cells possess a T-cell receptor that has not been naturally produced in a subject's body. In some embodiments, the cellular specificity domain is a monoclonal antibody that is specific for the targeted cells or tissue. CAR-T cells can be produced using any means known in the art. In some embodiments, cytotoxic T cells are harvested from a subject's blood, the cytotoxic T cells are genetically modified by inserting a gene that encodes for a receptor that recognizes an antigen specific to the cancer affecting the subject, the CAR-T cells are cultured and can be stored for later use or reintroduced into the subject's body to treat the cancer. For more information on the details of producing CAR-T cells, see, e.g. U.S. Pat. Nos. 9,102,760, 8,399,645, 8,975,071 and 8,916,381, the contents of which are herein incorporated by reference for all purposes.

In some embodiments, the present invention provides a composition comprising one or more compounds according to Formula (I) in combination with one or more antigen-specific immunotherapeutic agents and its application in a method of treating a cancer in a subject in need thereof. In certain instances, the composition further comprises a pharmaceutically acceptable excipient or diluent. In other instances, the composition is formulated for oral or parenteral administration.

In other embodiments, the present invention provides a method for treating cancer in a subject comprising administering to the subject an effective amount of one or more compounds according to Formula (I) in combination with one or more antigen-specific immunotherapeutic agents. In certain instances, the effective amount of one or more compounds according to Formula (I) is an amount sufficient to sensitize a cancer that is resistant to treatment with antigen-specific immunotherapeutic agents to such treatment. The compounds according to Formula (I) and antigen-specific immunotherapeutic agents can be delivered to a subject via the same route of administration (e.g. orally or parenterally) or via different routes of administration.

The following non-limiting examples of anticancer drugs in clinical trials for the treatment of CRPC can be combined with one or more compounds according to Formula (I):
Kinases: Masiviera masitinib, ipatasertib, dactolisib, BA3011, NUV-422, SRA737, VAL201, KX2-391, onvansertib (PCM-075), TL-118 and elimusertib (BAY 1895344);
Hormone signaling: Proxalutamide, AST-106, xentuzumab, seviteronel, galeterone, EPI-7386, FT-7051, PTVG-AR (MVI-118), prostreat and SXLO1;
Prostate-specific antigens: ¹⁷⁷Lu-PSMA-617, HPN424, Advaxis PSA vaccine, ATL101, EC1169, P-PSMA-101 and APV0442;
Cell signaling: Bavituximab, Trodelvy sacituzumab govitecan, DSTP3086S and Veyonda (NOX66); Immune mediator: Yervoy (ipilimumab), Keytruda pembrolizumab, Prostvac (rilimogene galvacirepvec/rilimogene glafolivec), ONO-4538 and Korlym mifepristone;
Angiogenesis: Cometriq, avastin bevacizumab and recentin cediranib;
DNA damage response (PARP inhibitor): Olaparib, rucaparib, niraparib, talazoparib, veliparib, pamiparib, iniparib, CEP 9722 and E7016
Epigenetics: CPI-1205, ZEN-3694 and ZEN-3694;
Metabolism: Etruma etrumadenant, indoximod and EOS100850 (EOS-850);
Protein degradation: ARV-110, CC-94676 and KPG-818;
Transcriptional regulator: Orphagen's antagonists to SF1;
Osteoclast inhibitors e.g. denosumab and zoledronic acid.

### Diseases and Conditions

In certain aspects, a cancer can be treated or prevented by administering one or more compounds according to Formula (I). In some embodiments, the one or more compounds according to Formula (I) are administered in combination with an anticancer drug. Cancer generally includes any of various malignant neoplasms characterized by the proliferation of anaplastic cells that tend to invade surrounding tissue and metastasize to new body sites. Non-limiting examples of different types of cancer suitable for treatment using the compositions of the present invention include prostate cancer, lung cancer, ovarian cancer, breast cancer, bladder cancer, thyroid cancer, liver cancer, pleural cancer, pancreatic cancer, cervical cancer, testicular cancer, colon cancer, anal cancer, bile duct cancer, gastrointestinal carcinoid tumors, esophageal cancer, gall bladder cancer, rectal cancer, appendix cancer, small intestine cancer, stomach (gastric) cancer, renal cancer (i.e. renal cell carcinoma), cancer of the central nervous system, skin cancer, choriocarcinomas, head and neck cancers, bone cancer, osteogenic sarcomas, fibrosarcoma, neuroblastoma, glioma, endometrial cancer, melanoma, leukemia (e.g. acute lymphocytic leukemia, chronic lymphocytic leukemia, acute myelogenous leukemia, chronic myelogenous leukemia, or hairy cell leukemia), lymphoma (e.g. non-Hodgkin's lymphoma, Hodgkin's lymphoma, B-cell lymphoma, or Burkitt's lymphoma) and multiple myeloma.

In particular embodiments, the cancer is an epithelial cancer (e.g. prostate cancer, ovarian cancer, breast cancer and the like) or a blood cancer (e.g. leukemia, lymphoma, multiple myeloma). In some embodiments, the cancer is a prostate cancer. In certain embodiments, the prostate cancer is an advanced stage prostate cancer selected from one or more of metastatic prostate cancer, drug-resistant prostate cancer (e.g. anti-androgen-resistant prostate cancer such as enzalutamide-resistant prostate cancer, abiraterone-resistant prostate cancer, bicalutamide-resistant prostate cancer etc.; taxane-resistant prostate cancer; docetaxel-resistant prostate cancer; and the like), hormone refractory prostate cancer, castration-resistant prostate cancer (CRPC), metastatic castration-resistant prostate cancer, AR-V7-induced drug-resistant prostate cancer such as AR-V7-induced anti-androgen-resistant prostate cancer (e.g. AR-V7-induced enzalutamide-resistant prostate cancer), AKRIC3-induced drug-resistant prostate cancer such as AKRIC3-induced anti-androgen-resistant prostate cancer (e.g. AKRIC3-induced enzalutamide-resistant prostate cancer) and combinations thereof.

In other embodiments, the cancer is a lung cancer, breast cancer, liver cancer, ovarian cancer, endometrial cancer, bladder cancer, colon cancer, lymphoma or a glioma. In certain instances, the lung cancer is a non-small-cell lung cancer (NSCLC), K-Ras mutant lung cancer. BRAF mutant lung cancer, tyrosine kinase inhibitor-resistant lung cancer, small cell lung cancer (SCLC), adenocarcinoma (e.g. adenocarcinoma in situ), squamous cell carcinoma, large cell carcinoma, bronchial carcinoid or combinations thereof. In certain instances, the breast cancer is triple-negative breast cancer (TNBC), tamoxifen-resistant breast cancer, radiation-resistant breast cancer, ductal carcinoma in situ, invasive ductal carcinoma, HER2-positive breast cancer, ER-positive breast cancer, inflammatory breast cancer, metastatic breast cancer, medullary carcinoma, tubular carcinoma, mucinous carcinoma (colloid) or combinations thereof. In certain instances, the liver cancer is a hepatocellular carcinoma (HCC), cholangiocarcinoma (bile duct cancer), angiosarcoma, hepatoblastoma or combinations thereof. In certain instances, the glioma is an ependymoma, astrocytoma (e.g. glioblastoma multiforme), oligodendroglioma, brainstem glioma, optic nerve glioma or combinations thereof (e.g. mixed glioma).

In another aspect, the present invention relates to a compound of Formula (I) as disclosed in any of the preceding embodiments for use in a method of treating cancer in a subject in need thereof.

In another aspect, the present invention relates to use of a compound of Formula (I) as disclosed in any of the preceding embodiments for the manufacture of a medicament for treating cancer in a subject in need thereof.

In another aspect, the present invention relates to use of a compound of Formula (I) as disclosed in any of the preceding embodiments in a method for treating cancer in a subject in need thereof.

### Pharmaceutical Compositions

The pharmaceutical compositions of the present invention encompass compositions made by admixing one or more compounds according to Formula (I) and a pharmaceutically acceptable carrier and/or excipient or diluent. Such compositions are suitable for pharmaceutical use in an animal or human.

The pharmaceutical compositions of the present invention may be prepared by any of the methods well-known in the art of pharmacy. Pharmaceutically acceptable carriers suitable for use with the present invention include any of the standard pharmaceutical carriers, buffers and excipients, including phosphate-buffered saline solution, water and emulsions (such as an oil/water or water/oil emulsion) and various types of wetting agents and/or adjuvants. Suitable pharmaceutical carriers and their formulations are described in Remington's Pharmaceutical Sciences (Mack Publishing Co., Easton, 19th ed. 1995). Preferred pharmaceutical carriers depend upon the intended mode of administration of the active agent(s).

The pharmaceutical compositions of the present invention can include one or more compounds according to Formula (I), one or more anticancer drugs such as an anti-androgen drug (e.g. enzalutamide, abiraterone and/or bicalutamide) and/or a chemotherapeutic agent (e.g. tamoxifen and/or a taxane such as docetaxel) or any pharmaceutically acceptable salts thereof, as an active ingredient and a pharmaceutically acceptable carrier and/or excipient or diluent. In particular embodiments, the pharmaceutical composition can include one or more compounds according to Formula (I), and an anti-androgen drug, such as enzalutamide. A pharmaceutical composition may optionally contain other therapeutic ingredients.

The compounds of the present invention can be combined as the active ingredient in intimate admixture with a suitable pharmaceutical carrier and/or excipient according to conventional pharmaceutical compounding techniques. Any carrier and/or excipient suitable for the form of preparation desired for administration is contemplated for use with the compounds disclosed herein.

In some embodiments, the pharmaceutical compositions comprising one or more compounds according to Formula (I) and the pharmaceutical compositions comprising one or more anticancer drugs are prepared as a single medicament. In other embodiments, the pharmaceutical compositions comprising one or more compounds according to Formula (I) and the pharmaceutical compositions comprising one or more anticancer drugs are prepared as separate medicaments.

The pharmaceutical compositions of the present invention include formulations suitable for topical, parenteral, pulmonary, nasal, rectal or oral administration. The most suitable route of administration in any given case will depend in part on the nature and severity of the cancer condition and also optionally the stage of the cancer.

In embodiments where the compound according to Formula (I) is administered in combination with an anticancer drug, the administration of the compound according to Formula (I) and the anticancer drug may be administered using the same or a different administration route. For example, in some embodiments, both the compound according to Formula (I) and the anticancer drug may be administered orally or parenterally (e.g. intravenously). For example, in other embodiments, the compound according to Formula (I) may be administered orally, while the anticancer drug may be administered parenterally (e.g. intravenously) or vice versa.

Other preferred compositions include compositions suitable for systemic (enteral or parenteral) administration. Systemic administration includes oral, rectal, sublingual or sublabial administration. In some embodiments, the compositions may be administered via a syringe or intravenously.

Compositions for pulmonary administration include, but are not limited to, dry powder compositions consisting of the powder of a compound described herein, or a salt thereof, and the powder of a suitable carrier and/or lubricant. The compositions for pulmonary administration can be inhaled from any suitable dry powder inhaler device known to a person skilled in the art.

Compositions for systemic administration include, but are not limited to, dry powder compositions consisting of the composition as set forth herein and the powder of a suitable carrier and/or excipient. The compositions for systemic administration can be represented by, but not limited to, tablets, capsules, pills, syrups, solutions, and suspensions.

In some embodiments, the present invention provides compositions further including a pharmaceutical surfactant. In other embodiments, the present invention provides compositions further including a cryoprotectant. In some embodiments, the cryoprotectant is selected from the group consisting of glucose, sucrose, trehalose, lactose, sodium glutamate, PVP, HPβCD, CD, glycerol, maltose, mannitol and saccharose.

In some embodiments, the present invention provides a pharmaceutical composition including one or more compounds according to Formula (I) and a pharmaceutically acceptable excipient. In some embodiments, the present invention provides a pharmaceutical composition including one or more compounds according to Formula (I) and one or more anticancer drugs such as an anti-androgen drug (e.g. enzalutamide, abiraterone and/or bicalutamide) and/or a chemotherapeutic agent (e.g. tamoxifen and/or a taxane such as docetaxel), in combination with a pharmaceutically acceptable excipient. In particular embodiments, the present invention provides a pharmaceutical composition including one or more compounds according to Formula (I) and an anti-androgen drug, such as enzalutamide, in combination with a pharmaceutically acceptable excipient. In some of these embodiments, the pharmaceutically acceptable excipient includes a salt or a diluent.

In some embodiments, the present invention provides compositions including an effective amount of one or more compounds according to Formula (I). In some embodiments, the composition is formulated for oral administration or parenteral (e.g. intravenous) administration and includes one or more compounds according to Formula (I) and at least one member selected from the group consisting of an aqueous solution and a buffer solution. In some embodiments, the composition can include an effective amount of one or more compounds according to Formula (I) and one or more anticancer drugs such as an anti-androgen drug (e.g. enzalutamide, abiraterone and/or bicalutamide) and/or a chemotherapeutic agent (e.g. tamoxifen and/or a taxane such as docetaxel).

Pharmaceutical compositions or medicaments for use in the present invention can be formulated by standard techniques using one or more physiologically acceptable carriers or excipients. Suitable pharmaceutical carriers are described herein and in Remington: The Science and Practice of Pharmacy, 21^{st} Ed., University of the Sciences in Philadelphia, Lippencott Williams & Wilkins (2005).

For oral administration, a pharmaceutical composition or a medicament can take the form of, e.g. a tablet or a capsule prepared by conventional means with a pharmaceutically acceptable excipient. Preferred are tablets and gelatin capsules comprising the active ingredient(s), together with (a) diluents or fillers, e.g. lactose, dextrose, sucrose, mannitol, sorbitol, cellulose (e.g. ethyl cellulose, microcrystalline cellulose), glycine, pectin, polyacrylates and/or calcium hydrogen phosphate, calcium sulfate, (b) lubricants, e.g. silica, anhydrous colloidal silica, talcum, stearic acid, its magnesium or calcium salt (e.g. magnesium stearate or calcium stearate), metallic stearates, colloidal silicon dioxide, hydrogenated vegetable oil, corn starch, sodium benzoate, sodium acetate and/or polyethyleneglycol; for tablets also (c) binders, e.g. magnesium aluminum silicate, starch paste, gelatin, tragacanth, methylcellulose, sodium carboxymethylcellulose, polyvinylpyrrolidone and/or hydroxypropyl methylcellulose; if desired (d) disintegrants, e.g. starches (e.g. potato starch or sodium starch), glycolate, agar, alginic acid or its sodium salt, or effervescent mixtures; (e) wetting agents, e.g. sodium lauryl sulfate and/or (f) absorbents, colorants, flavors and sweeteners. In some embodiments, the tablet contains a mixture of hydroxypropyl methylcellulose, polyethyleneglycol 6000 and titanium dioxide. Tablets may be either film coated or enteric coated according to methods known in the art.

Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring and/or sweetening agents as appropriate. If desired, preparations for oral administration can be suitably formulated to give controlled release of the active compound.

Controlled release parenteral formulations of the compositions of the present invention can be made as implants, oily injections, or as particulate systems. For abroad overview of delivery systems see, Banga, A. J., Therapeutic Peptides and Proteins: Formulation, Processing, and Delivery Systems, Technomic Publishing Company, Inc., Lancaster, Pa. (1995) incorporated herein by reference. Particulate systems include microspheres, microparticles, microcapsules, nanocapsules, nanospheres and nanoparticles. Polymers can be used for ion-controlled release of compositions of the present invention. Various degradable and nondegradable polymeric matrices for use in controlled drug delivery are known in the art (Acc. Chem. Res. 1993; 26:537). For example, the block copolymer, polaxamer 407 exists as a viscous yet mobile liquid at low temperatures but forms a semisolid gel at body temperature. It has shown to be an effective vehicle for formulation and sustained delivery of recombinant interleukin 2 and urease (Pharm. Res. 1992; 9:425; J. Parenter. Sci. Technol. 1990; 44:58). Alternatively, hydroxyapatite has been used as a microcarrier for controlled release of proteins (Int. J. Pharm. 1994; 112:215). In yet another aspect, liposomes are used for controlled release as well as drug targeting of the lipid-capsulated drug (Betageri et al., Liposome Drug Delivery Systems, Technomic Publishing Co., Inc., Lancaster, Pa. (1993)). Numerous additional systems for controlled delivery of therapeutic proteins are known. See, e.g., U.S. Pat. Nos. 5,055,303, 5,188,837, 4,235,871, 4,501,728, 4,837,028, 4,957,735, 5,019,369, 5,055,303, 5,514,670, 5,413,797, 5,268,164, 5,004,697, 4,902,505, 5,506,206, 5,271,961, 5,254,342 and 5,534,496, each of which is incorporated herein by reference.

In another aspect, the present invention relates to a pharmaceutical composition as disclosed in any of the preceding embodiments for use in a method of treating cancer in a subject in need thereof.

In another aspect, the present invention relates to use of a pharmaceutical composition as disclosed in any of the preceding embodiments for the manufacture of a medicament for treating cancer in a subject in need thereof.

In another aspect, the present invention relates to use of a pharmaceutical composition as disclosed in any of the preceding embodiments in a method for treating cancer in a subject in need thereof.

### Methods of Administration

Pharmaceutical compositions or medicaments comprising one or more compounds according to Formula (I) can be administered to a subject at a therapeutically effective dose to treat the subject's cancer, as described herein. In some embodiments, pharmaceutical compositions or medicaments comprising one or more compounds according to Formula (I) can be co-administered to a subject in combination with an effective amount of an anticancer drug at a therapeutically effective dose to treat the subject's cancer, as described herein. In some embodiments, the pharmaceutical composition or medicament comprising one or more compounds according to Formula (I) is administered to a subject in an amount sufficient in to elicit an effective therapeutic response in the subject. In some embodiments, the pharmaceutical composition or medicament comprising one or more compounds according to Formula (I) can be co-administered to a subject at a therapeutically effective dose in combination with an effective amount of an anticancer drug to elicit an effective therapeutic response in the subject.

In certain methods of treating cancer, set forth herein, the methods comprise first administering one or more compounds according to Formula (I) to a patient having cancer and then administering an anticancer drug, such as an anti-androgen drug and/or a chemotherapeutic agent, to the patient. In certain methods of treating cancer, set forth herein, the methods comprise first administering an anticancer drug, such as an anti-androgen drug and/or a chemotherapeutic agent, to a patient having cancer, and then administering one or more compounds according to Formula (I) to the patient. In certain methods of treating cancer, set forth herein, the methods comprise co-administering one or more compounds according to Formula (I) with an anticancer drug, such as an anti-androgen drug and/or a chemotherapeutic agent, to a patient having cancer.

In some embodiments, the methods of administration comprise administering one or more compounds according to Formula (I) alone or in combination with enzalutamide to a patient in need thereof. In other embodiments, the methods of administration comprise administering one or more compounds according to Formula (I) alone or in combination with abiraterone to a patient in need thereof. In yet other embodiments, the methods comprise administering one or more compounds according to Formula (I) alone or in combination with bicalutamide to a patient in need thereof. In still yet other embodiments, the methods comprise administering one or more compounds according to Formula (I) alone or in combination with a taxane such as docetaxel to a patient in need thereof. In further embodiments, the methods comprise administering one or more compounds according to Formula (I) alone or in combination with tamoxifen to a patient in need thereof.

In certain embodiments, the present invention provides a method of delivering an effective amount of one or compounds according to Formula (I) to a patient having cancer such as prostate cancer (e.g. CRPC).

The compound according to Formula (I) described herein are useful in the manufacture of a pharmaceutical composition or a medicament. A pharmaceutical composition or medicament can be administered to a subject in need thereof, e.g. a patient having a cancer such as prostate cancer (e.g. CRPC), lung cancer, breast cancer, liver cancer, ovarian cancer, endometrial cancer, bladder cancer, colon cancer, lymphoma or a glioma.

Pharmaceutical compositions or medicaments for use in the present invention can be formulated by standard techniques using one or more physiologically acceptable carriers or excipients. Suitable pharmaceutical carriers are described herein and in “Remington's Pharmaceutical Sciences” by E. W. Martin. Compounds and agents of the present invention and their physiologically acceptable salts and solvates can be formulated for administration by any suitable route, including via inhalation, topically, nasally, orally, intravenously, parenterally or rectally.

### 1. Routes of Administration

Typical formulations for topical administration include creams, ointments, sprays, lotions and patches. The pharmaceutical composition can, however, be formulated for any type of administration, e.g. intradermal, subdermal, intravenous, intramuscular, intranasal, intracerebral, intratracheal, intraarterial, intraperitoneal, intravesical, intrapleural, intracoronary or intratumoral injection, with a syringe or other devices. Formulation for administration by inhalation (e.g. aerosol) or for oral or rectal administration is also contemplated.

Suitable formulations for transdermal application include an effective amount of one or more compounds described herein, optionally with a carrier. Preferred carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used.

For oral administration, a pharmaceutical composition or a medicament can take the form of, for example, a tablet or a capsule prepared by conventional means with a pharmaceutically acceptable excipient. The present invention provides tablets and gelatin capsules comprising one or more compounds according to Formula (I) alone or in combination with other compounds such as anticancer drugs, or a dried solid powder of these drugs, together with (a) diluents or fillers, e.g. lactose, dextrose, sucrose, mannitol, sorbitol, cellulose (e.g. ethyl cellulose, microcrystalline cellulose), glycine, pectin, polyacrylates and/or calcium hydrogen phosphate, calcium sulfate, (b) lubricants, e.g. silica, talcum, stearic acid, magnesium or calcium salt, metallic stearates, colloidal silicon dioxide, hydrogenated vegetable oil, corn starch, sodium benzoate, sodium acetate and/or polyethyleneglycol; for tablets also (c) binders, e.g. magnesium aluminum silicate, starch paste, gelatin, tragacanth, methylcellulose, sodium carboxymethylcellulose, polyvinylpyrrolidone and/or hydroxypropyl methylcellulose; if desired (d) disintegrants, e.g. starches (e.g. potato starch or sodium starch), glycolate, agar, alginic acid or its sodium salt, or effervescent mixtures; (e) wetting agents, e.g. sodium lauryl sulphate and/or (f) absorbents, colorants, flavors and sweeteners.

Tablets may be either film coated or enteric coated according to methods known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives or hydrogenated edible fats; emulsifying agents, for example, lecithin or acacia; non-aqueous vehicles, for example, almond oil, oily esters, ethyl alcohol or fractionated vegetable oils; and preservatives, for example, methyl or propyl-p-hydroxybenzoates or sorbic acid. The preparations can also contain buffer salts, flavoring, coloring and/or sweetening agents as appropriate. If desired, preparations for oral administration can be suitably formulated to give controlled release of the active compound(s).

The compositions and formulations set forth herein can be formulated for parenteral administration by injection, for example by bolus injection or continuous infusion. Formulations for injection can be presented in unit dosage form, for example, in ampoules or in multi-dose containers, with an added preservative. Injectable compositions are preferably aqueous isotonic solutions or suspensions, and suppositories are preferably prepared from fatty emulsions or suspensions. The compositions may be sterilized and/or contain adjuvants, such as preserving, stabilizing, wetting or emulsifying agents, solution promoters, salts for regulating the osmotic pressure and/or buffers. Alternatively, the active ingredient(s) can be in powder form for constitution with a suitable vehicle, for example, sterile pyrogen-free water, before use. In addition, they may also contain other therapeutically valuable substances. The compositions are prepared according to conventional mixing, granulating or coating methods, respectively, and contain about 0.1 to 75%, preferably about 1 to 50%, of the active ingredient(s).

For administration by inhalation, the compositions of the present invention may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound(s) and a suitable powder base, for example, lactose or starch.

The compositions set forth herein can also be formulated in rectal compositions, for example, suppositories or retention enemas, for example, containing conventional suppository bases, for example, cocoa butter or other glycerides.

Furthermore, the active ingredient(s) can be formulated as a depot preparation. Such long-acting formulations can be administered by implantation (for example, subcutaneously or intramuscularly) or by intramuscular injection. Thus, for example, one or more of the compounds described herein can be formulated with suitable polymeric or hydrophobic materials (for example as an emulsion in an acceptable oil) or ion exchange resins, or as sparingly soluble derivatives, for example, as a sparingly soluble salt.

In particular embodiments, a pharmaceutical composition or medicament of the present invention can comprise (i) an effective amount of one or more compounds according to Formula (I) and (ii) optionally an anticancer drug such as an anti-androgen drug (e.g. enzalutamide, abiraterone, bicalutamide), a chemotherapeutic agent such as a taxane (e.g. docetaxel) or tamoxifen, and combinations thereof. The therapeutic agent(s) may be used individually, sequentially, or in combination with one or more other such therapeutic agents (e.g. a first therapeutic agent, a second therapeutic agent, a compound of the present invention, etc.). Administration may be by the same or different route of administration or together in the same pharmaceutical formulation.

### 2. Dosage

Pharmaceutical compositions or medicaments can be administered to a subject at a therapeutically effective dose to prevent, treat, sensitize, or control a cancer such as prostate cancer (e.g. CRPC), lung cancer, breast cancer, liver cancer, ovarian cancer, endometrial cancer, bladder cancer, colon cancer, lymphoma or a glioma as described herein. The pharmaceutical composition or medicament is administered to a subject in an amount sufficient to elicit an effective therapeutic response in the subject. An effective therapeutic response includes a response that at least partially arrests or slows the symptoms or complications of the cancer. An amount adequate to accomplish this is defined as a “therapeutically effective dose.”

The dosage of active agents administered is dependent on the subject's body weight, age, individual condition, surface area or volume of the area to be treated and on the form of administration. The size of the dose also will be determined by the existence, nature and extent of any adverse effects that accompany the administration of a particular formulation in a particular subject. A unit dosage for oral administration to a mammal of about 50 to about 70 kg may contain between about 5 and about 500 mg, about 25 and about 200 mg, about 100 and about 1000 mg, about 200 and about 2000 mg, about 500 and about 5000 mg, or between about 1000 and about 2000 mg of the active ingredient. A unit dosage for oral administration to a mammal of about 50 to about 70 kg may contain about 10 mg, 20 mg, 25 mg, 50 mg, 75 mg, 100 mg, 150 mg, 200 mg, 300 mg, 400 mg, 450 mg, 500 mg, 600 mg, 700 mg, 750 mg, 800 mg, 900 mg, 1000 mg, 1250 mg, 1500 mg or more of the active ingredient. Typically, a dosage of the active compound(s) of the present invention is a dosage that is sufficient to achieve the desired effect. Optimal dosing schedules can be calculated from measurements of active agent accumulation in the body of a subject. In general, dosage may be given once or more of daily, weekly or monthly. Persons of ordinary skill in the art can easily determine optimum dosages, dosing methodologies and repetition rates.

Optimum dosages, toxicity, and therapeutic efficacy of the compositions of the present invention may vary depending on the relative potency of the administered composition and can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀. Agents that exhibit large therapeutic indices are preferred. While agents that exhibit toxic side effects can be used, care should be taken to design a delivery system that targets such agents to the site of affected tissue to minimize potential damage to normal cells and, thereby, reduce side effects.

Optimal dosing schedules can be calculated from measurements of active ingredient accumulation in the body of a subject. In general, dosage is from about 1 ng to about 100 mg per kg of body weight and may be given once or more daily, weekly, monthly or yearly. Persons of ordinary skill in the art can easily determine optimum dosages, dosing methodologies and repetition rates. One of skill in the art will be able to determine optimal dosing for administration of one or more compounds according to Formula (I) to a human being following established protocols known in the art and the disclosure herein. The data obtained from, for example, animal studies (e.g. rodents and monkeys) can be used to formulate a dosage range for use in humans. The dosage of compounds of the present invention lies preferably within a range of circulating concentrations that include the ED₅s with little or no toxicity. The dosage can vary within this range depending upon the dosage form employed and the route of administration. For any composition for use in the methods of the invention, the therapeutically effective dose can be estimated initially from cell culture assays. A dose can be formulated in animal models to achieve a circulating plasma concentration range that includes the IC₅₀ (the concentration of the test compound that achieves a half-maximal inhibition of symptoms) as determined in cell culture. Such information can be used to more accurately determine useful doses in humans. Levels in plasma can be measured, for example, by high performance liquid chromatography (HPLC). In general, the dose equivalent of a chimeric protein, preferably a composition is from about 1 ng/kg to about 100 mg/kg for a typical subject.

A typical composition of the present invention for oral or intravenous administration can be about 25 to about 200 mg per patient per day; about 50 to about 500 mg per patient per day; about 100 to about 1000 mg per patient per day; or about 1000 to about 2000 mg per patient per day. Exemplary dosages include, but are not limited to, about 10 mg, 20 mg, 25 mg, 50 mg, 75 mg, 100 mg, 150 mg, 200 mg, 300 mg, 400 mg, 450 mg, 500 mg, 600 mg, 700 mg, 750 mg, 800 mg, 900 mg, 1000 mg, 1100 mg, 1200 mg or more of the active ingredient per patient per day. Methods for preparing administrable compositions will be known or apparent to those skilled in the art and are described in more detail in such publications as Remington: The Science and Practice of Pharmacy, 21′ Ed., University of the Sciences in Philadelphia, Lippencott Williams & Wilkins (2005).

Exemplary doses of the compositions described herein include milligram or microgram amounts of the composition per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a composition depend upon the potency of the composition with respect to the desired effect to be achieved. When one or more of these compositions is to be administered to a mammal, a physician, veterinarian, or researcher may, for example, prescribe a relatively low dose at first, subsequently increasing the dose until an appropriate response is obtained. In addition, it is understood that the specific dose level for any particular mammal subject will depend upon a variety of factors including the activity of the specific composition employed, the age, body weight, general health, gender, and diet of the subject, the time of administration, the route of administration, the rate of excretion, any drug combination, and the degree of expression or activity to be modulated.

In some embodiments, a pharmaceutical composition or medicament of the present invention is administered, e.g. in a daily dose in the range from about 0.5 mg of compound per kg of subject weight (0.5 mg/kg) to about 100 mg/kg. In another embodiment, the dose is a dose in the range of about 1 mg/kg to about 25 mg/kg. In yet another embodiment, the dose is about 2 mg/kg to about 10 mg/kg. A preferred dose is about 2, 3, 4, 5, 6, 7, 8, 9, 10, 12 or 15 mg/kg. The daily dose can be administered once per day or divided into subdoses and administered in multiple doses, e.g., twice, three times or four times per day. However, as will be appreciated by a skilled artisan, compositions described herein may be administered in different amounts and at different times. The skilled artisan will also appreciate that certain factors may influence the dosage and timing required to effectively treat a subject, including but not limited to the severity of the disease or malignant condition, previous treatments, the general health and/or age of the subject, and other diseases present. Moreover, treatment of a subject with a therapeutically effective amount of a composition can include a single treatment or, preferably, can include a series of treatments.

To achieve the desired therapeutic effect, compounds or agents described herein may be administered for multiple days at the therapeutically effective daily dose. Thus, therapeutically effective administration of compounds to treat prostate cancer in a subject may require periodic (e.g. daily) administration that continues for a period ranging from three days to two weeks or longer. Compositions set forth herein may be administered for at least three consecutive days, often for at least five consecutive days, more often for at least ten, and sometimes for 20, 30, 40 or more consecutive days. While consecutive daily doses are a preferred route to achieve a therapeutically effective dose, a therapeutically beneficial effect can be achieved even if the agents are not administered daily, so long as the administration is repeated frequently enough to maintain a therapeutically effective concentration of the agents in the subject. For example, one can administer the agents every other day, every third day, or, if higher dose ranges are employed and tolerated by the subject, once a week.

In some embodiments, the one or more compounds according to Formula (I) are orally administered. In some embodiments, the one or more compound according to Formula (I) are orally administered to a subject (e.g. an adult human) at a daily dose of approximately 150, 300, 450, 600, 750, 900, 1050, 1200 or more mg per day. In some embodiments, the one or more compounds according to Formula (I) are orally administered to a subject (e.g. an adult human) at a daily dose of between 100 and 1500 mg per day. In some embodiments, the one or more compounds according to Formula (I) are orally administered to a subject (e.g. an adult human) at a daily dose of approximately 10, 15, 20, 25, 30, 35, 40, 50, 60, 70, 80, 90, 100, 125, 150, 175, 200, 225, 250, 300, 350, 400, 500, 600, 700, 800, 900, 1000, 1100, 1200 or more mg per day. In some embodiments, the one or more compounds according to Formula (I) and an anticancer drug are orally co-administered. For example, the one or more compounds according to Formula (I) can be co-administered at a daily oral dose of between 25 and 1000 mg per day with the anticancer drug at a daily oral dose of between 25 and 2000 mg per day.

In some embodiments, the methods comprise sequentially administering one or more compounds according to Formula (I) followed by one or more anticancer drugs such as an anti-androgen drug (e.g. enzalutamide, abiraterone, bicalutamide), a chemotherapeutic agent such as a taxane (e.g. docetaxel) or tamoxifen and combinations thereof. In some embodiments, the methods comprise sequentially administering one or more anticancer drugs followed by one or more compounds according to Formula (I).

Following successful treatment, it may be desirable to have the subject undergo maintenance therapy to prevent the recurrence of the cancer.

Determination of an effective amount is well within the capability of those skilled in the art, especially in light of the detailed disclosure provided herein. Generally, an efficacious or effective amount of a composition is determined by first administering a low dose or small amount of the composition and then incrementally increasing the administered dose or dosages, adding a second or third medication as needed, until a desired effect of is observed in the treated subject with minimal or no toxic side-effects. Single or multiple administrations of the compositions are administered depending on the dosage and frequency as required and tolerated by the patient. In any event, the composition should provide a sufficient quantity of the compositions of this invention to effectively treat the patient. Generally, the dose is sufficient to treat or ameliorate symptoms or signs of disease without producing unacceptable toxicity to the patient.

### Kits, Containers, Devices and Systems

A wide variety of kits and systems can be prepared according to the present invention, depending upon the intended user of the kit and system and the particular needs of the user. In some embodiments, the present invention provides a kit that includes one or more compounds according to Formula (I). In other aspects, the present invention provides a kit that includes one or more compounds according to Formula (I) and one or more anticancer drugs such as an anti-androgen drug (e.g. enzalutamide, abiraterone and/or bicalutamide) and/or a chemotherapeutic agent (e.g. tamoxifen and/or a taxane such as docetaxel).

Some of the kits described herein can include a label describing a method of administering one or more compounds according to Formula (I) and/or one or more anticancer drugs. Some of the kits described herein can include a label describing a method of treating cancer in a subject with a cancer such as prostate cancer (e.g. CRPC), lung cancer, breast cancer, liver cancer, ovarian cancer, endometrial cancer, bladder cancer, colon cancer, lymphoma, or a glioma.

The compositions of the present invention, including but not limited to, compositions comprising one or more compounds according to Formula (I) and optionally one or more anticancer drugs may, if desired, be presented in a bottle, jar, vial, ampoule, tube, or other container-closure system approved by the Food and Drug Administration (FDA) or other regulatory body, which may provide one or more dosages containing the compounds. The package or dispenser may also be accompanied by a notice associated with the container in a form prescribed by a governmental agency regulating the manufacture, use or sale of pharmaceuticals, the notice indicating approval by the agency. In certain aspects, the kit may include a formulation or composition as described herein, a container closure system including the formulation or a dosage unit form including the formulation, and a notice or instructions describing a method of use as described herein.

In some embodiments, the kit includes a container which is compartmentalized for holding the various elements of a formulation (e.g. the dry ingredients and the liquid ingredients) or composition, instructions for making the formulation or composition, and instructions for administering the formulation or composition for enhancing the immune response in a subject with a cancer.

In certain embodiments, the kit may include the pharmaceutical preparation(s) in dehydrated or dry form, with instructions for its rehydration (or reconstitution) and administration.

Kits with unit doses of the compounds described herein, e.g. in oral, rectal, transdermal or injectable doses (e.g. for intramuscular, intravenous or subcutaneous injection), are provided. In such kits, an informational package insert describing the use and attendant benefits of the composition for enhancing the immune response in a subject with a cancer such as prostate cancer (e.g. CRPC), lung cancer, breast cancer, liver cancer, ovarian cancer, endometrial cancer, bladder cancer, colon cancer, lymphoma or a glioma may be included in addition to the containers containing the unit doses.

Some embodiments of the present invention provide packages that include one or more compounds according to Formula (I) and optionally one or more anticancer drugs such as an anti-androgen drug (e.g. enzalutamide, abiraterone, and/or bicalutamide) and/or a chemotherapeutic agent (e.g. tamoxifen and/or a taxane such as docetaxel).

### Experimental Part

The compounds of the present invention can be prepared as outlined in WO2012/101261, WO2012/101263 and WO2019/048541. An exemplary example from these patent applications is described as Example 1 below.

The present invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### Abbreviations

DMF N,N-dimethylformamide
DMSO dimethylsulfoxide
EA ethyl acetate
ESI electrospray ionization
FCC flash chromatography on silica gel
LCMS liquid chromatography-mass spectrometry
Ms methanesulfonyl
PE petroleum ether
rt room temperature (20±4° C.)
sat. saturated
THF tetrahydrofuran
TMS trimethylsilyl
Ts para-toluenesulfonyl

### Experimental Section

### Example 1

### Step 1: Methyl 3-(2-chloro-6-fluorophenyl)-5-methylisoxazole-4-carboxylate (1a)

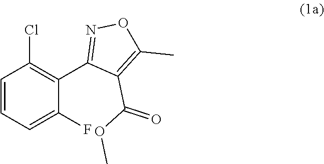 [SMILES: COC(=O)c1c(-c2c(F)cccc2Cl)noc1C]

To a stirred solution of (Z)-2-chloro-6-fluoro-N-hydroxybenzimidoyl chloride (117 g, 0.56 mol) and NaOEt in THF (600 mL) was added methyl 3-oxobutanoate (78 g, 0.67 mol). The mixture was stirred for 8 h at rt. The solvent was evaporated under reduced pressure and the residue was purified by FCC (PE:EA=2:1) to afford compound 1a as a colorless solid.

### Step 2: Methyl (E)-3-(2-chloro-6-fluorophenyl)-5-(2-(dimethylamino)vinyl)isoxazole-4-carboxylate (1b)

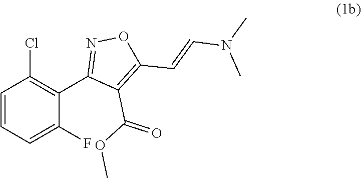 [SMILES: COC(=O)c1c(-c2c(F)cccc2Cl)noc1/C=C/N(C)C]

To a solution of compound 1a (90 g, 0.33 mol) in dry toluene (600 mL) was added 1-tert-butoxy-N,N,N′,N′-tetramethyhnethanediamine (134 mL, 0.69 mol). The mixture was heated under reflux for 6 h, cooled to rt, concentrated and dried in vacuum. PE was added to the oily residue and the crystalline product was collected by filtration to afford compound 1b as a solid.

### Step 3: Methyl (E)-3-(2-chloro-6-fluorophenyl)-5-(2-(dimethylamino)-4,4,4-trifluoro-3-oxobut-1-en-1-yl)isoxazole-4-carboxylate (1c)

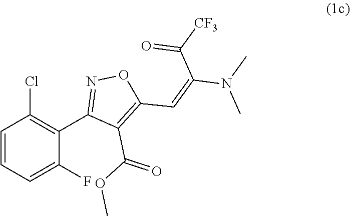 [SMILES: COC(=O)c1c(-c2c(F)cccc2Cl)noc1/C=C(\C(=O)C(F)(F)F)N(C)C]

To a solution of compound 1b (88 g, 0.27 mol) in dry CH₂Cl₂ (600 mL) was added dropwise trifluoroacetic anhydride (85 g, 0.40 mol) under ice-bath cooling. The mixture was stirred for 3 h at rt, concentrated and dried in vacuum. The oily residue crystallized with PE and the crystalline product was collected by filtration to afford compound 1c as a solid.

### Step 4: Methyl 3-(2-chloro-6-fluorophenyl)-5-(1-(3-chlorophenyl)-5-(trifluoromethyl)-1H-pyrazol-4-yl)isoxazole-4-carboxylate (1)

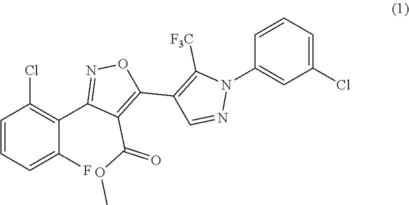 [SMILES: COC(=O)c1c(-c2c(F)cccc2Cl)noc1-c1cnn(-c2cccc(Cl)c2)c1C(F)(F)F]

To a solution of compound 1c (78 g, 0.19 mol) in dry ethanol was added 3-chlorophenylhydrazine (21.3 g, 0.15 mol) and N,N-diisopropylethylamine (19.3 g, 0.15 mol). The mixture was heated under reflux for 2 h, cooled to rt, evaporated and purified by FCC (PE:Et₂O=4:1) to afford compound 1 as a pale yellow solid. ¹H-NMR (400 MHz, DMSO-d6) δ 8.58 (s, 1H), 7.87 (s, 1H), 7.77-7.67 (m, 4H), 7.58 (d, J=8.0 Hz, 1H), 7.49 (dd, J=8.4, 9.2 Hz, 1H), 3.67 (s, 3H). LCMS (ESI): m/z 499.9/501.9 (M+H)⁺.

### Example 1/1

The following Examples were prepared as described in WO2012/101261 and WO2012/101263.

| # | structure |
|---|---|
| 1/1 | 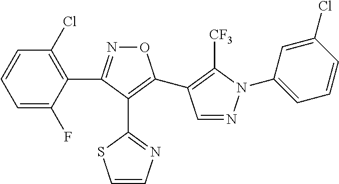 [SMILES: Fc1cccc(Cl)c1-c1noc(-c2cnn(-c3cccc(Cl)c3)c2C(F)(F)F)c1-c1nccs1] |

### Example 1/2

### Step 1: 3-Hydroxy-3-methylbutyl 4-methylbenzenesulfonate (1/2a)

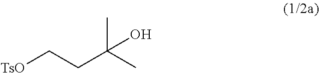 [SMILES: Cc1ccc(S(=O)(=O)OCCC(C)(C)O)cc1]

To a solution of 3-methylbutane-1,3-diol (10.0 g, 96.0 mmol) and Et₃N (20 mL) in CH₂Cl₂ (250 mL) were added p-toluenesulfonyl chloride (18.3 g). The mixture was stirred at rt overnight, the resulting suspension was filtered and the filter liquor was concentrated under vacuum. The residue was purified by FCC (PE:EA=2:1) to give compound 1/2a as a colorless oil.

### Step 2: 4-Hydrazineyl-2-methylbutan-2-ol (1/2b)

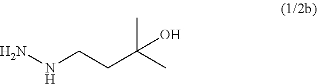

To a solution of compound 1/2a (23 g, 89 mmol) in ethanol (100 mL) was added hydrazine monohydrate (30 mL). The mixture was heated to 60° C. for 2 h and then concentrated to approx. 30 mL volume. Saturated aq. sodium hydroxide (40 mL) and THF (100 mL) were added and the organics collected, dried (Na₂SO₄), filtered and concentrated to afford compound 1/2b as a colorless oil, which was used without purification.

### Step 3: Ethyl 2-((dimethylamino)methylene)-4,4-difluoro-3-oxobutanoate (1/2c)

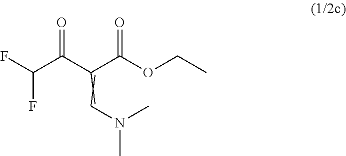

To a solution of ethyl 4,4-difluoro-3-oxobutanoate (10.0 g, 60.2 mmol) in toluene (20 mL) was added 1,1-dimethoxy-N,N-dimethylmethanamine (7.17 g, 60.2 mmol) and the mixture was stirred to rt for 16 h, concentrated under vacuum to afford compound 1/2c as a yellow oil which, was used for the next step without further purification. LCMS (ESI): m/z 222.1 (M+H)⁺.

### Step 4: Ethyl 5-(difluoromethyl)-1-(3-hydroxy-3-methylbutyl)-1H-pyrazole-4-carboxylate (1/2d)

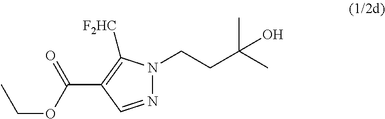 [SMILES: CCOC(=O)c1cnn(CCC(C)(C)O)c1C(F)F]

To a solution of compound 1/2c (12 g, 54 mmol) and compound 1/2b (7.0 g, 59 mmol) in EtOH (80 mL) was added Et₃N (22 mL). The solution was stirred at rt for 16 hour and extracted with EA (3×100 mL). The combined organic layer was washed with brine (100 mL), dried over Na₂SO₄, concentrated and purified by FCC (PE:EA=5:1) to give compound 1/2d as a colorless oil.

### Step 5: Ethyl 5-(difluoromethyl)-1-(3-methyl-3-((trimethylsilyl)oxy)butyl)-1H-pyrazole-4-carboxylate (1/2e)

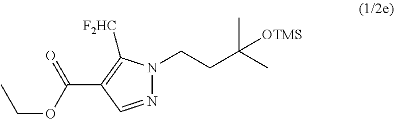

A solution of compound 1/2d (1.0 g, 3.6 mmol), in (Me₃Si)₂NH (10 mL) was added TMSCl (391 mg). The mixture was stirred at rt for 16 h, diluted with CH₂Cl₂ (50 mL), subsequently washed with water (20 mL) and saturated aq. NH₄Cl (20 mL). The organic layer was dried over Na₂SO₄, concentrated and purified by FCC (PE:EA=10:1) to give compound 1/2e as a colorless oil.

### Step 6: 1-(5-(Difluoromethyl)-1-(3-methyl-3-((trimethylsilyl)oxy)butyl)-1H-pyrazol-4-yl)-2-(pyrimidin-2-yl)ethan-1-one (1/21)

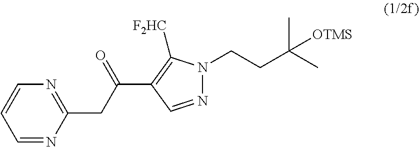

To a solution of compound 1/2e (1.0 g, 2.9 mmol) in THF (20 mL) was added lithium bis(trimethylsilyl)amide (1.6M in THF, 4.0 mL, 6.4 mmol) at −10° C. under a N₂ atmosphere. The mixture was stirred at this temperature for 30 min and 2-methylpyrimidine (0.33 g, 3.5 mmol) was added. The mixture was stirred at rt for 8 h, quenched with ice water (100 mL) and extracted with EA (3×80 mL). The combined organic layer was dried over Na₂SO₄, concentrated and purified FCC (PE:EA=1:1) to give compound 1/2f as a yellow oil.

### Step 7: 3-(2-Chloro-6-fluorophenyl)-5-(5-(difluoromethyl)-1-(3-methyl-3-((trimethylsilyl)oxy)butyl)-1H-pyrazol-4-yl)-4-(pyrimidin-2-yl)isoxazole (1/2g)

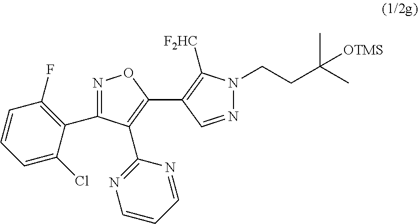

To a solution of compound 1/2f (0.30 g, 0.76 mmol) in dry DMF (10 ML) was added NaH (90 mg, 3.75 mmol) at 0° C. and the mixture was stirred at rt for 20 min. Then 2-chloro-6-fluoro-N-hydroxybenzimidoyl chloride (466 mg, 2.24 mmol) was added and the mixture was stirred at 60° C. for 3 h, cooled to rt, quenched with saturated aq. NH₄Cl and extracted with EA (3×80 mL). The combined organic layer was dried over Na₂SO₄, concentrated and purified by FCC (PE:EA=5:1) to give compound 1/2g as a colorless oil.

### Step 8: 4-(4-(3-(2-Chloro-6-fluorophenyl)-4-(pyrimidin-2-yl)isoxazol-5-yl)-5-(difluoromethyl)-1H-pyrazol-1-yl)-2-methylbutan-2-ol (1/2)

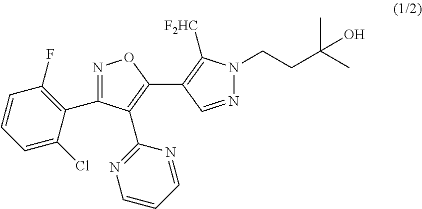 [SMILES: CC(C)(O)CCn1ncc(-c2onc(-c3c(F)cccc3Cl)c2-c2ncccn2)c1C(F)F]

To a solution of compound 1/2g (280 mg, 0.51 mmol) in MeOH (5 mL) was added 3M HCl/MeOH (1 mL) and the mixture was stirred at rt for 1 h, concentrated and purified by reversed-phase chromatography(C18) (MeCN: 0.05% NH₄HCO₃=10-100%) to give compound 1/2 as a white solid. ¹H-NMR (500 MHz, DMSO-d₆) δ: 8.67 (d, J=5.0 Hz, 2H), 8.21 (s, 1H), 7.63-7.32 (m, 5H), 4.56 (s, 1H), 4.45-4.41 (m, 2H), 1.98-1.94 (m, 2H), 1.18 (s, 6H). LCMS (ESI): m/z 478.2 (M+H)⁺.

### Example 2

### Step 1: (3-(2-Chloro-6-fluorophenyl)-5-(1-(3-chlorophenyl)-5-(trifluoromethyl)-1H-pyrazol-4-yl)isoxazol-4-yl)methanol (2a)

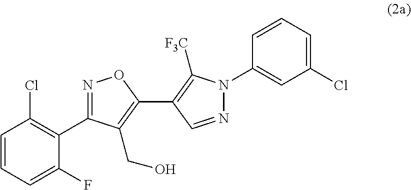 [SMILES: OCc1c(-c2c(F)cccc2Cl)noc1-c1cnn(-c2cccc(Cl)c2)c1C(F)(F)F]

To a solution of compound 1 (350 mg, 0.70 mmol) in MeOH (5 mL) was added NaBH₄ (54 mg, 1.40 mmol) at 0° C. and the mixture was stirred for 2 h at 0° C. Additional NaBH₄ (54 mg, 1.40 mmol) was added and the mixture was stirred at rt overnight, quenched with aq. NH₄Cl (20 mL) and extracted with EA (3×30 mL). The combined organic layer was washed with brine (30 mL), dried over Na₂SO₄, filtered, concentrated and purified by FCC (PE:EA=2.5:1) to afford compound 2a as a yellow oil. LCMS (ESI): m/z 471.9 (M+H)⁺.

### Step 2: (3-(2-Chloro-6-fluorophenyl)-5-(1-(3-chlorophenyl)-5-(trifluoromethyl)-1H-pyrazol-4-yl)isoxazol-4-yl)methyl methanesulfonate (2b)

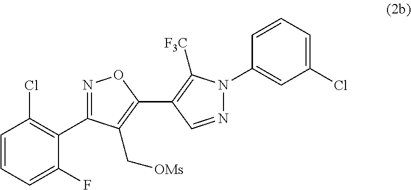 [SMILES: CS(=O)(=O)OCc1c(-c2c(F)cccc2Cl)noc1-c1cnn(-c2cccc(Cl)c2)c1C(F)(F)F]

To a solution of compound 2a (230 mg, 0.49 mmol) in CH₂Cl₂ (5 mL) was added Et₃N (99 mg, 0.98 mmol) and methanesulfonyl chloride (84 mg, 0.74 mmol) at 0° C. and the mixture was stirred for 1 h at rt, concentrated and purified by FCC (PE:EA=5:1) to afford compound 2b as a yellow oil. LCMS (ESI): m/z 550.0 (M+H)⁺.

### Step 3: 3-(2-Chloro-6-fluorophenyl)-5-(1-(3-chlorophenyl)-5-(trifluoromethyl)-1H-pyrazol-4-yl)-4-(cyclopropoxymethyl)isoxazole (2)

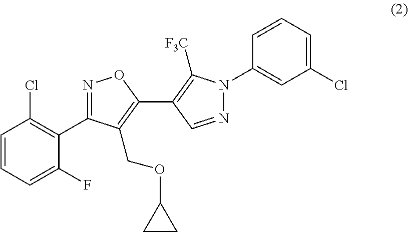 [SMILES: Fc1cccc(Cl)c1-c1noc(-c2cnn(-c3cccc(Cl)c3)c2C(F)(F)F)c1COC1CC1]

To a solution of compound 2b (110 mg, 0.20 mmol) in THF (3 mL) was added cyclopropanol (16 mg, 0.30 mmol) and t-BuOK (45 mg, 0.40 mmol) at rt. The mixture was stirred at rt overnight, quenched with aq. NH₄Cl (20 mL) and extracted with EA (3×30 mL). The combined organic layer was washed with brine (30 mL), dried over Na₂SO₄, filtered, concentrated and purified by prep-HPLC to afford compound 2 as a yellow oil. ¹H-NMR (400 MHz, DMSO-d₆) δ: 8.43 (s, 1H), 7.68 (s, 1H), 7.76-7.66 (m, 4H), 7.60 (d, J=8.0 Hz, 1H), 7.49 (t, J=8.6 Hz, 1HR), 4.36 (s, 2H), 3.19-3.10 (m, 1H), 0.29-0.24 (m, 2H), 0.13-0.11 (m, 2H). LCMS (ESI): m/z 511.7 (M+H)⁺.

### Example 2/1 to 2/10

The following Examples were prepared as described in WO2019/048541.

| # | structure |
|---|---|
| 2/1 | 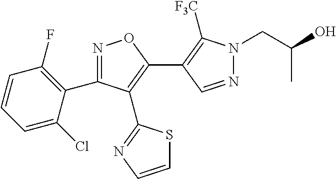 [SMILES: C[C@H](O)Cn1ncc(-c2onc(-c3c(F)cccc3Cl)c2-c2nccs2)c1C(F)(F)F] |
| 2/2 | 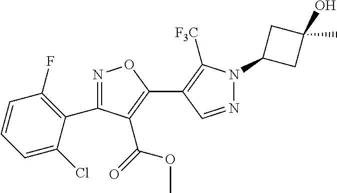 [SMILES: COC(=O)c1c(-c2c(F)cccc2Cl)noc1-c1cnn([C@H]2C[C@@](C)(O)C2)c1C(F)(F)F] |
| 2/3 | 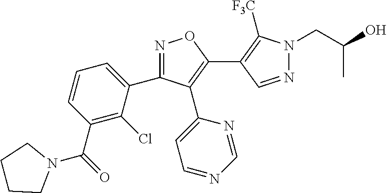 [SMILES: C[C@H](O)Cn1ncc(-c2onc(-c3cccc(C(=O)N4CCCC4)c3Cl)c2-c2ccncn2)c1C(F)(F)F] |
| 2/4 | 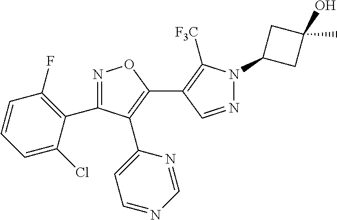 [SMILES: C[C@]1(O)C[C@@H](n2ncc(-c3onc(-c4c(F)cccc4Cl)c3-c3ccncn3)c2C(F)(F)F)C1] |
| 2/5 | 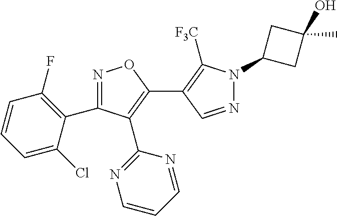 [SMILES: C[C@]1(O)C[C@@H](n2ncc(-c3onc(-c4c(F)cccc4Cl)c3-c3ncccn3)c2C(F)(F)F)C1] |
| 2/6 | 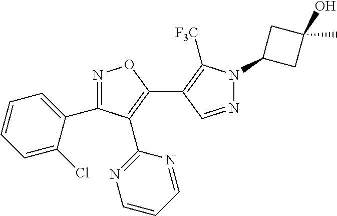 [SMILES: C[C@]1(O)C[C@@H](n2ncc(-c3onc(-c4ccccc4Cl)c3-c3ncccn3)c2C(F)(F)F)C1] |
| 2/7 | 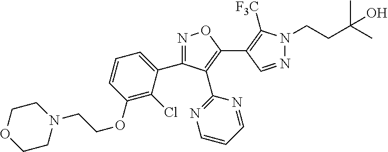 [SMILES: CC(C)(O)CCn1ncc(-c2onc(-c3cccc(OCCN4CCOCC4)c3Cl)c2-c2ncccn2)c1C(F)(F)F] |
| 2/8 | 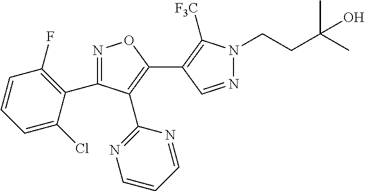 [SMILES: CC(C)(O)CCn1ncc(-c2onc(-c3c(F)cccc3Cl)c2-c2ncccn2)c1C(F)(F)F] |
| 2/9 | 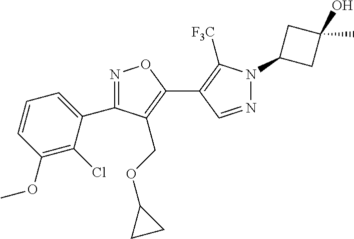 |
| 2/10 | 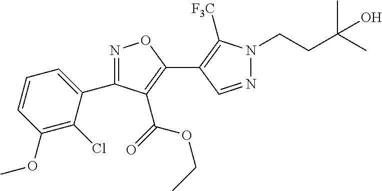 [SMILES: CCOC(=O)c1c(-c2cccc(OC)c2Cl)noc1-c1cnn(CCC(C)(C)O)c1C(F)(F)F] |

### Example 200: Cytokine Assay—Analysis of Proliferation of and Cytokine Production by Human PBMC Stimulated with PHA

Peripheral blood mononuclear cells (PBMC) from healthy human donors were purified using Accuspin™ System-Histopaque-1077 (Sigma) according to the protocol recommended by the manufacturer. Purified PBMC were then washed twice with phosphate-buffered saline (PBS) and resuspended in RPMI1640 culture medium supplemented with 10% dialyzed heat inactivated fetal calf serum, 1.5 mM L-glutamine, 100 U penicillin/mL and 100 mg streptomycin/mL (all from PAN Biotech, Aidenbach, Germany). For stimulation, PBMC were seeded at 1×10⁵ cells/well, activated with 2 gg/mL phytohaemagglutinin (PHA, Sigma) and incubated with the test compounds for 48 hours. IL-17A, IL-17F and INFγ were then determined in the culture supernatant using a Luminex BioPlex system, following the manufacturer's instructions (BioRad, Munich, Germany). To determine the IC₅₀, compounds were titrated semilogarithmically.

Cell proliferation was analyzed using the BrdU based cell proliferation ELISA from Roche (Mannheim, Germany) according to the manufacturer's instructions.

Cytokines were determined in the aforementioned culture supernatant using the following methods: IL-17A was measured using the human homodimer IL-17A ELISA Ready Set Go Kit from cBioscience (Frankfurt, Germany); IL-17F using the human IL-17F ELI-Pair from Holzel Diagnostica GmbH (K51n, Germany); and IFNγ using the OptEIA human IFN-g ELISA from BD Bioscience (Heidelberg, Germany), all following the manufacturer's instructions.

| Example # | IL-17A inhibition | IL-17F inhibition | IFNγ inhibition |
|---|---|---|---|
| 1 | +++ | +++ | +++ |
| 1/1 | +++ | +++ | +++ |
| 2/1 | +++ | +++ | +++ |
| 2/2 | +++ | +++ | +++ |
| 2/3 | ++ | ++ | ++ |
| 2/4 | ++ | +++ | +++ |
| 2/5 | +++ | +++ | +++ |
| 2/6 | +++ | +++ | +++ |
| 2/7 | ++ | +++ | +++ |
| 2/8 | +++ | +++ | +++ |
| 2/9 | +++ | +++ | +++ |
| 2/10 | +++ | +++ | +++ |

The IC₅₀ ranges for the cytokines are determined with the cytokine assay as described herein: +++: <25 nM; ++: 25 nM to <100 nM.

### Example 201: Oncolead Profiling in a Panel of 5 Prostate Cancer Cell Lines

Compounds of Example 1, 1/1 and 2/8 (10-fold serial dilutions: 30 PM, 3 PM, 300 nM, 30 nM, 3 nM, 0.3 nM) were tested at Oncolead (www.oncolead.com) in prostate cancer cell line 22RV1, DU145, LNCAP, PC3 and VCaP (72 and 120 h treatment) using paclitaxel (30 μM, 3 μM, 300 nM, 30 nM, 3 nM, 0.3 nM) and enzalutamide (60 μM, 6 μM, 600 nM, 60 nM, 6 nM, 0.6 nM) as internal references. Cell growth was assessed by measuring protein content using sulforhodamin B. Growth inhibition was calculated by (T_{CMD}−T₀)/_{TNCT72h}−T₀)*100% with T_{CMD} is the tumor growth at 72 h with the respective compound concentration. To is the protein content of the cell line before treatment and T_{NCT72h} is the tumor growth of the DMSO control.

Compounds of Example 1, 1/1 and 2/8 show a strong growth inhibition in 4 of 5 prostate cancer cell lines. No effect was seen with the highest dose (30 μM) for VCaP. For the other 4 cell lines, most compounds showed an 50% growth inhibition (GI₅₀) in the lower nM range. In addition, Compounds of Example 1, 1/1 and 2/8 were able to reduce growth inhibition for an average of 80% (VCaP cell line not included) with the highest concertation (30 μM). Compounds of Example 1, 1/1 and 2/8 have a higher activity in growth inhibition as reference compound enzalutamide (currently available treatment for CRPC). In addition, compounds Example 1, 1/1 and 2/8 seem to be in a similar range as reference compound paclitaxel.

| Example # | 22RV1 | PC3 | DU145 | VCAP | LNCAP |
|---|---|---|---|---|---|
| 1 | +++ | +++ | +++ | 0 | ++ |
| 1/1 | +++ | +++ | +++ | 0 | +++ |
| 2/8 | +++ | +++ | +++ | 0 | + |
| enzalutamide | 0 | 0 | 0 | + | + |
| paclitaxel | +++ | +++ | +++ | +++ | +++ |

The GI₅₀ ranges for the tumor cell lines are determined with the sulforhodamin B assay as described herein: +++: <25 nM; ++: 25 nM to <100 nM; +: 100 nM to <5 μM; 0: ≥5 μM.

### Example 202: Inhibition of Viability in 24 Different Hematological Cell Lines

The compound of Example 1 was tested at Oncotest GmbH (www.oncotest.com) according their standard procedure in the following cell line types: Acute lymphoblastic leukemia (ALL): CCRFCEM, CCRFCEM/VCR, MOLT4 and JURKAT Acute myelogenous leukemia (AML): HL60, KG1, NOMO1, OCIAML2, PL21 and MV411 Chronic myelogenous leukemia (CML): EM2, JURLMK1, K562, KCL22 and MEGO1 Non-Hodgkin lymphoma (NHL): RAJI, HUT78, MYLA and U937 Multiple myeloma (MM): RPMI8226, L363, IM9, LP1 and NCIH929 Compound Example 1 showed a potent inhibition of viability in 24 different hematological cell lines with a geometrical mean IC₅₀ of below 25 nM:

| Cell line | Name | IC₅₀ range |
|---|---|---|
| ALL | CCRFCEM | ++ |
| ALL | CCRFCEM/VCR | ++ |
| ALL | MOLT4 | +++ |
| ALL | JURKAT | +++ |
| AML | HL60 | +++ |
| AML | KG1 | +++ |
| AML | NOMO1 | + |
| AML | OCIAML2 | ++ |
| AML | PL21 | + |
| AML | MV411 | +++ |
| CML | EM2 | ++ |
| CML | JURLMK1 | +++ |
| CML | K562 | 0 |
| CML | KCL22 | ++ |
| CML | MEG01 | 0 |
| NHL | RAJI | +++ |
| NHL | HUT78 | +++ |
| NHL | MYLA | + |
| NHL | U937 | ++ |
| MM | RPMI8226 | +++ |
| MM | L363 | +++ |
| MM | IM9 | +++ |
| MM | LP1 | ++ |
| MM | NCIH929 | +++ |

IC₅₀ ranges for the tumor cell lines as described herein: +++: <10 nM; ++: 10 nM to <100 nM; +: 100 nM to <1 μM; 0: ≥1 μM.

### Example 203: Viability and Differentiation in Different Solid Cancer Cell Lines

Compound Example 1 and Example 1/1 demonstrate selectivity towards some cancer cell lines (e.g. HEK293 (kidney), A2780 (ovary), MT3 and MCF7 (breast), NCIH460 (lung), A-375 (skin), HCT15 (colon), HT1080 (connective tissue) with IC₅₀'s ranging from 1 to 25 nM.

### Example 204: Oncolead Profiling in a Panel of More than 75 Cancer Cell Lines

The compound of Example 1 and Example 1/1 was tested in vitro in more than 75 representative cancer cell lines at Oncolead (www.oncolead.com) according their standard procedure. The obtained data is summarised in FIG. 1 (Example 1) and FIG. 2 (Example 1/1). A negative Z-ccore indicates, that the compound exhibits an in vitro anticancer activity towards the given cancer cell line. Example 1 and Example 1/1 exhibits an in vitro anticancer activity towards a broad spectrum of therapeutic cancer indication.

### Example 205: Oncotest Profiling in 3 Different Cell Lines for Acute Myelogenous Leukemia

Aim of this study was to evaluate compounds of Example 1, 1/1, 2/2, 2/5 and 2/6 in a proliferation assays. The 5 test compounds were tested in a CellTiter-Blue® based proliferation assay in three AML cell lines (HL-60, THP-1 and MOLM-13). The compounds were tested at 10 concentrations in half-log increments (highest concentration 30 μM or 100 μM) in duplicates. The incubation period with the applied compounds was fixed to 72 hours.

| | IC₅₀ range for | IC₅₀ range for | IC₅₀ range for |
|---|---|---|---|
| Example # | cell line HL-60 | cell line MOLM-13 | cell line THP-1 |
| 1 | +++ | +++ | ++ |
| 1/1 | +++ | +++ | +++ |
| 2/2 | +++ | +++ | ++ |
| 2/5 | +++ | +++ | +++ |
| 2/6 | +++ | +++ | ++ |

Geometric mean relative IC₅₀ ranges for the tumor cell lines as described herein: +++: <10 nM; ++: 10 nM to <100 nM.

### Example 206: Androgen Receptor RNA Level Expression

The prostate cancer cell line 22Rv1 was treated for 48 h with 1 μM Example compound 1, 1/1, 1/2, 2/1, 2/2, 2/3, 2/4, 2/5, 2/6, 2/7, 2/8, 2/9 and 2/10 and the reference compounds SR2211 and XY018 (Nat. Med. 2016; 22:488). RNA was extracted using the RNA isolation kit XS (Macherey-Nagel) and following the manufacture's manual. RNA quantity was measured with the Nanodrop before reverse transcription using Superscript II (Invitrogen #100004925). For the quantitative real-time PCR with SYBR-Green (Qiagen #204074), the following primers were used: AR forward-ACA TCA AGG AAC TCG ATC GTA TCA TTG C and reverse-TTG GGC ACT TGC ACA GAG AT, AR-V7: forward-CCA TCT TGT CGT CTT CGG AAA TGT TAT GAA GC and reverse-TTT GAA TGA GGC AAG TCA GCC TTT CTT. The qRT-PCR proceeded as follows: denaturation for 5 min 95° C.; 40 cycles of “20 sec at 95° C. and 15 sec at 60° C.” and at the end of the last cycle, the melting temperature of the products was determined by gradually increasing it from 60° C. to 99° C. CT values from all samples were normalized to two different reference (housekeeping) genes using the mean CT value of the GAPDH and YWHAZ (reference genes). Next the values are normalized to the untreated control (vehicle/DMSO).

Literature shows that prostate cancer initiation and disease progression are driven by androgen receptor (AR) signaling. Moreover, malignant prostate cancer cell lines express RORγ, which drives androgen receptor (AR) overexpression (Nat. Med. 2016; 22:488). Next to overexpression of full-length AR (AR-FL), expression of AR variants as AR-V7, are contributing to the resistance of therapies that target the AR-axis. Example compound 1, 1/1, 1/2, 2/1, 2/2, 2/3, 2/4, 2/5, 2/6, 2/7, 2/8, 2/9 and 2/10 show a strong reduction in full length AR (AR-FL) as well as AR-V7 mRNA expression. Literature has shown that both reference compounds do reduce AR-FL and AR-V7 at 5 μM in the VCaP cell line. Since we do not see an effect in our assay with 1 μM, this indicates that the examples of the present invention are more active compared to the reference compounds in the 22Rv1 cell line.

| Example # | AR-FL | AR-V7 |
|---|---|---|
| 1 | 0.4424 | 0.3705 |
| 1/1 | 0.4357 | 0.3952 |
| 1/2 | 0.5245 | 0.5094 |
| 2/1 | 0.4244 | 0.2278 |
| 2/2 | 0.3796 | 0.1985 |
| 2/3 | 0.4340 | 0.2547 |
| 2/4 | 0.4345 | 0.2542 |
| 2/5 | 0.4540 | 0.2390 |
| 2/6 | 0.5494 | 0.3045 |
| 2/7 | 0.4250 | 0.2276 |
| 2/8 | 0.4489 | 0.3482 |
| 2/9 | 0.4993 | 0.2928 |
| 2/10 | 0.5315 | 0.2267 |
| SR2211 | 1.058 | 1.059 |
| XY018 | 1.111 | 1.237 |

### Example 207: Androgen Receptor Protein Expression

22Rv1 cells were treated with 6 μM of Example 1, 1/1 and 2/8 or 6 μM references SR2211, XY018, MDV3100 (enzalutamide) or DMSO control. After 48 h, cell were lysed using cell extraction buffer (Life Technologies #FNN0011) for western blot. Samples were loaded on a NuPage 4-12% Bis-Tris gel and transferred to a nitrocellulose membrane. The membrane is then incubation for 1 hour in blocking buffer (5% milk in 20 mM Tris, 150 mM NaCl, pH 7.5, 0.05% Tween 20 [TBS-T]). After this incubation, the primary antibody is added and incubated overnight at 4° C. After washing, the membrane was incubated with the secondary antibody for 1 h. Visualization of the bands was performed using the femto maximum sensitivity substrate reagent and the luminescence signal recorded with the Fusion FX7 (Vilber). For protein quantification, InageJ was used. For analysis, a monoclonal mouse anti-human AR (Santa Cruz Biotechnology, iNc, #SC-7305) and a monoclonal rabbit anti-GAPDH antibody (Cell Signaling Technologies, #2118) were used.

Example 1, 1/1 and 2/8 showed a strong reduction in AR-FL as well as AR-V7 protein levels. Also at protein level, Example 1, 1/1 and 2/8 seem to be more potent in reducing AR-FL and AR-V7 protein levels in 22Rv1.

| Example # | AR-FL | AR-V7 |
|---|---|---|
| 1 | 0.474 | 0.368 |
| 1/1 | 0.482 | 0.323 |
| 2/8 | 0.421 | 0.309 |
| MDV3100 | 0.886 | 0.704 |
| SR2211 | 0.675 | 0.728 |
| XY018 | 0.588 | 0.653 |

### Example 208: Expression of the Androgen Receptor Target Genes

The prostate cancer cell line 22Rv1 was treated for 48h with 1 μM Example compound 1, 1/1, 2/1, 2/2, 2/3, 2/4, 2/5, 2/6, 2/7, 2/8, 2/9 and 2/10 and reference compounds SR2211 and XY018. RNA was extracted using the RNA isolation kit XS (Macherey-Nagel) and following the manufacture's manual. RNA quantity was measured with the Nanodrop before reverse transcription using Superscript II (Invitrogen #100004925). For the quantitative real-time PCR with SYBR-Green (Qiagen #204074), the following primers were used:

KLK2:
forward-
CAA CAT CTG GAG GGG AAA GGG
and
reverse-AGG CCA AGT GAT GCC AGA AC.
KLK3:
forward-
GGA AAT GAC CAG GCC AAG AC
and
reverse-
CCA GCT TCT GCT CAG TGC TT.
NKX3.1:
forward-
CCA TAC CTG TAC TGC GTG GG
and
reverse-
TGC ACT GGG GGA ATG ACT TA.
FKBP5:
forward-
GGG AAG ATA GTG TCC TGG TTA G
and
reverse-
GCA GTC TTG CAG CCT TAT TC.
CAMKK2:
forward-
TGA AGA CCA GGC CCG TTT CTA CTT
and
reverse-
TGG AAG GTT TGA TGT CAC GGT GGA.
ATAD2:
forward-
CAC CGA GTA CTC CTG TGG CTTG
and
reverse-
TCT AGC TCG AGT CAT TCG CAG AAC AC.

qRT-PCR was performed using SYBR-Green (Qiagen #204074) reagents in a PCR cycler (Gorbett Research) Rotor Gene RG3000 with the following program: denaturation for 5′ at 95° C.; 40 cycles of 20″ at 95° C. and 15″ at 60° C.″ and at the end of the last cycle, the melting temperature of the products was determined by gradually increasing from 60° C. to 99° C. CT values from all samples were normalized to reference (housekeeping) genes using the mean CT value of the GAPDH and YWHAZ (reference genes). Next the values are put relative to the untreated control (vehicle/DMSO).

Wang et al. described KLK2, KLK3, NKX3.1, FKBP5, CAMKK2 and ATAD2/ANCCA as AR-target genes. Compounds Example 2/2, 2/3, 2/4, 2/5, 2/6, 2/7, 2/8 and 2/10 show a strong inhibition on KLK2 mRNA level expression, the other Example compounds show a moderate effect and the reference compounds do show none to a slight reduction in KLK2 with this concentration and in this cell line. This shows that compounds of the present invention are more potent. In addition, all Example compounds tested show a strong inhibition of the AR-target gene expression NKX3.1 and FKBP5.

| Example # | KLK2 | KLK3 | NKX3.1 | FKBP5 | CAMKK2 | ATAD2/ANCCA |
|---|---|---|---|---|---|---|
| 1 | 0.598 | — | — | — | 0.884 | — |
| 1/1 | 0.742 | — | — | — | 0.883 | — |
| 2/1 | 0.569 | — | 0.399 | 0.344 | — | — |
| 2/2 | 0.341 | — | 0.255 | 0.246 | — | — |
| 2/3 | 0.411 | — | 0.357 | 0.245 | — | — |
| 2/4 | 0.426 | — | 0.359 | 0.298 | — | — |
| 2/5 | 0.377 | — | 0.307 | 0.271 | — | — |
| 2/6 | 0.428 | — | 0.440 | 0.338 | — | — |
| 2/7 | 0.436 | — | 0.305 | 0.275 | — | — |
| 2/8 | 0.608 | 1.152 | 0.293 | 0.381 | 1.149 | 0.817 |
| 2/9 | 0.412 | — | 0.482 | 0.315 | — | — |
| 2/10 | 0.379 | — | 0.322 | 0.277 | — | — |
| SR2211 | 1.284 | — | — | — | 1.009 | — |
| XY018 | 0.831 | — | — | — | 0.959 | — |

Data are shown relative to vehicle control (DMSO).

### Example 209: RORγt Inhibition

The in vitro inhibition of RORγt was measured using Indigo Biosciences Human RAR-related Orphan Receptor, Gamma Reporter Assay System (Product #11304001-32) as described in the technical manual. In brief, 200 μL of Reporter Cells is dispensed into wells of the assay plate and preincubated for 4-6 h. Following the pre-incubation period, culture media are discarded and 200 μL/well of the prepared 1×-concentration treatment media are added. Following 22-24 h incubation, treatment media are discarded and Luciferase Detection Reagent is added. The intensity of light emission (in units of ‘Relative Light Units’; RLU) from each assay well is quantified using a plate-reading luminometer. Average values of RLU were determined for each treatment concentration. Non-linear regression analyses were performed and IC₅₀ values determined using GraphPad Prism software. Provided inverse-agonists ursolic acid served as positive control. The following results were obtained:

| Example # | IC₅₀ range |
|---|---|
| 1 | +++ |
| 1/1 | +++ |
| 1/2 | ++ |
| 2 | +++ |
| 2/7 | + |
| 2/8 | ++ |

IC₅₀ ranges for the RORγt assay as described herein: +++: <10 nM; ++: 10 nM to <100 nM; +: 100 nM to <1 μM.

### Example 210: Clinical Trial Assessing Activity of a Compound According to Formula (I) in Prostate Cancer Patients

In a first step of a clinical trial, the safety, tolerability and maximum tolerated dose (MTD) has to be evaluated. Metastatic prostate cancer patients in progression of their prostate cancer are enrolled in several success cohorts of increasing dose of a compound according to Formula (I). A predefined number of patients per cohort (e.g. 6 patients) will be enrolled. After an initial period (e.g. first 28 days of treatment with a compound according to Formula (I)) for all patients is completed, the pre-defined criteria for dose-limiting toxicity (DLT) will be assessed. If none of the patients experienced DLT, an independent safety committee will then vote to advance to the next higher dose level. If only few patients (e.g. 1 or 2) experienced DLT, then additional patients may be enrolled at the current dose level. If more patients (e.g. 3 or more) reached DLT, then the MTD was established at the next lower dose. Until MTD has been established, the dose levels are further increased and a new patients cohort will be started. Patient participating in this dose escalation portion of the trial are usually treated as monotherapy (no other prostate cancer therapies allowed) and can continue treatment of the compound according to Formula (I) until disease progression is established. For this reason, repeated assessments of prostate cancer progression marker are performed, including biochemical (e.g. PSA), circulating tumor cell assessment and medical imaging. Patients showing disease progression will terminate treatment.

In a second part of the trial, an expanded treatment cohort will then be dosed using the MTD of a compound according to Formula (I). Focus of this trial part is the preliminary assessment of antitumor activity of the compound according to Formula (I). For this second part, non-metastatic or metastatic prostate cancer patients will received either monotherapy or will be allowed to also stay on their previous antiandrogenic therapy as combination therapy. The latter would allow to also assess whether a compound according to Formula (I) re-sensitize the prostate cancer to antiandrogenic therapy as one of the consequence of using a compound according to Formula (I) in addition to assessing direct anti-tumor activity. Generally, this expansion treatment cohort will consist of approximately 20-30 patients to allow the activity assessment in a large enough patient population. Despite the progression markers mentioned above, many more biomarkers, biopsies, immunohistochemistry or other assessments are made to substantiate and characterize the antitumor activity of a compound according to Formula (I).

Based on the results of the expanded treatment cohort, regulatory Phase 2/3 trials are then initiated. The most applicable patient population will depend on the type of antitumor activity found in these earlier trials. The primary endpoint in such regulatory trials are often progression- or metastasis-free survival.

### FIGURE CAPTIONS

FIG. 1: Z-score for Example 1 in a panel of more than 75 cancer cell lines (from Oncolead).

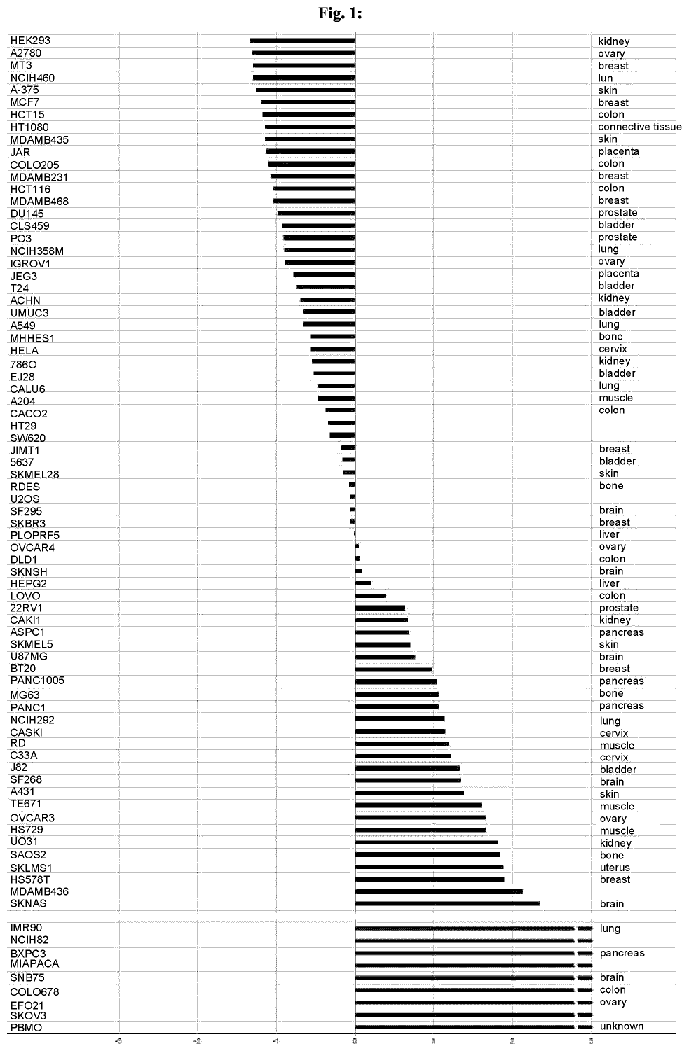

FIG. 2: Z-score for Example 1/1 in a panel of more than 75 cancer cell lines (from Oncolead).

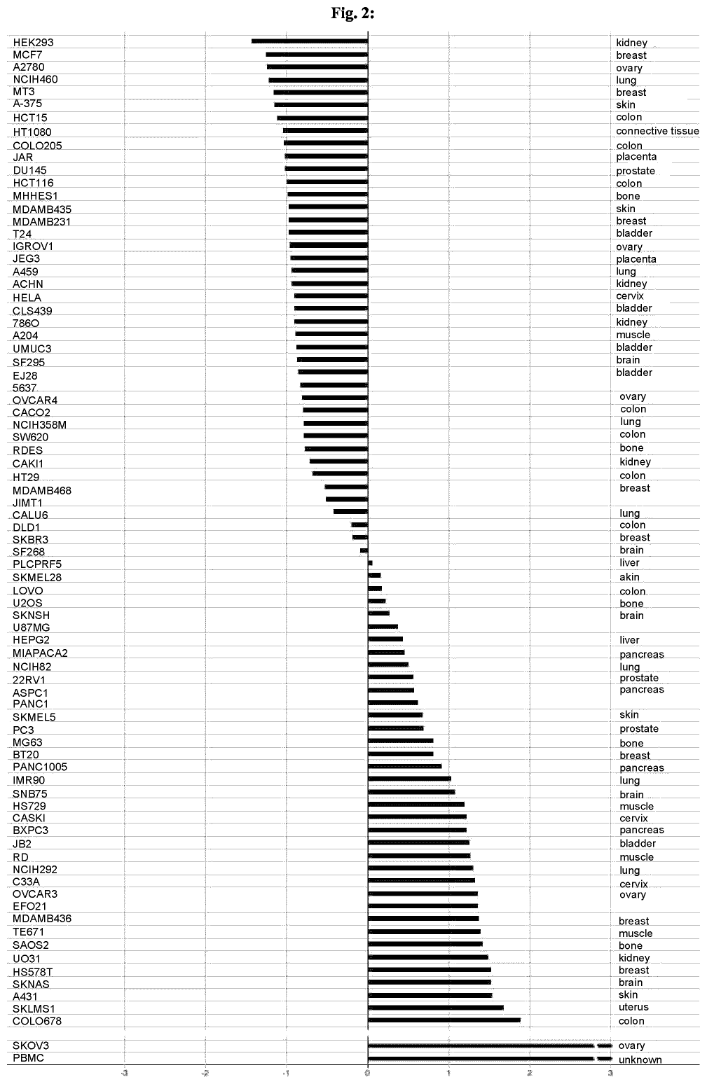

1. A method of treating a cancer in a subject in need thereof, comprising administering to the subject a compound according to Formula (I): 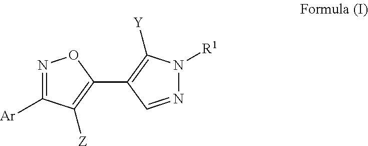
or a pharmaceutically acceptable salt, a solvate, a solvate of a salt, a hydrate or a polymorph thereof wherein
Ar is selected from the group consisting of phenyl and heteroaryl, each of which is optionally substituted by one or more independently selected substituents R^{Ar};
R^{Ar} is selected from the group consisting of halogen, —OH, —CN, alkoxy, haloalkoxy, alkyl, haloalkyl, mono- or dialkylamino-alkyl, mono- or di-alkylamino-alkoxy, —COOR′, —CONHR′, —CO—R′, —SO₂NHR′, —NH—CO—R′, —NO₂, —NH—SO₂—R′, —SO₂—R′, benzyloxy, —CO— heterocyclyl, —CO-cycloalkyl, —CONH-cycloalkyl, —CONH-heterocyclyl, —O-alkyl-heterocyclyl, —O-alkyl-cycloalkyl, (2-oxa-6-azaspiro[3.3]hept-6-yl)—C₁₋₄-alkoxy, amino, arylalkyl, cycloalkyl, heterocyclyl, phenyl and heteroaryl, wherein each of said alkoxy, arylalkyl, alkyl, cycloalkyl, heterocyclyl, phenyl and heteroaryl groups is optionally substituted by one or more substituents independently selected from alkyl, haloalkyl, halogen and OH;
R′ is independently selected from the group consisting of H, OH, alkyl and haloalkyl;
Z is selected from the group consisting of H, halogen, —CO—R^{Z}, —CH₂—O—R^{Z}, —CO—CH₂—R^{Z}, —CO—CH₂—O—R^{Z}, —COOR^{Z}, —NHCO—R^{Z}, —CO—NHR^{Z}, —N(R^{Z})₂, —CN, —NHCOOR^{Z}, —SO₂—R^{Z}, —SO₂NHR^{Z}, -alkyl-O—R^{Z}, -alkyl-O-alkyl-O—R^{Z}, amino, alkyl, phenyl, heteroaryl, heterocyclyl and cycloalkyl, wherein each of said alkyl, phenyl, heteroaryl, heterocyclyl and cycloalkyl groups is optionally substituted by one or more substituents independently selected from the group consisting of halogen, alkyl, alkoxy, haloalkyl, —COO-alkyl, OH and cycloalkyl;
R^{Z} is selected from the group consisting of H, halogen, —OH, alkyl, haloalkyl, cycloalkyl, heterocyclyl, phenyl and heteroaryl;
Y is selected from H, halogen, haloalkyl, alkyl or an alkylester;
R¹ is selected from aryl, heteroaryl, heterocyclyl or alkyl, which can be substituted by one or more substituents R″; wherein
R″ is independently selected from H, —CO₂R′″, —CONHR′″, —CR′″O, —SO₂N(R′″)₂,—SO₂NHR′″, —NR′″—CO-haloalkyl, —NO₂, —NR′″—SO₂-haloalkyl, —NR′″—SO₂-alkyl, —SO₂-alkyl, —NR′″—CO-alkyl, —CN, alkyl, cycloalkyl, aminoalkyl, alkylamino, alkoxy, —OH, —SH, alkylthio, hydroxyalkyl, hydroxyalkylamino, halogen, haloalkyl, haloalkoxy, amino, heterocyclyl, aryl, haloaryl, haloarylalkyl, arylalkyl or heteroaryl;
R′″ independently represents H, haloalkyl, hydroxyalkyl, amino, alkoxy, —N═C(R″)₂, —NR″—CO—R″, —CR″O, —CO₂R″, alkyl, cycloalkyl, aryl, haloaryl, haloarylalkyl, heteroaryl, heterocyclyl, arylalkyl or aminoalkyl, which are optionally substituted by one or more substituents R″;
wherein the longest chain allowed in R¹ are three coupled substituents R″ and/or R′″,
or, alternatively R¹ is a group of the structure 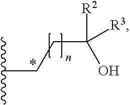
wherein
n is 0 or 1;
R² is H, deuterium or methyl;
R³ is methyl, trifluoromethyl, ethyl, or taken with R² together forms a cyclopropyl group;
or
n is 1, R² is H, deuterium or methyl and R³ forms a methylene bridge to the carbon atom marked *.

2. The method according to claim 1, wherein
Ar is phenyl, which is optionally substituted by one or more independently selected from fluoro, chloro, alkoxy, fluoroalkoxy, —CO-heterocyclyl and —O-alkyl-heterocyclyl.
Z is selected from the group consisting of —CO—R^{Z}, —CH₂—O—R^{Z} and heteroaryl, wherein said heteroaryl is optionally substituted by one or more substituents independently selected from the group consisting of fluoro, chloro, alkyl, alkoxy, fluoroalkyl and OH;
R^{Z} is selected from the group consisting of alkyl, fluoroalkyl, cycloalkyl and heterocyclyl;
Y is selected from alkyl or fluoroalkyl;
R¹ is selected from aryl, which can be substituted by one or more substituents selected from chloro, fluoro, —CN, alkyl, fluoroalkyl, alkoxy or fluoroalkoxy;
or a group of the structure 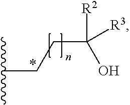
wherein
n is 0 or 1;
R² is H, deuterium or methyl;
R³ is methyl, trifluoromethyl, ethyl, or taken with R² together forms a cyclopropyl group;
or
n is 1, R² is H, deuterium or methyl and R³ forms a methylene bridge to the carbon atom marked *.

3. The method according to claim 1, wherein
Ar is selected from 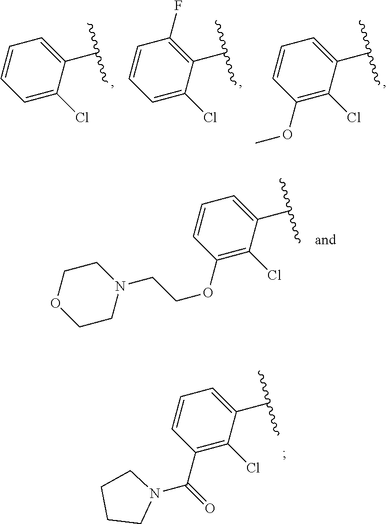
Z is selected from 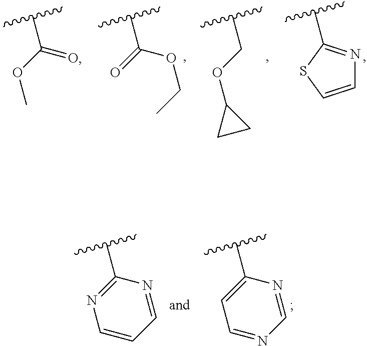
Y is CF₃;
R¹ is selected from

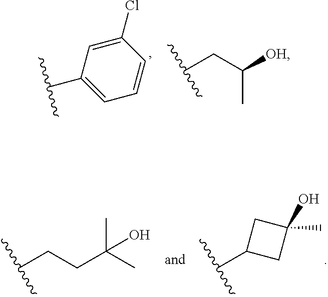

4. The method according to claim 1, wherein the compound according to Formula (I) is selected from

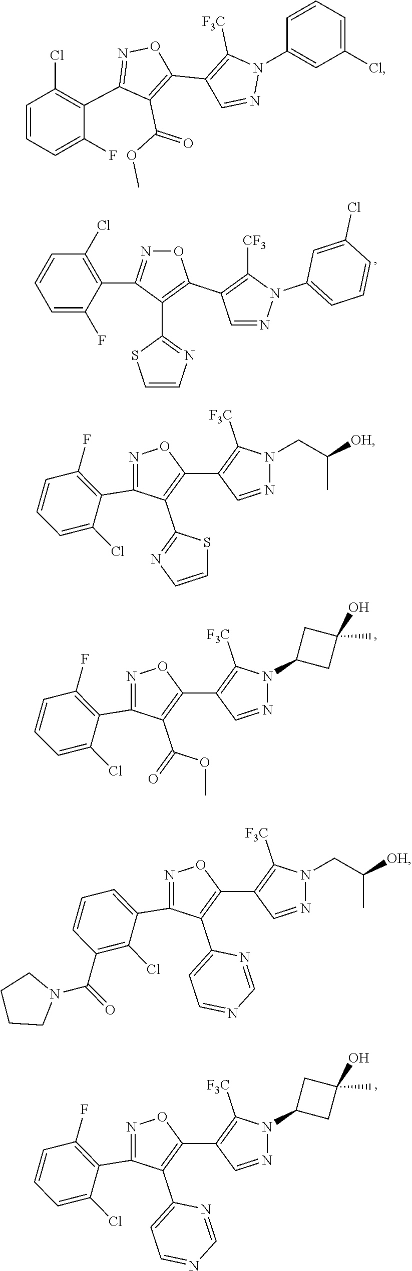 [SMILES: COC(=O)c1c(-c2c(F)cccc2Cl)noc1-c1cnn(-c2cccc(Cl)c2)c1C(F)(F)F ; Fc1cccc(Cl)c1-c1noc(-c2cnn(-c3cccc(Cl)c3)c2C(F)(F)F)c1-c1nccs1 ; C[C@H](O)Cn1ncc(-c2onc(-c3c(F)cccc3Cl)c2-c2nccs2)c1C(F)(F)F ; COC(=O)c1c(-c2c(F)cccc2Cl)noc1-c1cnn([C@H]2C[C@@](C)(O)C2)c1C(F)(F)F ; C[C@H](O)Cn1ncc(-c2onc(-c3cccc(C(=O)N4CCCC4)c3Cl)c2-c2ccncn2)c1C(F)(F)F ; C[C@]1(O)C[C@@H](n2ncc(-c3onc(-c4c(F)cccc4Cl)c3-c3ccncn3)c2C(F)(F)F)C1]

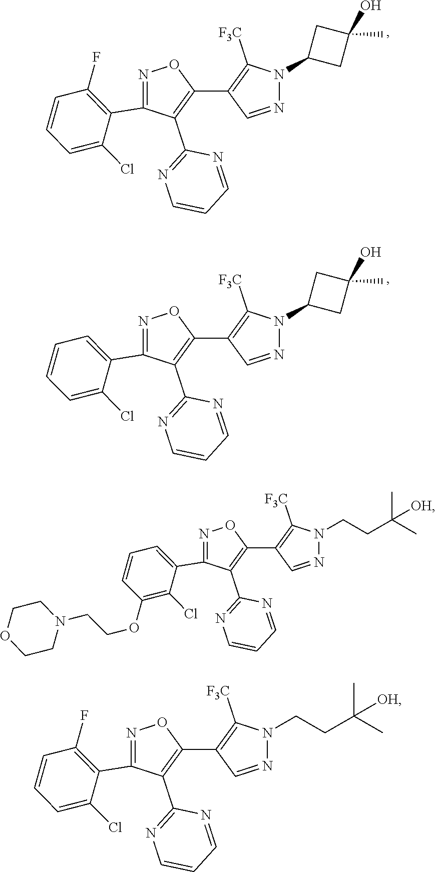

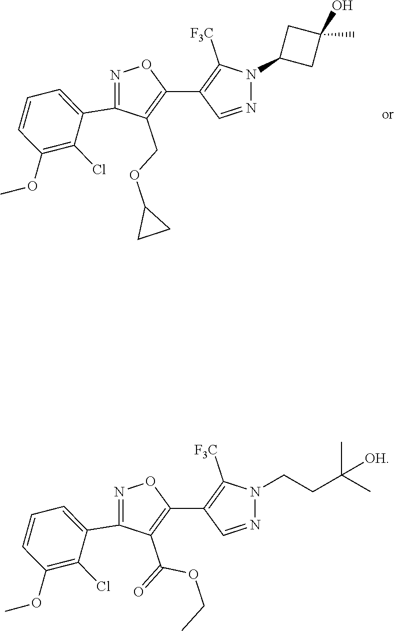

5. The method according to claim 1, wherein the cancer is selected from the group consisting of prostate cancer, breast cancer, lung cancer, ovarian cancer, bladder cancer, endometrial cancer, liver cancer, glioblastoma, B-cell lymphoma and colon cancer.

6. The method according to claim 1, wherein the cancer is resistant to a chemotherapeutic agent selected from tamoxifen, a taxane, an anthracenedione or combinations thereof.

7. The method according to claim 1, wherein the prostate cancer is a castration-resistant prostate cancer.

8. The method according to claim 1, wherein the subject is a human in need of cancer treatment.

9. The method according to claim 1, wherein said compound of Formula (I) or a pharmaceutically acceptable salt, a solvate, a solvate of a salt, a hydrate or a polymorph thereof is provided as a suitable pharmaceutical composition.

10. The method according to claim 1, wherein said compound of Formula (I) or a pharmaceutically acceptable salt, a solvate, a solvate of a salt, a hydrate or a polymorph thereof is administered to said subject in an effective dosage regimen.

11. The method according to claim 10, wherein said method further comprises administering to the subject an effective amount of an anticancer drug.

12. The method according to claim 11, wherein the compound according to Formula (I) enhances the therapeutic effect of the anticancer drug.

13. The method according to claim 12, wherein the compound according to Formula (I) reverses or reduces cancer cell resistance to the anticancer drug and/or sensitizes cancer cells to the anticancer drug.

14. A kit comprising one or more compounds according to Formula (I) or a pharmaceutically acceptable salt, a solvate, a solvate of a salt, a hydrate or a polymorph thereof as defined in claim 1 and a label describing a method of administering one or more compounds according to Formula (I) or a pharmaceutically acceptable salt, a solvate, a solvate of a salt, a hydrate or a polymorph thereof and/or one or more anticancer drugs.

15. The method according to claim 9, wherein the suitable pharmaceutical composition is selected from the group consisting of a tablet, capsule, granule, powder, sachet, reconstitutable powder, dry powder inhaler and chewable.

16. The method according to claim 10, wherein the effective dosage regimen is from one to three times a day with a cumulative daily dose of 150 to 900 mg.


SEQUENCE LISTING (16 sequences)

SEQ ID NO: 1 - DNA, 28 bp, synthetic construct
  misc_feature 1..28: note = PCR Primer
  source 1..28: mol_type = other DNA
acatcaaggaactcgatcgtatcattgc

SEQ ID NO: 2 - DNA, 20 bp, synthetic construct
  misc_feature 1..20: note = PCR Primer
  source 1..20: mol_type = other DNA
ttgggcacttgcacagagat

SEQ ID NO: 3 - DNA, 32 bp, synthetic construct
  misc_feature 1..32: note = PCR Primer
  source 1..32: mol_type = other DNA
ccatcttgtcgtcttcggaaatgttatgaagc

SEQ ID NO: 4 - DNA, 27 bp, synthetic construct
  misc_feature 1..27: note = PCR Primer
  source 1..27: mol_type = other DNA
tttgaatgaggcaagtcagcctttctt

SEQ ID NO: 5 - DNA, 21 bp, synthetic construct
  misc_feature 1..21: note = PCR Primer
  source 1..21: mol_type = other DNA
caacatctggaggggaaaggg

SEQ ID NO: 6 - DNA, 20 bp, synthetic construct
  misc_feature 1..20: note = PCR Primer
  source 1..20: mol_type = other DNA
aggccaagtgatgccagaac

SEQ ID NO: 7 - DNA, 20 bp, synthetic construct
  misc_feature 1..20: note = PCR Primer
  source 1..20: mol_type = other DNA
ggaaatgaccaggccaagac

SEQ ID NO: 8 - DNA, 20 bp, synthetic construct
  misc_feature 1..20: note = PCR Primer
  source 1..20: mol_type = other DNA
ccagcttctgctcagtgctt

SEQ ID NO: 9 - DNA, 20 bp, synthetic construct
  misc_feature 1..20: note = PCR Primer
  source 1..20: mol_type = other DNA
ccatacctgtactgcgtggg

SEQ ID NO: 10 - DNA, 20 bp, synthetic construct
  misc_feature 1..20: note = PCR Primer
  source 1..20: mol_type = other DNA
tgcactgggggaatgactta

SEQ ID NO: 11 - DNA, 22 bp, synthetic construct
  misc_feature 1..22: note = PCR Primer
  source 1..22: mol_type = other DNA
gggaagatagtgtcctggttag

SEQ ID NO: 12 - DNA, 20 bp, synthetic construct
  misc_feature 1..20: note = PCR Primer
  source 1..20: mol_type = other DNA
gcagtcttgcagccttattc

SEQ ID NO: 13 - DNA, 24 bp, synthetic construct
  misc_feature 1..24: note = PCR Primer
  source 1..24: mol_type = other DNA
tgaagaccaggcccgtttctactt

SEQ ID NO: 14 - DNA, 24 bp, synthetic construct
  misc_feature 1..24: note = PCR Primer
  source 1..24: mol_type = other DNA
tggaaggtttgatgtcacggtgga

SEQ ID NO: 15 - DNA, 22 bp, synthetic construct
  misc_feature 1..22: note = PCR Primer
  source 1..22: mol_type = other DNA
caccgagtactcctgtggcttg

SEQ ID NO: 16 - DNA, 26 bp, synthetic construct
  misc_feature 1..26: note = PCR Primer
  source 1..26: mol_type = other DNA
tctagctcgagtcattcgcagaacac